(19) United States
(12) Patent Application Publication
(10) Pub. No.: US 2022/0233535 A1
(43) Pub. Date: Jul. 28, 2022

(54) USE OF INHIBITORS OF YAP/TAZ FOR THE TREATMENT OF CANCER

(71) Applicant: Georgetown University, Washington, DC (US)

(72) Inventors: Chunling Yi, Washington, DC (US); Shannon M. White, Arlington, VA (US); Jeffrey Field, Philadelphia, PA (US)

(21) Appl. No.: 17/608,981

(22) PCT Filed: Apr. 26, 2020
(86) PCT No.: PCT/US20/30001

§ 371 Date: Nov. 4, 2021

Related U.S. Application Data
(60) Provisional application No. 62/843,559, filed on May 5, 2019.
(60) Provisional application No. 62/844,117, filed on May 6, 2019.

(51) Int. Cl. A61K 31/506; A61K 31/409; A61K 31/4725; A61K 31/137; A61K 31/427; A61K 31/407; A61K 31/4745; A61K 38/48; A61K 31/519; A61K 31/4523; A61K 31/4184; A61K 31/18; A61P 35/00

(52) U.S. Cl. CPC A61K 31/506; A61K 31/409; A61K 31/4725; A61K 31/137; A61K 31/427; A61K 31/407; A61P 35/00; A61K 38/4893; A61K 31/519; A61K 31/4523; A61K 31/4184; A61K 31/18; A61K 31/4745

(57) ABSTRACT

Methods of treating or preventing cancer, or treating or preventing noncancerous tumors or lesions, in a subject in need thereof. The methods involve administering a therapeutically effective amount of one or more inhibitors of the YAP/TAZ pathway to the subject. In addition, methods of inhibiting or preventing glycolysis in cancer cells in a subject, promoting mitochondrial respiration in cancer cells in a subject, and promoting oxidative stress in cancer cells in a subject, by administering a therapeutically effective amount of one or more inhibitors of the YAP/TAZ pathway to the subject.

### CROSS-REFERENCE TO RELATED APPLICATIONS

This application claims benefit of provisional application No. 62/843,559 filed May 5, 2019 and of provisional application No. 62/844,117 filed May 6, 2019, the entireties of which are all herein incorporated by reference.

### STATEMENT REGARDING FEDERALLY SPONSORED RESEARCH OR DEVELOPMENT

This invention was made with government support under grant number P50-CA101942-10 awarded by Dana-Farber/Harvard Cancer Center Kidney Cancer Specialized Program on Research Excellence (SPORE). The government has certain rights in the invention.

### FIELD OF INVENTION

The present invention generally relates to treatment, diagnostic, and compound screening methods involving inhibitors of yes-associated protein 1 (YAP) and transcriptional coactivator with PDZ-binding motif (TAZ).

### BACKGROUND OF THE INVENTION

The Neurofibromatosis Type 2 (NF2) gene encodes the Moesin-ezrin-radaxin-like protein (Merlin) and is a tumor suppressor [1]. Deletions or loss-of-function mutations of NF2 underlie neurofibromatosis type 2 (NF2), which is an inherited syndrome characterized by the development of bilateral vestibular schwannomas, schwannomas from cranial or peripheral nerves, meningiomas, and/or ependymomas [1]. Beyond NF2, somatic NF2 mutations are frequently detected in sporadic schwannomas, meningiomas, ependymomas and mesotheliomas, as well as in thyroid cancer, colorectal cancer, melanoma, renal cell carcinomas (RCCs), and other solid tumors [1].

Merlin/NF2 is primarily localized to the plasma membrane where it has been shown to mediate contact-dependent inhibition of proliferation in normal cells [1]. Loss of Merlin/NF2 triggers deregulation of numerous signaling pathways, including MST1/2-LATS1/2 (Hippo), RAC-PAK, RAS-RAF-MEK-ERK, PI3K-AKT-mTOR, FAK-SRC, STAT3, and a number of receptor tyrosine kinases (RTKs) [2-10]. Despite their prevalent activation in NF2-mutant tumors, clinical trials with drugs targeting mTOR, MEK, and several RTKs have yielded largely disappointing results in treating NF2 [11, 12], underscoring the need to fully explore the molecular mechanisms that govern the growth and survival of NF2-deficient tumors.

One of the best characterized Merlin/NF2-regulated pathways is the Hippo pathway, which regulates tissue homeostasis [13, 14]. As a part of a scaffolding complex also composed of WW45/SAV1 and KIBRA, Merlin/NF2 facilitates the recruitment of MST1/2 and LATS1/2 kinases to the plasma membrane, where MST1/2 phosphorylate and activate LATS1/2 [15-20]. Activated LATS1/2 kinases in turn phosphorylate two paralogous transcriptional co-activators yes-associated protein 1 (YAP) and transcriptional coactivator with PDZ-binding motif (TAZ), resulting in their cytoplasmic sequestration and/or proteasomal degradation [14, 21, 22]. In addition, Merlin/NF2 has been shown to inhibit LATS1/2 ubiquitination and degradation by the CRL4^{DCAF1}E3 ubiquitin ligase within the nucleus [23, 24]. In NF2-mutant tumors, because of the inactivation of LATS1/2, unphosphorylated YAP and TAZ become stabilized and free to enter the nucleus where they bind to and partner with the TEAD family of transcription factors to regulate gene expression [14, 21, 25].

### SUMMARY OF THE INVENTION

The present invention relates to uses associated with the inhibition of YAP and/or TAZ.

Aspects of the present invention relate to methods of treating or preventing cancer in a subject in need thereof, the methods comprising administering a therapeutically effective amount of one or more inhibitors of the YAP/TAZ pathway to the subject. In some embodiments, the cancer is selected from the group consisting of blood cancer, leukemia, lymphoma, skin cancer, melanoma, breast cancer, ovarian cancer, uterine cancer, prostate cancer, testicular cancer, colorectal cancer, stomach cancer, intestinal cancer, bladder cancer, lung cancer, non-small cell lung cancer, pancreatic cancer, renal cell carcinoma, kidney cancer, liver cancer, hepatocarcinoma, brain cancer, head and neck cancer, retinal cancer, glioma, lipoma, throat cancer, thyroid cancer, neuroblastoma, endometrial cancer, myelomas, mesothelioma, and esophageal cancer.

Aspects of the present invention relate to a methods of treating or preventing noncancerous tumors or lesions in a subject in need thereof, the methods comprising administering a therapeutically effective amount of one or more inhibitors of the YAP/TAZ pathway to the subject. In some embodiments, the noncancerous tumors or lesions are associated with neurofibromatosis type 2 (NF2).

Aspects of the present invention relate to methods of inhibiting or preventing glycolysis in cancer cells in a subject in need thereof, the methods comprising administering a therapeutically effective amount of one or more inhibitors of the YAP/TAZ pathway to the subject.

Also, aspects of the present invention relate to methods of promoting or inducing mitochondrial respiration in cancer cells in a subject in need thereof, the methods comprising administering a therapeutically effective amount of one or more inhibitors of the YAP/TAZ pathway to the subject.

In addition, aspects of the present invention relate to methods of promoting or inducing oxidative stress in cancer cells in a subject in need thereof, the methods comprising administering a therapeutically effective amount of one or more inhibitors of the YAP/TAZ pathway to the subject.

Further, aspects of the present invention relate to methods of promoting or inducing lysosome-mediated activation of mitogen-activated protein kinase (MAPK) signaling in cancer cells in a subject in need thereof, the methods comprising administering a therapeutically effective amount of one or more inhibitors of the YAP/TAZ pathway to the subject.

In some embodiments, the cancer cells are selected from the group consisting of skin cancer cells, breast cancer cells, ovarian cancer cells, uterine cancer cells, prostate cancer cells, testicular cancer cells, colorectal cancer cells, stomach cancer cells, intestinal cancer cells, bladder cancer cells, lung cancer cells, non-small cell lung cancer cells, pancreatic cancer cells, kidney cancer cells, liver cancer cells, brain cancer cells, head and neck cancer cells, retinal cancer cells, throat cancer cells, thyroid cancer cells, endometrial cancer cells, and esophageal cancer cells.

In embodiments of the invention, the one or more inhibitors may comprise verteporfin, (R)-PFI 2 hydrochloride, CA3, dasatinib, statins, pazopanib, β-adrenergic receptor agonists, dobutamine, latrunculin A, latrunculin B, cytochalasin D, actin inhibitors, drugs that act on the cytoskeleton, blebbistatitin, botulinum toxin C3, RHO kinase-targeting drugs, or a combination thereof.

In some embodiments, the administration of the one or more inhibitors of the YAP/TAZ pathway is preceded by a step of identifying the subject in need thereof.

In embodiments of the invention, the methods further comprise administering one or more inhibitors of MAPK signaling to the subject. In some embodiments, the one or more inhibitors of MAPK signaling comprises one or more inhibitors of rapidly accelerated fibrosarcoma (RAF)—mitogen-activated extracellular signal-regulated kinase (MEK)—extracellular signal-regulated kinases (ERK) pathway (RAF-MEK-ERK pathway). In some embodiments, the one or more inhibitors of MAPK signaling comprises trametinib, cobimetinib, binimetinib, refametinib, selumetinib, or a combination thereof.

### BRIEF DESCRIPTION OF THE DRAWING FIGURES

The present disclosure will be further explained with reference to the attached drawing figures.

FIG. 1 provides results from the Example relating to how YAP/TAZ can maintain redox balance and prevent oxidative-stress-induced cell death by promoting glycolysis while reducing mitochondrial respiratory capacity. FIG. 1A shows oxidative consumption rates (OCR) of Ctrl and shY/T SN12C cells before or after indicated treatments (n=6) (Rot, Rotenone; AMA, Antimycin A; ***p<0.0005; data represent mean±SD). FIG. 1B shows representative flow cytometry profiles of shY/T SN12C cells treated with Dox for indicated time periods followed by staining with CellROX deep red or MitoTracker deep red FM. FIG. 1C shows representative images of mitochondria IF staining (left) and electron microscopy (EM) analysis (right) of GFP-labeled Ctrl and shY/T SN12C cells (asterisks mark the mitochondria; scale bars, 20 μm (left) and 1 μm (right)). FIG. 1D shows quantification of the length of individual mitochondria captured in EM images (***p<0.0005). FIG. 1E shows western blot (WB) analysis of indicated subunits of oxidative phosphorylation (OXPHOS) complexes I-V in Ctrl and shY/T whole-cell extract (WCE) and mitochondria-enriched subcellular fractions; TUBULIN was used as loading control. FIG. 1F shows representative images (left) and quantification (right) of Ctrl and shY/T SN12C cells co-stained with Mitosox red and MitoTracker green (*p<0.05; scale, 25 μm). FIG. 1G shows simplified schematic illustrating TCA cycle and OXPHOS with metabolites of interest highlighted in blue and OXPHOS inhibitors highlighted in red (Gln, glutamine; Pyr, pyruvate; Mal, malate; Succ, succinate; AA5, Atpenin A5). FIG. 1H shows OCR of permeabilized Ctrl and shY/T SN12C cells before or after indicated treatments (n=5) (***p<0.0005). FIG. 1I shows NAD+/NADH, NADP+/NADPH and GSH/GSSG ratios in Ctrl and shY/T SN12C cells as measured by NAD/NADH-Glo, NADP/NADPH-Glo, and GSH/GSSG-Glo assays (n=3) (**p<0.005; ***p<0.0005). FIG. 1J shows luminescence readings (RLU) from ROS-Glo H202 assay of Ctrl and shY/T SN12C cells after being grown for 24 h in medium containing both glucose and glutamine (++) or deprived of either glucose (−Glc) or glutamine (−Gln) (n=4) (***p<0.0005). FIG. 1K shows percent change in fluorescence of Ctrl and shY/T SN12C cells after being grown for 3 days with glutathione (GSH) in medium deprived of either glucose (−Glc) or glutamine (−Gln) (n=3) (ns, not significant; **p<0.005; data represent mean±SEM).

FIG. 2 provides results from the Example relating to the in vitro and in vivo effects of YAP/TAZ depletion on NF2 mutant tumor cells. FIG. 2A shows WB analysis of YAP and TAZ levels in shY/T SN12C cells grown for 4 days in the presence or absence of Dox (ACTIN was used as loading control). FIG. 2B shows correlation between final BLI measurements prior to dissection and volume of resected tumors. FIG. 2C shows luminescence signal of increasing cell numbers of shY/T SN12C cells grown for 3 days with or without Dox. FIG. 2D shows representative IHC images of YAP or TAZ in a matched region of shY/T Escape and Ctrl SN12C tumors (scale bar=1 mm). FIG. 2E shows percent change in fluorescence of Ctrl and shY/T SN12C cells after grown for 3 days in medium with or without FBS (n=3) (***P<0.0005; data represent mean±SD). FIG. 2F shows WB analysis of YAP and TAZ levels in Ctrl and shY/T SC4 cells after 4 days of Dox treatment (ACTIN was used as loading control). FIG. 2G shows percent change in fluorescence of Ctrl and shY/T SC4 cells after grown for 3 days in medium with or without FBS (n=3) (dashed-line indicates no change in fluorescence; ***P<0.0005; data represent mean±SD). FIG. 2H shows percent change in fluorescence of Ctrl and shY/T SN12C cells after grown for 3 days in normoxia (21% O₂) or hypoxia (2% O₂) (n=3) (ns=not significant; data represent mean±SD). FIG. 2I shows WB analysis of HIF1α, YAP and TAZ levels in Ctrl and shY/T SN12C cells after grown for 6 hours in normoxia (21% O₂) or hypoxia (2% O₂) (ACTIN was used as loading control).

FIG. 3 provides results from the Example relating to how YAP/TAZ may be required for the maintenance of NF2-mutant kidney tumors. FIG. 3A shows a schematic of the experimental design; mice bearing orthotopic Ctrl or shY/T SN12C kidney tumors were switched to a Dox-containing diet once their tumor luminescence flux reached ˜108 photons/seconds via bioluminescent imaging (BLI). FIG. 3B shows logₑ-fold change (FC) in BLI signal from the start of Dox treatment (top) and absolute FC in BLI signal over indicated time periods (bottom) of individual Ctrl (n=5) and shY/T (n=6) tumors (note that the growth trajectories of Ctrl tumors remained largely steady, whereas shY/T tumors regressed initially (shY/T Regress), followed by a period of stagnant growth (shY/T Stagnant) but eventually resumed growth despite continued Dox treatment (shY/T Escape)). FIG. 3C shows representative sequential BLI images of two mice bearing Ctrl or shY/T SN12C orthotopic kidney tumors prior to (Pre) or after 2 weeks of Dox treatment (Post) (scale is in photons/second). FIG. 3D shows Kaplan-Meier survival analysis of mice bearing Ctrl (n=5) or shY/T (n=6) SN12C tumors from the start of Dox treatment (**p<0.005). FIG. 3E shows representative images of IHC staining with indicated antibodies in Ctrl and shY/T tumors harvested during tumor regression (R) or escape (E) (scale bar, 100 μm). FIGS. 3F and 3G shows quantification of Ki67 (3F) and pH2AX (3G) IHC staining as depicted in FIG. 3E (ns=not significant; *p<0.05; **p<0.005; ***p<0.0005).

FIG. 4 provides results from the Example relating to how YAP/TAZ can promote glycolysis and reduce glutamine dependence in NF2 mutant cells. FIG. 4A shows percent change in fluorescence of Ctrl and shY/T SN12C cells after grown for 3 days in medium containing both glucose and glutamine (++), or deprived of either glucose (—Glc) or glutamine (—Gln) (n=3) (ns: not significant; *P<0.05; ***P<0.0005; data represent mean±SD). FIG. 4B shows percentage of AnnexinV/Sytox double positive cells of Ctrl and shY/T SN12C cells after grown for 3 days in medium containing both glucose and glutamine (++), or deprived of either glucose (—Glc) or glutamine (—Gln) (n=3) (ns: not significant; **P<0.005; ***P<0.0005; data represent mean±SD). FIG. 4C shows percent change in fluorescence of Ctrl and shY/T SC4 cells after grown for 3 days in medium containing both glucose and glutamine (++), or deprived of either glucose (—Glc) or glutamine (—Gln) (n=3) (ns: not significant; *P<0.05; ***P<0.0005; data represent mean±SD). FIG. 4D shows percent change in fluorescence of shY/T cells stably expressing TAZ (shY/T+TAZ), shY/T, or Ctrl SN12C cells after grown for 3 days in medium containing both glucose and glutamine (++), or deprived of either glucose (—Glc) or glutamine (—Gln) (n=3) (ns: not significant; ***P<0.0005; data represent mean±SD). FIG. 4E shows percent change in fluorescence of SN12C cells with single knockdown of YAP (shY), TAZ (shT), both (shY/T), or Ctrl after grown for 3 days in medium containing both glucose and glutamine (++), or deprived of either glucose (—Glc) or glutamine (—Gln) (n=3) (ns: not significant; *P<0.05; ***P<0.0005; data represent mean±SD). FIG. 4F shows percent change in fluorescence of Ctrl and shY/T SN12C cells after grown for 3 days in media salt base alone or salt base supplemented with either glucose (+Glc) or glutamine (+Gln) (n=3) (ns=not significant; **P<0.005; data represent mean±SD). FIG. 4G shows luminescence readings (RLU) from Glucose Uptake-Glo Assay of Ctrl and shY/T SN12C cells (n=3) (***P<0.0005; data represent mean±SD). FIGS. 4H and 4I shows extracellular acidification rates (ECARs) of shY/T and shY/T+TAZ (4H) or shY/T+YAP (4I) SN12C cells before or after indicated treatments (n=6) (***P<0.0005; data represent mean±SD). FIG. 4J shows log2 FC in the levels of indicated glycolysis and TCA cycle intermediates in shY/T relative to Ctrl SC4 cells as measured by targeted LC-MS/MS analysis (n=6) (ns=not significant; **P<0.005). FIG. 4K shows percent change in fluorescence of Ctrl and shY/T SN12C cells after grown for 3 days in presence or absence of glucose and/or galactose (Gal) as indicated (n=3) (ns=not significant; *P<0.05; ***P<0.0005; data represent mean±SD). FIG. 4L shows relative mRNA levels of GLUT3 in shY/T cells stably expressing GLUT3 (shY/T+GLUT3) and shY/T SN12C cells as measured by qRT-PCR analysis (n=4) (***P<0.0005). FIG. 4M shows WB analysis of pAKT levels in Ctrl and shY/T SN12C cells treated for 30 minutes with RPMI conditioned medium (CM) collected after a 3-day incubation with a cell-free plate (RPMI), Ctrl (Ctrl CM) or shY/T (shY/T CM) SN12C cells (ERK was used as loading control). FIG. 4N shows WB analysis of pAKT and p4EBP1 levels in Ctrl and shY/T SN12C cells at indicated times after addition of EGF (ACTIN was used as loading control). FIG. 4O shows WB analysis of pAKT and pS6 levels in shY/T and shY/T+MyrAKT SN12C cells (ERK was used as loading control). FIG. 4P shows percent growth of Ctrl and shY/T SN12C cells after grown for 3 days in medium without (++) or with either GSH or EGF supplement (n=3) (dashed-line indicates no growth; ns: not significant; **P<0.005; data represent mean±SD).

FIG. 5 provides results from the Example relating to how YAP/TAZ may be required for the maintenance of NF2-mutant kidney tumors. FIGS. 5A and 5B shows percent change in fluorescence of Ctrl and shY/T SN12C cells cultured for 3 days in medium containing indicated concentrations of Glc (5A) or Gln (5B) (n=3) (dashed line indicates no change in fluorescent signals post to prior to treatment; ns, not significant; *p<0.05; **p<0.005; ***p<0.0005). FIG. 5C shows ECAR of Ctrl and shY/T SN12C cells before or after indicated treatments (n=6) (Glc, glucose; Oligo, Oligomycin; 2-DG, 2-Deoxy-D-glucose; ***p<0.0005). FIG. 5D shows relative mRNA levels of GLUT1-3 in Ctrl and shY/T SN12C cells as measured by qRT-PCR analysis (n=4) (ns, not significant; **p<0.005; ***p<0.0005). FIG. 5E shows log2-FC of indicated glycolytic enzymes (red) and growth factors (blue) transcript levels from microarray analysis of Ctrl and shY/T SN12C cells (n=3) (*p<0.05; **p<0.005; ***p<0.0005; data represent mean±SD). FIG. 5F shows percent change in fluorescence of Ctrl, shY/T, and shY/T SN12C cells stably expressing GLUT3 (shY/T+GLUT3) SN12C cells after being grown for 3 days in medium containing both glucose and glutamine (++) or deprived of either glucose (−Glc) or glutamine (−Gln) (n=3) (ns, not significant; **p<0.005; ***p<0.0005). FIG. 5G shows representative images of GLUT1 IHC staining in Ctrl and shY/T SN12C tumors (scale bar, 50 μm). FIG. 5H shows WB analysis of Ctrl and shY/T SN12C cells after being grown for 24 h in medium containing both glucose and glutamine (++) or deprived of either glucose (−Glc) or glutamine (−Gln) with indicated antibodies (ACTIN was used as loading control). FIG. 5I shows representative IF images of GLUT1 in shY/T and shY/T+MyrAKT SN12C cells (scale, 25 μm). FIGS. 5J and 5K shows change from baseline ECAR following injections of glucose (5J) or oligomycin (5K) of Ctrl, shY/T, and shY/T SN12C cells stably expressing MyrAKT1 (shY/T+MyrAKT1) (n=6) (*p<0.05; **p<0.005; ***p<0.0005). FIG. 5L shows percent change in fluorescence of Ctrl, shY/T, and shY/T+MyrAKT1 SN12C cells after being grown for 3 days in medium containing both glucose and glutamine (++) or deprived of either glucose (−Glc) or glutamine (−Gln) (n=3) (ns, not significant; ***p<0.0005). FIG. 5M shows a schematic illustrating a working model based on the results so far of how YAP/TAZ promote glycolysis.

FIG. 6 provides results from the Example relating to how YAP/TAZ can inhibit mitochondria respiratory capacity and ROS production independent of RTK-AKT signaling and mitochondrial biogenesis. FIG. 6A shows percent change in fluorescence of Ctrl and shY/T SN12C cells after grown for 3 days with or without EGF supplement in medium deprived of either glucose (—Glc) or glutamine (—Gln) (n=3) (ns: not significant; data represent mean±SD). FIG. 6B shows percent change in fluorescence of Ctrl SN12C cells after 3 days of treatment with indicated RTK inhibitors in medium with (++) or without glutamine (—Gln) (n=3) (ns: not significant; *P<0.05; **P<0.005; ***P<0.0005; data represent mean±SD). FIG. 6C shows luminescence readings (RLU) from ATP-Glo Assay of Ctrl and shY/T SN12C cells (n=3) (***P<0.0005; data represent mean±SD). FIG. 6D shows ATP levels in Ctrl and shY/T SC4 cells as measured by LC-MS/MS (n=6) (***P<0.0005; data represent mean±SD). FIGS. 6E and 6F shows representative flow cytometry profiles and quantification of Ctrl, shY/T, and shY/T+TAZ SN12C cells stained with CellROX Deep Red (6E) or MitoTracker Deep Red FM (6F) (ns=not significant; ***P<0.0005). FIG. 6G shows median fluorescence (Fluor) intensity of Ctrl and shY/T SC4 cells (n=3) stained with CellROX Deep Red or MitoTracker Deep Red FM (*P<0.05). FIG. 6H shows PCR analysis of total DNA extracted from Ctrl and shY/T SN12C cells with primers specifically targeting mitochondrial (mt) or genomic (nuc) DNA. FIGS. 6I and 6J show representative flow cytometry profiles and quantification of Ctrl, shY/T, and shY/T+AKT1 SN12C cells stained with MitoTracker Deep Red FM (6I) or CellROX Deep Red (6J) (ns=not significant; **P<0.005; ***P<0.0005). FIG. 6K shows OCRs of Ctrl and shY/T SN12C cells before or after indicated treatments (n=6) (***P<0.0005; data represent mean±SD).

FIG. 7 provides results from the Example relating to how YAP/TAZ-depleted NF2 mutant tumor cells can rely on non-canonical activation of the RAF-MEK-ERK pathway for survival. FIG. 7A shows WB analysis with indicated antibodies of shY/T SN12C cells treated with Dox for indicated days (VINC was used as loading control). FIG. 7B shows WB analysis with indicated antibodies of Ctrl and shY/T SC4 cells after 4 days of Dox treatment (VINC was used as loading control). FIG. 7C shows percent change in fluorescence of Ctrl and shY/T SN12C cells after grown for 3 days with or without Trametinib in medium containing both glucose and glutamine (++), or deprived of either glucose (—Glc) or glutamine (—Gln) (n=3) (ns: not significant; *P<0.05; **P<0.005; ***P<0.0005; data represent mean±SD). FIG. 7D shows heat map depicting IC50 values of Ctrl and shY/T SC4 cells treated for 3 days with the indicated inhibitors. FIG. 7E, 7F, and 7G shows WB analysis of pERK and pAKT levels in Ctrl and shY/T SN12C cells treated overnight with the indicated inhibitors (VINC was used as loading control; compounds that inhibited pERK but not pAKT were highlighted in Red; compounds that inhibited pAKT but not pERK was highlighted in Blue; compounds that inhibited both was highlighted in Purple). FIG. 7H shows WB analysis of pERK and pAKT levels in Ctrl and shY/T SN12C cells treated overnight with DMSO (−) or 0.1, 1, or 2 mM PKC412 (VINC used as loading control). FIG. 7I shows WB analysis of pERK and pAKT levels in Ctrl and shY/T SN12C cells treated overnight with DMSO control (−), or 1, 10, or 20 μM H-89 (samples separated by dashed-lines were run on the same blots; VINC was used as loading control). FIG. 7J shows WB analysis of pERK and pAKT levels in Ctrl and shY/T SN12C cells treated overnight with DMSO (−) or KH7 (+) (VINC used as loading control). FIG. 7K shows WB analysis of pERK and pAKT levels in Ctrl SN12C cells treated for 2 hours with 0, 0.5, 1, or 2 mM CaCl₂ (VINC was used as loading control).

FIG. 8 provides results from the Example relating to how YAP/TAZ silencing can upregulate cytosolic pH and calcium levels and cAMP-PKA/EPAC signaling, leading to noncanonical activation and increased dependency on the RAF-MEK-ERK pathway. FIG. 8A shows percent of viability of Ctrl and shY/T SN12C cells treated for 3 days with the increasing concentrations of MEK inhibitor trametinib (left) or pan-RAF inhibitor LY3009120 (right) compared to vehicle control (n=4). FIG. 8B shows heatmap depicting IC50 values of Ctrl and shY/T SC4 cells treated for 3 days with the indicated inhibitors. FIG. 8C shows WB analysis of Ctrl and shY/T SN12C cells after being grown for 24 h in medium containing both glucose and glutamine (++) or deprived of either glucose (−Glc) or glutamine (−Gln) using antibodies as indicated (ACTIN was used as loading control). FIG. 8D shows WB analysis of pERK or pAKT levels in Ctrl and shY/T SN12C cells treated overnight with DMSO control or indicated inhibitors (samples separated by dashed lines were run on the same blots; VINCULIN (VINC) was used as loading control). FIG. 8E shows WB analysis of pERK levels in shY/T SN12C cells treated overnight with DMSO control (−), trametinib, or inhibitors targeting GPCRs (lanes 3-6), soluble adenyl cyclase (lane 7), or PKA/EPAC (lanes 8-10) (VINC was used as loading control. SCH, SCH 2020676; Sot, Sotalol; GRA-1, Glucagon Receptor Agonist 1). FIG. 8F shows WB analysis of pERK or pAKT levels in Ctrl and shY/T SN12C cells treated overnight with Rp-cAMP (+) or vehicle control (−) (VINC was used as loading control). FIG. 8G shows media pH from Ctrl and shY/T SN12C cells grown for 2 days without NaHCO₃ supplement (***p<0.0005). FIG. 8H shows intracellular calcium concentration (con) in Ctrl and shY/T SN12C cells (***p<0.0005). FIGS. 8I, 8J, and 8K show WB analysis of pERK levels in shY/T SN12C cells treated for 1 h with 0, 3, 6, or 12mM HCl (8I), or for 3 h with 0, 1.6, 6.5, or 13 μM calcium chelator BAPTA (8J), or overnight with vehicle control or indicated compounds (8K) (VINC was used as loading control). FIG. 8L shows a schematic illustrating the signaling cascade induced by YAP/TAZ knockdown (KD) that causes noncanonical activation of the pro-survival RAF-MEK-ERK pathway (compounds in purple indicate the inhibitors used to delineate this signaling pathway).

FIG. 9 provides results from the Example relating to how YAP/TAZ knockdown can induce lysosomal biogenesis, which is necessary for survival under nutrient deprived conditions. FIG. 9A and 9B show WB analysis of pERK levels in shY/T SN12C cells treated for 3 hours with mitochondrial inhibitors targeting different components of the electron transport chain (9A) or (3-oxidation (9B) (VINC was used as loading control). FIG. 9C shows gene set enrichment analysis comparing genes downregulated in Ctrl relative to shY/T SN12C cells with the KEGG_Lysosome gene set. FIG. 9D shows representative images (left) and quantification (right) of Lamp1 IF staining in RFP-labeled Ctrl and shY/T SC4 cells (**P<0.005; scale=10 μm; data represent mean±SD). FIG. 9E shows representative images (left) and quantification (right) of acridine orange (AO) staining of Ctrl and shY/T SN12C cells following >8 days of Dox treatment (***P<0.0005; scale=25 μm; data represent mean±SD). FIG. 9F shows representative flow cytometry profiles of shY/T and shY/T+YAP SN12C cells stained with LysoBrite Blue. FIG. 9G shows percent viability of Ctrl and shY/T SN12C cells after grown for 24 hours in medium containing increasing concentrations of Bafilomycin compared to vehicle control (n=3) (ns: not significant; ***P<0.0005; data represent mean±SD). FIG. 9H shows percent change in fluorescence of Ctrl and shY/T SN12C cells after grown for 3 days with or without chloroquine (CQ) in medium containing both glucose and glutamine (++), or deprived of either glucose (—Glc) or glutamine (—Gln) (n=3) (ns: not significant; *P<0.05; **P<0.005; ***P<0.0005; data represent mean±SD). FIG. 91 shows representative images (left) and quantification (right) of LAMP1 IF staining in RFP-labeled shY/T SN12C cells treated with Trametinib or vehicle control (ns: not significant; scale=12.5 μm; data represent mean±SD).

FIG. 10 provides results from the Example relating to how NF2-mutant tumor cells adapt to YAP/TAZ depletion through lysosomal biogenesis and ERK activation. FIG. 10A shows heatmap depicting the relative mRNA expression of indicated lysosomal genes in Ctrl and shY/T SN12C cells as determined by microarray analysis (n=3). FIG. 10B shows representative images (left) and quantification (right) of IF staining for LAMP1 (green) and DAPI (blue) in Ctrl and shY/T SN12C cells after 2, 4, 5, or 8 days of Dox treatment (ns, not significant; **p<0.005; scale, 25 μm). FIG. 10C shows representative images (left) and quantification (right) of LAMP1 IHC staining in Ctrl and shY/T SN12C tumors (*p<0.05; scale, 25 μm). FIG. 10D and 10E show media pH (10D) or intracellular calcium concentration (10E) from shY/T SN12C cell after being grown overnight with 0 or 0.3 μM bafilomycin (Baf) (***p<0.0005). FIG. 10F shows WB analysis of pERK levels in shY/T SN12C cells treated for 3 h with 0, 0.1, 0.2, 0.3, 0.4, or 0.5 μM Baf (VINC used as loading control). FIG. 10G shows WB analysis of pERK levels in shY/T SN12C cells treated for 3 h with 0.3 μM Baf and/or 25 mM NaHCO₃ as indicated (VINC was used as loading control). FIG. 10H shows growth curves of subcutaneously implanted Ctrl (n=4) and shY/T (n=6 per group) SC4 schwannomas treated with vehicle control, trametinib, and/or Dox diet (**p<0.0005; ***p<0.0005). FIG. 10I shows a schematic illustrating a working model based on our results of how YAP/TAZ silencing elevates lysosomal biogenesis, which in turn upregulates cytosolic pH and calcium levels, initiating the sAC-cAMP-PKA/EPAC-RAF-MEK-ERK signaling cascade that promotes cell survival.

FIG. 11 provides results from the Example relating to how YAP/TAZ transcription signature correlates with the metabolic states of primary RCC tumors. FIG. 11A shows a schematic illustrating the method used to generate a YAP/TAZ transcription signature gene set (left) and heatmaps depicting results from unsupervised clustering of TCGA pRCC tumors (n=287) and VHL-WT ccRCC tumors (n=96) using YAP/TAZ transcription signature (right); log2-FC in mRNA levels between Ctrl and shY/T SN12C cells were filtered through a published ranked gene list based on their expression similarities across 1,037 cell lines from Cancer Cell Line Encyclopedia (CCLE) to identify a high confidence YAP/TAZ transcription signature containing 44 genes whose expression is regulated by YAP/TAZ and most closely associated with YAP/TAZ across CCLE cell lines (dotted boxes indicate YAP/TAZ-High (Y/T-High) and YAP/TAZ-Low (Y/T-Low) sample groups used for subsequent analyses). FIGS. 11B and 11C show average Z scores of glycolysis, OXPHOS, and lysosome gene sets in Y/T-High and Y/T-low pRCC (B) and VHL-WT ccRCC (C) tumors from FIG. 11A (**p<0.005; ***p<0.0005). FIGS. 11D and 11E show Kaplan Meier survival analysis of pRCC (11D) and VHL-WT ccRCC (11E) patients from Y/T-High and Y/T-low groups from FIG. 11A.

FIG. 12 provides results from the Example relating to a correlation of the YAP/TAZ transcription signature with the NF2 genomic alternation profiles in pRCC and VHL-WT ccRCC. FIG. 12 shows NF2 mutations and copy number alterations (CNA) in pRCC and VHL-WT ccRCC Y/T-High and Y/T-low groups.

FIG. 13 shows a table listing genesets used for analysis of KIRC and KIRP datasets.

FIG. 14 shows a table listing of inhibitor targets and concentrations used in the Example.

FIG. 15 shows tables listing KIRC VHL mutation status (table 1), KIRC YAP/TAZ subgroup (table 2), and KIRP YAP/TAZ subgroups.

### DETAILED DESCRIPTION

The present invention relates to methods comprising the administration of one or more inhibitors of the YAP/TAZ pathway, and kits comprising a pharmaceutical composition of one or more inhibitors of the YAP/TAZ pathway.

The present invention is based, in part, on the unexpected discovery that the use of an inhibitor of the YAP/TAZ pathway is effective in shrinking NF2-deficient tumors, and that inhibition of the YAP/TAZ pathway impedes the use of glucose in cancer cells, forcing cells to use their own mitochondria for energy production. As a result, the mitochondria in the NF2 cancer cells became dysfunctional upon YAP/TAZ inhibition and produces a lot of oxidative stress that damages the tumor cells, shrinking them and shutting down growth.

### Inhibitors of the YAP/TAZ Pathway

YAP, also known as YAP1 or YAP65, and TAZ are the main effectors of the Hippo tumor suppressor pathway. When the pathway is activated, YAP and TAZ are phosphorylated on a serine residue and sequestered in the cytoplasm by 14-3-3 proteins. When the Hippo pathway is not activated, YAP/TAZ enter the nucleus and regulate gene expression. Several genes are regulated by YAP, including Birc2, Birc5, connective tissue growth factor (CTGF), Amphiregulin (AREG), Cyr61, Hoxa1 and Hoxc13.

Inhibitors of the YAP/TAZ pathway may comprise an antagonist of a target protein, i.e., YAP or TAZ. As used herein, “antagonist” refers to an agent that inhibits function or activity, e.g., inhibits the function or activity of the target protein. In some embodiments, the antagonist includes an antagonist of a molecule downstream of the target protein. Suitable antagonists include an antibody or fragment thereof, a binding protein, a polypeptide, and any combination thereof. In some embodiments, the antagonist comprises a nucleic acid molecule. Suitable nucleic acid molecules include double stranded ribonucleic acid (dsRNA), small hairpin RNA or short hairpin RNA (shRNA), small interfering RNA (siRNA), or antisense RNA, or any portion thereof. In some embodiments, the antagonist comprises an optimized monoclonal antibody of the target protein.

In certain embodiments, the inhibitor of the YAP/TAZ pathway may be selected from verteporfin, (R)-PFI 2 hydrochloride, CA3 (CAS Registry Number 300802-28-2; 2,7-bis(piperidinosulfonyl)-9H-fluoren-9-one oxime; also known as CIL56), dasatinib, statins, pazopanib, β-adrenergic receptor agonists, dobutamine, latrunculin A, latrunculin B, cytochalasin D, actin inhibitors, drugs that act on the cytoskeleton, blebbistatitin, botulinum toxin C3, RHO kinase-targeting drugs (e.g., Y27632), and a combination thereof. In certain embodiments, the inhibitor of the YAP/TAZ pathway may be a compound as set forth in U.S. Patent Publication No. 2018/0297964, which is incorporated herein by reference.

Examples of statins for use in the present invention include, but are not limited to, atorvastatin, fluvastatin, lovastatin, pravastatin, rosuvastatin, simvastatin, and pitavastatin.

### Pharmaceutical Compositions

The inhibitor of the YAP/TAZ pathway may be formulated in pharmaceutical composition comprising the inhibitor of the YAP/TAZ pathway and one or more pharmaceutically acceptable excipients.

Compositions of the present invention include those suitable for oral/nasal, topical, parenteral, intravaginal and/or rectal administration. The compositions may conveniently be presented in a unit dosage form and may be prepared by any methods well known in the art of pharmacy. The amount of the inhibitor of the YAP/TAZ pathway that can be combined with a carrier material to produce a single dosage form will vary depending upon the host being treated and the particular route of administration. The amount of the inhibitor of the YAP/TAZ pathway which can be combined with a carrier material to produce a single dosage form will generally be that amount of the compound which produces a therapeutic effect.

Compositions of the present invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the inhibitor of the YAP/TAZ pathway.

In solid dosage forms for oral administration (e.g., capsules, tablets, pills, dragees, powders, granules, and the like, including for use in foods such as gum, gummy candy, as examples), the inhibitor of the YAP/TAZ pathway may be combined with one or more pharmaceutically acceptable carriers, such as sodium citrate or dicalcium phosphate, and/or any of the following: (a) fillers or extenders, such as starches, lactose, sucrose, glucose, mannitol, silicic acid, or mixtures thereof; (b) binders, such as, for example, alginates, gelatin, acacia , sucrose, various celluloses, cross-linked polyvinylpyrrolidone, microcrystalline cellulose (e.g., AVICEL® PH-101, AVICEL® PH-102), silicified microcrystalline cellulose (e.g., PROSOLV® SMCC), carboxymethylcellulose, or mixtures thereof, (c) humectants, such as glycerol; (d) disintegrating agents, such as agar-agar, calcium carbonate, alginic acid, certain silicates, sodium carbonate, sodium starch glycolate, lightly crosslinked polyvinyl pyrrolidone, corn starch, potato starch, maize starch, croscarmellose sodium, cross-povidone, or mixtures thereof; (e) solution retarding agents, such as paraffin; (f) absorption accelerators, such as quaternary ammonium compounds; (g) wetting agents, such as, for example, cetyl alcohol, glycerol monostearate, or poloxamers such as poloxamer 407 (e.g., PLURONIC® F-127) or poloxamer 188 (e.g., PLURONIC® F-68), or mixtures thereof; (h) absorbents, such as kaolin and bentonite clay; (i) lubricants, such a talc, calcium stearate, magnesium stearate, solid polyethylene glycols, sodium lauryl sulfate, colloidal silicon dioxide (i.e., hydrophobic colloidal silica, such as) AEROSIL® , stearic acid, silica gel, or mixtures thereof; and (j) coloring agents. In the case of capsules, tablets and pills, the pharmaceutical compositions may also comprise a buffering agent, such as, but not limited to, triethylamine, meglumine, diethanolamine, ammonium acetate, arginine, lysine, histidine, a phosphate buffer (e.g., sodium phosphate tribasic, sodium phosphate dibasic, sodium phosphate monobasic, or o-phosphoric acid), sodium bicarbonate, a Britton-Robinson buffer, a Tris buffer (containing Tris(hydroxymethyl)aminomethane), a HEPES buffer (containing N-(2-hydroxyethyl)piperazine-N′-(2-ethanesulfonic acid), acetate, a citrate buffer (e.g., citric acid, citric acid anhydrous, citrate monobasic, citrate dibasic, citrate tribasic, citrate salt), ascorbate, glycine, glutamate, lactate, malate, formate, sulfate, and mixtures thereof. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the inhibitor of the YAP/TAZ pathway include pharmaceutically acceptable emulsions, microemulsions, solutions, suspensions, syrups, and elixirs. In addition to the inhibitor of the YAP/TAZ pathway, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents including those listed herein, emulsifying and suspending agents, sweetening, flavoring, coloring, perfuming, and preservative agents.

Suspensions, in addition to the inhibitor of the YAP/TAZ pathway, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, methods of the invention can be administered topically, either to skin or to mucosal membranes such as those on the cervix and vagina. The topical formulations may comprise the excipients described for the solid and liquid composition set forth above, and may further include one or more of the wide variety of agents known to be effective as skin or stratum corneum penetration enhancers. Examples of such agents include 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art, e.g., salicylic acid and sulfur, may also be included.

Dosage forms for the topical or transdermal administration of the inhibitor of the YAP/TAZ pathway may include powders, sprays, ointments, pastes, creams, lotions, gels, solutions, patches, and inhalants. The inhibitor of the YAP/TAZ pathway may be mixed under sterile conditions with a pharmaceutically acceptable carrier, and with any preservatives, buffers, or propellants which may be required. The ointments, pastes, creams and gels may contain, in addition to the inhibitor of the YAP/TAZ pathway, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to inhibitor of the YAP/TAZ pathway, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise the inhibitor of the YAP/TAZ pathway in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of antioxidants that that may be used in the pharmaceutical compositions of the present invention include, but are not limited to, acetylcysteine, ascorbyl palmitate, butylated hydroxyanisole, butylated hydroxytoluene, monothioglycerol, potassium nitrate, sodium ascorbate, sodium formaldehyde sulfoxylate, sodium metabisulfite, sodium bisulfite, vitamin E or a derivative thereof, propyl gallate, edetate (e.g., disodium edetate), diethylenetriaminepentaacetic acid, bismuth sodium triglycollamate, or a combination thereof. Antioxidants may also comprise amino acids such as methionine, histidine, cysteine and those carrying a charged side chain, such as arginine, lysine, aspartic acid, and glutamic acid. Any stereoisomer (e.g., 1-, d-, or a combination thereof) of any particular amino acid (e.g., methionine, histidine, arginine, lysine, isoleucine, aspartic acid, tryptophan, threonine and combinations thereof) or combinations of these stereoisomers, may be present so long as the amino acid is present either in its free base form or its salt form.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Surfactants that that may be used in the pharmaceutical compositions of the present invention may include, but are not limited to, sodium lauryl sulfate, dioctyl sodium sulfosuccinate, dioctyl sodium sulfonate, benzalkonium chloride, benzethonium chloride, lauromacrogol 400, polyoxyl 40 stearate, polyoxyethylene hydrogenated castor oil (e.g., polyoxyethylene hydrogenated castor oil 10, 50, or 60), glycerol monostearate, polysorbate (e.g., polysorbate 40, 60, 65 or 80), sucrose fatty acid ester, methyl cellulose, polyalcohols and ethoxylated polyalcohols, thiols (e.g., mercaptans) and derivatives, poloxamers, polyethylene glycol-fatty acid esters (e.g., KOLLIPHOR® RH40, KOLLIPHOR® EL), lecithins, and mixtures thereof

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of the inhibitor of the YAP/TAZ pathway in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Compositions of the inhibitor of the YAP/TAZ pathway for intravaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Optionally, such compositions suitable for vaginal administration also include pessaries, tampons, creams, gels, pastes, foams or spray formulations containing such carriers as are known in the art to be appropriate. In some embodiments, the compositions may be suitable for use with devices such as vaginal or cervical rings.

Compositions of the present invention, including those used for oral/nasal, topical, parenteral, intravaginal and/or rectal administration may further comprise one or more pH-adjusting agents. Such pH-adjusting agents include pharmaceutically acceptable acids or bases. For example, acids may include, but are not limited to, one or more inorganic mineral acids such as hydrochloric, hydrobromic, sulfuric, phosphoric, nitric, and the like; or one or more organic acids such as acetic, succinic, tartaric, ascorbic, citric, glutamic, benzoic, methanesulfonic, ethanesulfonic, trifluoroacetic, and the like. Bases may be one or more inorganic bases or organic bases, including, but not limited to, alkaline carbonate, alkaline bicarbonate, alkaline earth metal carbonate, alkaline hydroxide, alkaline earth metal hydroxide, or amine. For example, the inorganic or organic base may be an alkaline hydroxide such as lithium hydroxide, potassium hydroxide, cesium hydroxide, sodium hydroxide, or the like; an alkaline carbonate such as calcium carbonate, sodium carbonate, or the like; or an alkaline bicarbonate such as sodium bicarbonate, or the like; the organic base may also be sodium acetate.

The pharmaceutical compositions of the present invention may be prepared using methods known in the art. For example, the inhibitor of the YAP/TAZ pathway and the one or more pharmaceutically acceptable excipients may be mixed by simple mixing, or may be mixed with a mixing device continuously, periodically, or a combination thereof. Examples of mixing devices may include, but are not limited to, a magnetic stirrer, shaker, a paddle mixer, homogenizer, and any combination thereof.

### Treatments Using Inhibitors of the YAP/TAZ Pathway

An aspect of the present invention relates to the use of inhibitors of the YAP/TAZ pathway to treat or prevent cancer. Some embodiments relate to a method of treating or preventing cancer in a subject in need thereof, the methods comprising administering one or more inhibitors of the YAP/TAZ pathway to the subject. Some embodiments relate to the use of one or more inhibitors of the YAP/TAZ pathway for treating or preventing cancer in a subject in need thereof, the use comprising administering the one or more inhibitors of the YAP/TAZ pathway to the subject. Some embodiments relate to one or more inhibitors of the YAP/TAZ pathway for use in treating or preventing cancer in a subject in need thereof, the use comprising administering the one or more inhibitors of the YAP/TAZ pathway to the subject. Some embodiments relate to a use of inhibitors of the YAP/TAZ pathway in the manufacture of a medicament for treating or preventing cancer in a subject in need thereof

In some embodiments, the cancer may be selected from the group consisting of carcinoma, sarcoma, tumors, solid tumors, blood cancer, leukemia, lymphoma, skin cancer, melanoma, breast cancer, ovarian cancer, uterine cancer, prostate cancer, testicular cancer, colorectal cancer, stomach cancer, intestinal cancer, bladder cancer, lung cancer, non-small cell lung cancer, pancreatic cancer, renal cell carcinoma, kidney cancer, liver cancer, hepatocarcinoma, brain cancer, head and neck cancer, retinal cancer, glioma, lipoma, throat cancer, thyroid cancer, neuroblastoma, endometrial cancer, myelomas, mesothelioma, and esophageal cancer.

In some embodiments, treatment or prevention of cancer may be demonstrated by one or more of the following: (i) amelioration of one or more causes or symptoms of the cancer; (ii) inhibition of one or more symptoms of the cancer from worsening; (iii) elimination of one or more symptoms of the cancer; (iv) elimination of all traces of the cancer; (v) inhibition in growth of the tumor; (vi) reduction in the size of a tumor; (vii) elimination of the tumor; (vii) inhibition of proliferation of cancer cells; (viii) inhibition of spread of cancer cells; (ix) reduction in the number of cancer cells; (x) elimination of all cancer cells; (xi) decrease in known biomarkers associated with the cancer; (xii) prevention of increase of known biomarkers associated with the cancer; (xiii) elimination of known biomarkers associated with the cancer; and (xiv) a combination thereof.

An aspect of the present invention relates to use of inhibitors of the YAP/TAZ pathway to treat or prevent noncancerous tumors or lesions. Some embodiments relate to a method of treating or preventing noncancerous tumors or lesions in a subject in need thereof, the methods comprising administering one or more inhibitors of the YAP/TAZ pathway to the subject. Some embodiments relate to the use of one or more inhibitors of the YAP/TAZ pathway for treating or preventing noncancerous tumors or lesions in a subject in need thereof, the use comprising administering the one or more inhibitors of the YAP/TAZ pathway to the subject. Some embodiments relate to one or more inhibitors of the YAP/TAZ pathway for use in treating or preventing noncancerous tumors or lesions in a subject in need thereof, the use comprising administering the one or more inhibitors of the YAP/TAZ pathway to the subject. Some embodiments relate to a use of inhibitors of the YAP/TAZ pathway in the manufacture of a medicament for treating or preventing noncancerous tumors or lesions in a subject in need thereof.

In certain embodiments, the noncancerous tumors or lesions are associated with NF2. NF2 is a is a genetic disorder marked by the predisposition to develop a variety of tumors of the central and peripheral nervous systems. The most common types of tumors associated with NF2 are vestibular schwannoma, meningioma, and ependymoma.

In some embodiments, treatment or prevention of noncancerous tumors or lesions may be demonstrated by one or more of the following: (i) amelioration of one or more causes or symptoms of the noncancerous tumors or lesions; (ii) inhibition of one or more symptoms of the noncancerous tumors or lesions from worsening; (iii) elimination of one or more symptoms of the noncancerous tumors or lesions; (iv) elimination of all traces of the noncancerous tumors or lesions; (v) inhibition in growth of the noncancerous tumors or lesions; (vi) reduction in the size of the noncancerous tumors or lesions; (vii) decrease in known biomarkers associated with the noncancerous tumors or lesions; (viii) prevention of increase of known biomarkers associated with the noncancerous tumors or lesions; (ix) elimination of known biomarkers associated with the noncancerous tumors or lesions; and (x) a combination thereof.

An aspect of the present invention relates to the use of inhibitors of the YAP/TAZ pathway to inhibit or prevent glycolysis in cancer cells. Some embodiments relate to a method of inhibiting or preventing glycolysis in cancer cells in a subject in need thereof, the method comprising administering one or more inhibitors of the YAP/TAZ pathway to the subject. Some embodiments relate to the use of one or more inhibitors of the YAP/TAZ pathway for inhibiting or preventing glycolysis in cancer cells in a subject in need thereof, the use comprising administering the one or more inhibitors of the YAP/TAZ pathway to the subject. Some embodiments relate to one or more inhibitors of the YAP/TAZ pathway for use in inhibiting or preventing glycolysis in cancer cells in a subject in need thereof, the use comprising administering the one or more inhibitors of the YAP/TAZ pathway to the subject. Some embodiments relate to a use of inhibitors of the YAP/TAZ pathway in the manufacture of a medicament for inhibiting or preventing glycolysis in cancer cells in a subject in need thereof.

In some embodiments, inhibition or prevention of glycolysis in cancer cells may be demonstrated by one or more of the following: (i) inhibition of proliferation of cancer cells; (ii) inhibition of spread of cancer cells; (iii) reduction in the number of cancer cells; (iv) elimination of all cancer cells; (v) decrease in known biomarkers associated with glycolysis in the cancer cells; (vi) prevention of increase of known biomarkers associated with glycolysis in the cancer cells; (vii) elimination of known biomarkers associated with glycolysis in the cancer cells; (viii) inhibition or decrease of expression of glycolysis genes; and (ix) a combination thereof. Glycolysis genes may include, but are not limited to, ALDOC, ENO1, ENO2, GAPDH, HK1, HK2, HK3, LDHA, PFKL, PFKP, PGK1, PGM1, SLC2A1, and SLC2A3.

An aspect of the present invention relates to the use of inhibitors of the YAP/TAZ pathway to promote or induce mitochondrial respiration in cancer cells. Some embodiments relate to a method of promoting or inducing mitochondrial respiration in cancer cells in a subject in need thereof, the method comprising administering one or more inhibitors of the YAP/TAZ pathway to the subject. Some embodiments relate to the use of one or more inhibitors of the YAP/TAZ pathway for promoting or inducing mitochondrial respiration in cancer cells in a subject in need thereof, the use comprising administering the one or more inhibitors of the YAP/TAZ pathway to the subject. Some embodiments relate to one or more inhibitors of the YAP/TAZ pathway for use in promoting or inducing mitochondrial respiration in cancer cells in a subject in need thereof, the use comprising administering the one or more inhibitors of the YAP/TAZ pathway to the subject. Some embodiments relate to a use of inhibitors of the YAP/TAZ pathway in the manufacture of a medicament for promoting or inducing mitochondrial respiration in cancer cells in a subject in need thereof.

In some embodiments, promoting or inducing mitochondrial respiration in cancer cells may be demonstrated by one or more of the following: (i) inhibition of proliferation of cancer cells; (ii) inhibition of spread of cancer cells; (iii) reduction in the number of cancer cells; (iv) elimination of all cancer cells; (v) increase in known biomarkers associated with mitochondrial respiration in the cancer cells; (vi) prevention of decrease of known biomarkers associated with mitochondrial respiration in the cancer cells; (vii) increase in mitochondrial mass in the cancer cells; (viii) increase in oxygen consumption rates in the cancer cells; and (ix) a combination thereof.

An aspect of the present invention relates to the use of inhibitors of the YAP/TAZ pathway to promote or induce oxidative stress in cancer cells. Some embodiments relate to a method of promoting or inducing oxidative stress in cancer cells in a subject in need thereof, the method comprising administering one or more inhibitors of the YAP/TAZ pathway to the subject. Some embodiments relate to the use of one or more inhibitors of the YAP/TAZ pathway for promoting or inducing oxidative stress in cancer cells in a subject in need thereof, the use comprising administering the one or more inhibitors of the YAP/TAZ pathway to the subject. Some embodiments relate to one or more inhibitors of the YAP/TAZ pathway for use in promoting or inducing oxidative stress in cancer cells in a subject in need thereof, the use comprising administering the one or more inhibitors of the YAP/TAZ pathway to the subject. Some embodiments relate to a use of inhibitors of the YAP/TAZ pathway in the manufacture of a medicament for promoting or inducing oxidative stress in cancer cells in a subject in need thereof.

In some embodiments, promoting of or inducing oxidative stress in cancer cells may be demonstrated by one or more of the following: (i) inhibition of proliferation of cancer cells; (ii) inhibition of spread of cancer cells; (iii) reduction in the number of cancer cells; (iv) elimination of all cancer cells; (v) increase in known biomarkers associated with oxidative stress in the cancer cells; (vi) prevention of decrease of known biomarkers associated with oxidative stress in the cancer cells; (vii) increase in reactive oxygen species in the cancer cells; (viii) increase in H2AX levels in the cancer cells; and (ix) a combination thereof.

Another aspect of the present invention relates to the use of inhibitors of the YAP/TAZ pathway to promote or induce lysosome-mediated activation of MAPK signaling in cancer cells. Some embodiments relate to a method of promoting or inducing lysosome-mediated activation of MAPK signaling in cancer cells in a subject in need thereof, the method comprising administering one or more inhibitors of the YAP/TAZ pathway to the subject. Some embodiments relate to the use of one or more inhibitors of the YAP/TAZ pathway for prompting or inducing lysosome-mediated activation of MAPK signaling in cancer cells in a subject in need thereof, the use comprising administering the one or more inhibitors of the YAP/TAZ pathway to the subject. Some embodiments relate to one or more inhibitors of the YAP/TAZ pathway for use in promoting or inducing lysosome-mediated activation of MAPK signaling in cancer cells in a subject in need thereof, the use comprising administering the one or more inhibitors of the YAP/TAZ pathway to the subject. Some embodiments relate to a use of inhibitors of the YAP/TAZ pathway in the manufacture of a medicament for promoting or inducing lysosome-mediated activation of MAPK signaling in cancer cells in a subject in need thereof.

In some embodiments, promoting or inducing lysosome-mediated activation of MAPK signaling in cancer cells may be demonstrated by one or more of the following: (i) inhibition of proliferation of cancer cells; (ii) inhibition of spread of cancer cells; (iii) reduction in the number of cancer cells; (iv) elimination of all cancer cells; (v) increase in known biomarkers associated with inducing lysosome-mediated activation of MAPK signaling in the cancer cells; (vi) prevention of decrease of known biomarkers associated with inducing lysosome-mediated activation of MAPK signaling in the cancer cells; (vii) increase in number of lysosomes in the cancer cells; (viii) increase in expression of lysosomal genes; and (ix) a combination thereof. Lysosomal genes may include, but are not limited to, SGSH, MAN2B1, MANBA, FUCA1, GALC, GAA, SMPD1, PSAP, PLA2G15, GBA, ASAH1, ENTPD4, DNASE2, TPP1, CTSO, CTSL1, CTSF, CTSD, CTSB, CTSA, ABCB9, AGA, SCARB2, LAMP2, LAMP1, GNPTG, GGA3, CD63, AP3M2, MCOLN1, TCIRG1, ATP6V1H, ATP6V0C, ATP6V0B, ATP6V0A2, ATP6AP1, ABCA2, and ABCA1.

In embodiments of the invention, the cancer cells are selected from the group consisting of skin cancer cells, breast cancer cells, ovarian cancer cells, uterine cancer cells, prostate cancer cells, testicular cancer cells, colorectal cancer cells, stomach cancer cells, intestinal cancer cells, bladder cancer cells, lung cancer cells, non-small cell lung cancer cells, pancreatic cancer cells, kidney cancer cells, liver cancer cells, brain cancer cells, head and neck cancer cells, retinal cancer cells, throat cancer cells, thyroid cancer cells, endometrial cancer cells, and esophageal cancer cells.

In embodiments, the inhibitor of the YAP/TAZ pathway may be administered to the subject in a therapeutically effective amount. The phrase “therapeutically effective amount”, as used in the context of inhibitors of the YAP/TAZ pathway herein, may in some embodiments refer to a quantity sufficient to elicit the biological or medical response that is being sought, including treatment of cancer, treatment of noncancerous tumors or lesions, inhibition of glycolysis in cancer cells, promotion of mitochondrial respiration in cancer cells, promotion of oxidative stress in cancer cells, and inducing lyososome-mediated activation of MAPK signaling in cancer cells.

Dosage levels of the inhibitor of the YAP/TAZ pathway may be varied so as to obtain amounts at the site of target cells (e.g., cancer cells), effective to obtain the desired therapeutic or prophylactic response. Accordingly, the therapeutically effective amount of the inhibitor of the YAP/TAZ pathway will depend on the nature and site of the target cells, the desired quantity of the inhibitor of the YAP/TAZ pathway required at the target cells to achieve the desired therapeutic or prophylactic response, the nature of the inhibitor of the YAP/TAZ pathway employed, the route of administration, the physical condition and body size of the subject, among other factors.

A therapeutically effective amount of the inhibitor of the YAP/TAZ pathway may be presented as different units. For example, a therapeutically effective amount of the inhibitor of the YAP/TAZ pathway may presented as a fixed dose. Thus, in some embodiments, a therapeutically effective amount of the inhibitor of the YAP/TAZ pathway may be about 0.1 ng to about 500 mg, or about 1 ng to about 400 mg, or about 10 ng to about 300 mg, or about 100 ng to about 200 mg, or about 1000 ng to about 100 mg; or any amount therebetween, such as about 0.1 ng, or about 0.5 ng, or about 1 ng, or about 5 ng, or about 10 ng, or about 50 ng, or about 100 ng, or about 500 ng, or about 1000 ng, or about 5000 ng, or about 0.01 mg, or about 0.05 mg, or about 0.1 mg, or about 0.5 mg, or about 1 mg, or about 5 mg, or about 10 mg, or about 50 mg, or about or about 100 mg, or about 200 mg, or about 300 mg, or about 400 mg, or about 500 mg.

A therapeutically effective amount of the inhibitor of the YAP/TAZ pathway may also be presented in units of weight of the inhibitor of the YAP/TAZ pathway per body weight of the subject. Thus, in some embodiments, a therapeutically effective amount of the inhibitor of the YAP/TAZ pathway may be about 0.1 ng to about 500 mg per kilogram of body weight (i.e., about 0.1 ng/kg to about 500 mg/kg), or about 1 ng/kg to about 400 mg/kg, or about 10 ng/kg to about 300 mg/kg, or about 100 ng/kg to about 200 mg/kg, or about 1000 ng/kg to about 100 mg/kg; or any amount therebetween, such as such as about 0.1 ng/kg, or about 0.5 ng/kg, or about 1 ng/kg, or about 5 ng/kg, or about 10 ng/kg, or about 50 ng/kg, or about 100 ng/kg, or about 500 ng/kg, or about 1000 ng/kg, or about 5000 ng/kg, or about 0.01 mg/kg, or about 0.05 mg/kg, or about 0.1 mg/kg, or about 0.5 mg/kg, or about 1 mg/kg, or about 5 mg/kg, or about 10 mg/kg, or about 50 mg/kg, or about or about 100 mg/kg, or about 200 mg/kg, or about 300 mg/kg, or about 400 mg/kg, or about 500 mg/kg.

Further, a therapeutically effective amount of the inhibitor of the YAP/TAZ pathway may be presented in units of weight of the inhibitor of the YAP/TAZ pathway per body area of the subject. Thus, in some embodiments, a therapeutically effective amount of the inhibitor of the YAP/TAZ pathway may be about 0.1 ng to about 2000 mg per square meter of the subject's body area (i.e., about 0.1 ng/m² to about 2000 mg/m²), or about 0.5 ng/m² to about 1800 mg/m², or about 1 ng/m² to about 1600 mg/m², or about 5 ng/m² to about 1400 mg/m², or about 10 ng/m² to about 1200 mg/m², or about 50 ng/m² to about 1000 mg/m², or about 100 ng/m² to about 800 mg/m², or about 500 ng/m² to about 600 mg/m², or about 1000 ng/m² to about 500 mg/m²; or any amount therebetween, such as about 0.1 ng/m², or about 0.5 ng/m², or about 1 ng/m², or about 5 ng/m², or about 10 ng/m², or about 50 ng/m², or about 100 ng/m², or about 500 ng/m², or about 1000 ng/m², or about 5000 ng/m², or about 0.01 mg/m², or about 0.05 mg/m², or about 0.1 mg/m², or about 0.5 mg/m², or about 1 mg/m², or about 5 mg/m², or about 10 mg/m², or about 50 mg/m², or about or about 100 mg/m², or about 200 mg/m², or about 300 mg/m², or about 400 mg/m², or about 500 mg/m², or about 1000 mg/m², or about 1500 mg/m², or about 2000 mg/m².

In embodiments of the invention, the inhibitor of the YAP/TAZ pathway may be administered all at once (once-daily dosing), or may be divided and administered more frequently (such as twice-per-day dosing). In some embodiments, the inhibitor of the YAP/TAZ pathway may be administered every other day, or every three days, or every four days, or every five days, or every six days, or once per week, or once per two weeks, or once every three weeks, or once every four weeks, or once every five weeks, or once every six weeks, or once every seven weeks, or once every eight weeks, or once every two months, once every three months, once every four months, once every five months, once every six months, once every seven months, once every eight months, once every nine months, once every ten months, once every eleven months, once every twelve months, once every year, or periods of time therebetween. In some embodiments, the inhibitor of the YAP/TAZ pathway may be administered as a loading dose followed by one or more maintenance doses.

In embodiments of the invention, administration of the one or more inhibitors of the YAP/TAZ pathway may be preceded by a step of identifying the subject in need thereof, i.e., identifying the subject having cancer, having cancerous lesions, having cancerous cells, etc. Such identification of the subject may be achieved by methods known in the art for diagnosing the presence of cancer, cancerous lesions, cancerous cells, etc.

### Administration of MAPK Inhibitor

In embodiments of the invention, one or more inhibitors of the YAP/TAZ pathway may be administered with one or more inhibitors of mitogen-activated protein kinase (MAPK) signaling. Administration of one or more inhibitors of MAPK signaling with the one or more inhibitors of the YAP/TAZ pathway is based in part on the discover that other signaling pathways come into play when the YAP/TAZ pathway is inhibited in tumor cells. These other signaling pathway may allow tumor cells to rewire their metabolic network to survive the new nutrient conditions, which may render them independent of the YAP/TAZ molecular pathway. Administration of an inhibitor of MAPK signaling may disrupt that cross-talk, and may provide a way to counter the resistance that YAP/TAZ-driven cancers develop to YAP/TAZ inhibiting drugs.

In some embodiments, the one or more inhibitors of MAPK signaling may comprise one or more inhibitors of the rapidly accelerated fibrosarcoma (RAF)—mitogen-activated extracellular signal-regulated kinase (MEK)—extracellular signal-regulated kinases (ERK) pathway (RAF-MEK-ERK pathway).

In certain embodiments, the one or more inhibitors of the RAF-MEK-ERK pathway may comprise one or more inhibitors of RAF.

In some embodiments, the one or more inhibitors of the RAF-MEK-ERK pathway may comprise one or more inhibitors of MEK. Examples of one or more inhibitors of MEK may include, but are not limited to, trametinib, cobimetinib, binimetinib, refametinib, selumetinib, and a combination thereof.

In certain embodiments, the one or more inhibitors of the RAF-MEK-ERK pathway may comprise an inhibitor of ERK.

The inhibitor of MAPK signaling may be administered in a same composition as the inhibitor of the YAP/TAZ pathway. Alternatively, the inhibitor of MAPK signaling may be administered concurrently in a different composition than the inhibitor of the YAP/TAZ pathway.

In some embodiments, the inhibitor of MAPK signaling may be administered before the administration of the one or more inhibitors of the YAP/TAZ pathway. Or, in certain embodiments, the inhibitor of MAPK signaling may be administered after the administration of the inhibitor of the YAP/TAZ pathway.

In some embodiments, the inhibitor of MAPK signaling may be administered shortly before, concurrently, or shortly after, the administration of the one or more inhibitors of the YAP/TAZ pathway. The term “shortly before” as used herein may mean that the inhibitor of MAPK signaling is administered to the subject about 4 hours or less, or about 3 hours or less, or about 2 hours or less, or about 1 hour or less, or about 45 minutes or less, or about 30 minutes or less, or about 15 minutes or less, prior to the administration of the one or more inhibitors of the YAP/TAZ pathway. The term “concurrently” or “concomitantly” (or other forms of these words such as “concurrent” or “concomitant”, respectively) as used herein may mean that the inhibitor of MAPK signaling is administered to the subject within about 30 minutes or less, or within about 20 minutes or less, or within about 15 minutes or less, or within about 10 minutes or less, or within about 5 minutes or less, or within about 4 minutes or less, or within about 3 minutes or less, or within about 2 minutes or less, or within about 1 minute or less, or simultaneously, of the administration of the one or more inhibitors of the YAP/TAZ pathway. The term “shortly after” as used herein means that the inhibitor of MAPK signaling is administered to the subject about 4 hours or less, or about 3 hours or less, or about 2 hours or less, or about 1 hour or less, or about 45 minutes or less, or about 30 minutes or less, or about 15 minutes or less, after the administration of the one or more inhibitors of the YAP/TAZ pathway.

In some embodiments, the one or more inhibitors of the YAP/TAZ pathway and the inhibitor of MAPK signaling are in the same pharmaceutical composition. In some embodiments, the one or more inhibitors of the YAP/TAZ pathway and the inhibitor of MAPK signaling are in different pharmaceutical compositions.

### Kits Comprising Pharmaceutical Compositions and a Package Insert

An aspect of the invention relates to kits containing one or more pharmaceutical compositions comprising one or more inhibitors of the YAP/TAZ pathway according to the present invention, and a package insert. As used herein, a “kit” is a commercial unit of sale, which may comprise a fixed number of doses of the one or more pharmaceutical compositions. By way of example only, a kit may provide a 30-day supply of dosage units of one or more fixed strengths, the kit comprising 30 dosage units, 60 dosage units, 90 dosage units, 120 dosage units, or other appropriate number according to a physician's instruction. As another example, a kit may provide a 90-day supply of dosage units.

In some embodiments, the kit may comprise a pharmaceutical composition comprising one or more inhibitors of the YAP/TAZ pathway according to the present invention, and a pharmaceutical composition comprising an inhibitor of MAPK signaling according to the present invention. In some embodiments, the kit may comprise a pharmaceutical composition comprising both one or more inhibitors of the YAP/TAZ pathway and an inhibitor of MAPK signaling according to the present invention.

As used herein, “package insert” means a document which provides information on the use of the one or more pharmaceutical compositions, safety information, and other information required by a regulatory agency. A package insert can be a physical printed document in some embodiments. Alternatively, a package insert can be made available electronically to the user, such as via the Daily Med service of the National Library of Medicines of the National Institute of Health, which provides up-to-date prescribing information. (See https://dailymed.nlm.nih.gov/dailymed/index.cfm.)

In some embodiments, the package insert may inform a user of the kit that the one or more pharmaceutical compositions may be administered according to the methods of use of the present invention.

### EXAMPLES

The following example describes a study that demonstrates that YAP/TAZ are required for the maintenance of NF2-mutant tumors.

Methods

Animal Studies

This study involved the use of a renal cell carcinoma (RCC) tumor model and a schwannoma tumor model.

For the RCC tumor model, doxycycline (Dox) inducible shRNAs against YAP and TAZ (shY/T) or a vector control (Ctrl) were stably expressed in SN12C RCC cells, which contain homozygous truncating NF2 mutations. An amount of 2×10⁵ luciferase-labeled Ctrl or shY/T SN12C cells were injected orthotopically into the renal capsule of the right kidneys of 9-10 week old SCID-Beige mice purchased from Charles River Laboratories, Burlington, MA [26]. Mice were randomly assigned to Ctrl or shY/T groups. Bioluminescent imaging (BLI) was conducted twice per week starting two weeks post-injection. Tumor-bearing mice were switched from regular to Dox-containing diet (TD0.1306; Envigo, Somerset, N.J.) when the tumor BLI flux reached the range of 3-8×10⁸ photons/second. shY/T tumors were harvested either during regression period at 3-10 days after initiating Dox treatment or at the end of life. Ctrl tumors were harvested either approximately 1-2 weeks after Dox initiation for comparison with shY/T tumors or at the end of life, respectively. Kaplan-Meier survival analysis was conducted using Prism.

For the schwannoma tumor model, 5×10⁴ pathogen-free, luciferase-labeled Ctrl or shY/T SC4 cells were injected subcutaneously into the left and right flank of 8-10 week old SCID-Beige mice purchased from Charles River Laboratories. Tumors were measured using a caliber every 2-3 days and the tumor volumes were calculated using (1*w²)/2. Once tumor volumes reached 100 mm³, mice bearing Ctrl tumors were switched to a Dox-containing diet and injected daily with 50 μ1 vehicle (5% DMSO, 1% Tween-80, 30% PEG400) via oral gavage (o.g.), whereas mice bearing shY/T tumors were randomly assigned to the following three treatment arms: (1) Dox+Vehicle (o.g.); (2) Trametinib (o.g., 2 mg/kg in vehicle); (3) Dox+Trametinib. Mice were euthanized once total tumor burden reached 2 cm or after 4 weeks of treatment. Student's t-test was used to calculate the difference in average tumor size at indicated time points.

Cell Culture

SN12C cell line was obtained from American Type Culture Collection (ATCC) and maintained in RPMI 1640 supplemented with 2 mM L-Glutamine, 10% fetal bovine serum (FBS) and penicillin/streptomycin. SC4 cell line was maintained in Dulbecco's Modified Eagle's medium (DMEM)-containing 1 mM glucose, 10% FBS and penicillin/streptomycin [27]. Unless indicated otherwise, all cells were pre-treated for 4 days with 4 μg/ml Dox prior to beginning an experiment.

Generation of Stable Cell Lines

SN12C and SC4 cells were incubated with lentiviral supernatants collected from HEK293T cells transfected with lentiviral packaging vectors together with pTripz-shYAP-RFP, pTripz-shTAZ-RFP or pTripz-RFP empty vector. shRNA sequences are as follows: YAP 5′ GTGCCACCAAGCTAGATAAAGA 3, and TAZ 5′ GGCATCTTGGTCCAGGAAATGT 3′. After 24 hours, viral supernatant was removed and cells were selected in puromycin (8 μg/ml) to eliminate uninfected cells. Successful YAP and TAZ knockdown in shY/T lines was confirmed via western blot and qRT-PCR analyses following 3-4 days of Dox treatment. To allow BLI imaging, Ctrl and shY/T SN12C cells were further infected with pHAGE-GFP-luciferase virus (Addgene plasmid #46793) produced in HEK293T as described above and sorted by flow cytometry to enrich for GFP+ cells. pLenti-CMVtight-Blast-myrAKT-HA and pLenti-CMVtight-Blast-WWTR1-HA were generated through Gateway LR reaction combining pLenti-CMVtight-Blast-DEST (w762-1) (Addgene plasmid #26434) with either pEntr-myr-AKT-HA (Addgene plasmid #31790) or pEntr223-WWTR1 (DNASU, Tempe, Ariz.). Viral supernatants for pLenti-CMVtight-Blast-myrAKT-HA, pLenti-CMVtight-Blast-WWTR1-HA, and FUW-tetO-wtYap (Addgene plasmid #84009) were generated as described above and used to infect shY/T SN12C cells, followed by selection with blasticidin (5 μg/ml ).

### Nutrient Deprivation Experiments

For SN12C cells, all medium in nutrient deprivation studies contained a base of RPMI salt mix (0.42 mM Ca(NO₃)₂.4H₂O, 0.41 mM MgSO₄, 5.4 mM KCl, 23.8 mM NaHCO₃, 102.7 mM NaCl, and 5.6 mM Na₂HPO₄), RPMI 1640 vitamins solution (Millipore Sigma, St. Louis, Mo.), MEM amino acids solution (ThermoFisher Scientific), and MEM non-essential amino acids solution (Sigma). Nutrient replete conditions received 11.1 mM glucose (Millipore Sigma) and 2 mM glutamine (Lonza, Benicia, Calif.) and deprivation conditions lacked or contain reduced amount of their respective nutrient as indicated. For SC4 cells, all medium in nutrient deprivation studies contained a base of DMEM without glucose or glutamine (#D5030, Millipore Sigma) and supplemented with sodium pyruvate and sodium bicarbonate to levels in standard DMEM formulation. For conditions with glucose or glutamine, 5.6 mM glucose (Millipore Sigma) and/or 4 mM glutamine (Lonza) were added. Reduced cellular glutathione (GSH) (Alfa Aesar, Haverhill, Mass.) and epidermal growth factor (EGF) (Millipore Sigma) were used at final concentrations of 0.3 mM and 1 μg/ml, respectively. Percent survival was calculated based on the sequential measurements of red fluorescent protein (RFP) fluorescence (filter range 553-574 nm) at the initiation and end of treatment using the Synergy™ Hybrid Multi-Mode Microplate Reader (BioTek, Winooski, Vt.). A one-way ANOVA with Sidak's multiple comparison test was used to calculate significance for all nutrient deprivation experiments.

### Hypoxia Treatment

Cells were grown for indicated times in a 37° C. cell incubator supplied with 2% O₂ (hypoxia) or 21% O₂ (normoxia). For proliferation assay, cells were trypsinized, and counted using the Z1 Coulter Particle Counter (Beckman Coulter Life Sciences, Brea, Calif.) at the start and end of the experiment. Percent survival was calculated by comparing the final cell numbers to the initial cell numbers. For western blot, cells were washed quickly with ice-cold phosphate-buffered saline (PBS) and lysed in urea buffer.

### Immunohistochemistry Analysis

Mouse kidney tumors were fixed in 10% neutral buffered formalin and paraffin-embedded sections were used for all immunohistochemistry (IHC) analyses. Unstained slides were deparaffinized, rehydrated, and heated in antigen retrieval buffer (IHC-Tek™ Epitope Retrieval Solution, IHC World LLC, Woodstock, Md. or TRIS solution (10 mM Tris Base, 1 mM EDTA Solution, 0.05% Tween 20, pH 9.0)) for 30 minutes at 95° C. Ki67 (1:200, #RM-9106, ThermoFisher Scientific), glucose transporter 1 (GLUT1) (1:5000, #07-1401, Millipore Sigma), PIMO (Hypoxyprobe, 1:500, #HP1, Hypoxyprobe, Inc.) and lysosomal-associated membrane protein 1 (LAMP1) (1:200, #9091s, CST) stained tissues received IHC-Tek™ antigen retrieval buffer and YAP (1: 25, #4912s, Cell signaling Technology, CST, Danvers, Mass.), TAZ (1:200, #4883s, CST), and pH2AX (1:100, #9718s, CST) received TRIS antigen retrieval solution. Once the slides were cooled to room temperature, they were washed twice with PBS, treated with 3% H₂O₂ in H₂O for 10 minutes, washed twice with PBST (PBS with 0.05% Tween 20), and blocked with 2% horse serum in PDT (PBS+1% Triton X-100). After removing the blocking solution, primary antibody dilutions (in PDT+2% horse serum) were added and the slides were incubated overnight at 4° C. The next day, the slides were washed 5 times in PBST and then incubated with their corresponding horseradish peroxidase (HRP)-conjugated secondary antibodies (Vector Laboratories, Burlingame, Calif.) for 1 hour at room temperature. Slides were washed 3 times with PBST and staining was visualized using ImmPACT DAB EqV HRP substrate (Vector Laboratories) according to the manufacturer's instructions. The slides were counterstained with Haematoxylin for 1 minute, washed for 5 minutes with running H₂O, dehydrated, and mounted. Finished IHC slides were scanned using the Leica SCN400 F whole-slide scanner (NYU OCS Experimental Pathology Histology Core Lab, New York, N.Y.) and images were analyzed using the Leica Digital Image Hub (Leica, Buffalo Grove, Ill.). All quantification was conducted using ImageJ software and data from at least 5 slides or fields per sample was obtained. For all quantification with 3 sample groups, one-way ANOVA using Tukey's multiple comparison test was used to calculate statistical significance.

### Annexin V/Sytox Flow Cytometry Analysis

Cells were cultured in the indicated conditions, collected after 3 days for analysis. Cells were stained according to the Annexin V staining protocol (BioLegend) and Sytox Blue (Thermo Fisher Scientific) was added immediately prior to Flow Cytometry analysis at a final concentration of 1 μM.

Glucose Uptake-Gb™ and ROS-Glo™ H₂O₂ Assays

Cells were cultured in 24-well plates for Glucose Uptake assay or 96-well plates for ROS assay and the respective experiments were conducted according to the manufacturer's instructions (Promega, Madison, Wis.). Luminescence was normalized using the average crystal violet staining absorbance of 3 replicates plated in parallel of experimental samples. For crystal violet staining, cells were fixed in 4% paraformaldehyde, stained overnight with crystal violet solution containing 0.1% crystal violet (Alfa Aesar) in 10% Ethanol, washed with distilled water until excess solution was removed, and dried at room temperature. Crystal violet absorbance was measured at 595 nm using Synergy™ Hybrid Multi-Mode Microplate Reader (BioTek). One-way ANOVA using Sidak's multiple comparison test was used to calculate significance of ROS measurements in nutrient deprivation conditions.

### Seahorse Glycolysis and Mitochondrial Stress Tests

Unless otherwise indicated, 100,000 Ctrl and shY/T cells were plated in regular growth medium on 2% Geltrex (ThermoFisher Scientific) coated 96-well Seahorse plates (Agilent Santa Clara, Calif.) and allowed to attach overnight. After a brief wash with serum-free media, the cells were incubated for additional 24 hours in serum-free media, at which point the media was replaced for an hour with glucose/glutamine/NaHCO₃-free RPMI or DMEM (for SN12C and SC4, respectively) prior to initiation of extracellular acidification rate (ECAR) measurements or with NaHCO₃-free RPMI or DMEM prior to initiation of OCR measurements. All medium was pH adjusted to 7.4 before administering. Tests were carried out according to manufacturer's instructions (Agilent) using the Seahorse XFe96 Analyzer. For glycolysis analysis, glucose (Millipore Sigma), oligomycin (Millipore Sigma), and 2-DG (TCI LAB, Riverside, Calif.) were injected sequentially at final concentrations of 11 mM, 2 μM, and 50 mM, respectively. For standard Mitochondrial Stress test, oligomycin (Millipore Sigma), carbonyl cyanide-4-(trifluoromethoxy)phenylhydrazone (FCCP) (Millipore Sigma), Rotenone (Millipore Sigma), Antimycin A (Millipore Sigma) were injected sequentially at final concentrations of 2 μM, 2 μM, 0.5 μM, and 0.5 μM, respectively. For oxidative consumption rate (OCR) measurements following injections of either Glutamine or Pyruvate, final concentrations of 2 mM and 10 mM were used, respectively. For Complex I and Complex II assays, cells were permeabilized for 1 hour at 37° C. in mitochondrial assay buffer (70 mM sucrose, 220 mM mannitol, 10 mM KH₂PO₄, 1 mM ethylene glycol-bis(β-aminoethyl ether)-N,N,N′,N′-tetraacetic acid (EGTA), and 2% [w/v] fatty acid free BSA, pH=7.4) supplemented with 10 mM adenosine diphosphate (ADP) and 50 mg/mL saponin and all injections were dissolved in this buffer [28]. To determine Complex I activities, pyruvate (Thermo Fisher Scientific), malate (Alfa Aesar), Rotenone, Atpenine A5 (Cayman Chemicals), and Antimycin A were injected sequentially at final concentrations of 10 mM, 5 mM, 0.5 μM ,1 μM, and 0.5 μM, respectively. To determine Complex II activities, Rotenone, succinate (Acros Organics), Atpenin A5, and Antimycin A were injected sequentially at final concentrations of 0.5 μM, 10 mM, 1 μM, and 0.5 μM, respectively. All raw data were normalized to total protein amounts analyzed using the Pierce BCA Protein Assay Kit (ThermoFisher Scientific).

### Microarray Analysis

Ctrl and shY/T SN12C cells grown in serum-free medium for 24 hours were extracted for total mRNA using the RNAeasy Mini kit (Qiagen, Hilden, Germany) according to the manufacturer's instructions. Total mRNA were amplified, labeled, and hybridized to the Illumina HumanHT-12 V4 Expression Array at the University of Chicago Genomics Facility, Chicago, Ill. Raw intensity data was background corrected, normalized, and differential expression was calculated using the Bioconductor limma package in R. The Broad Institute's GSEA Java-based software was used to determine enrichment scores, significance of enrichment, and enrichment plots. The heatmap was generated using excel conditional formatting based on normalized raw expression values.

### Western Blot Analysis

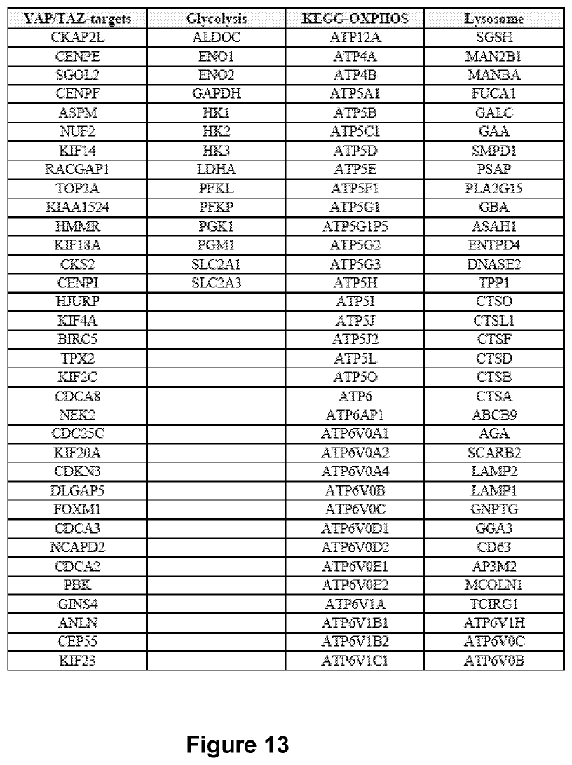

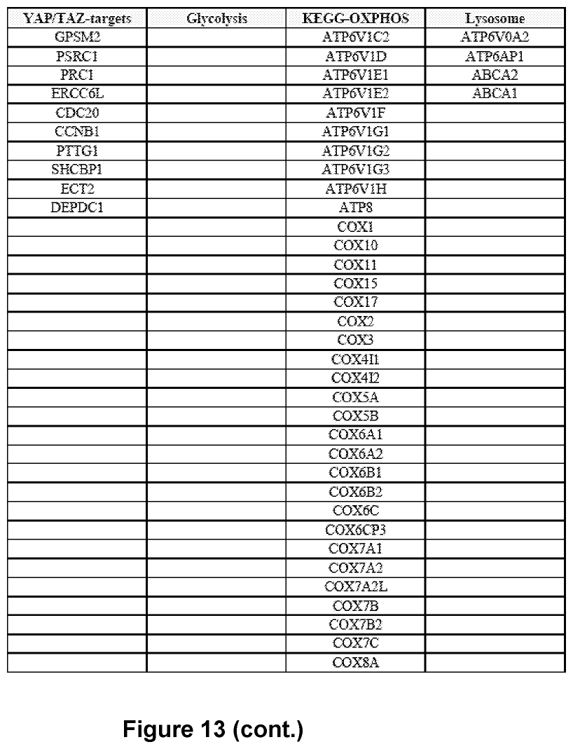

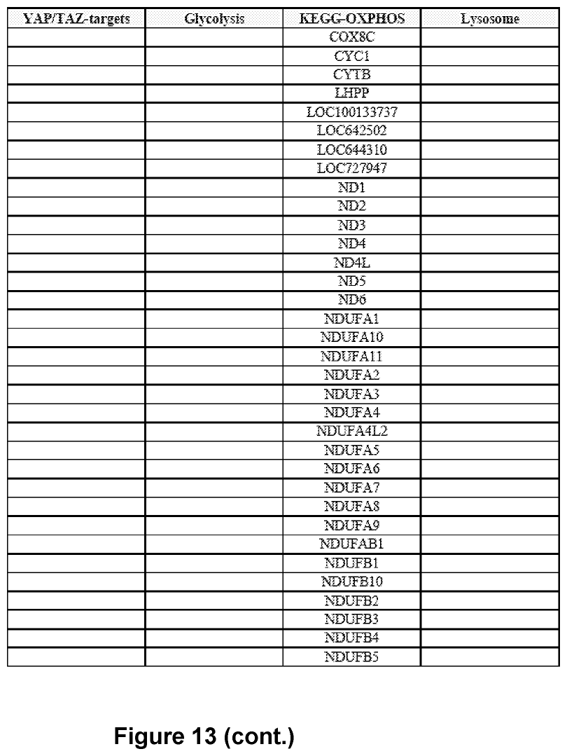

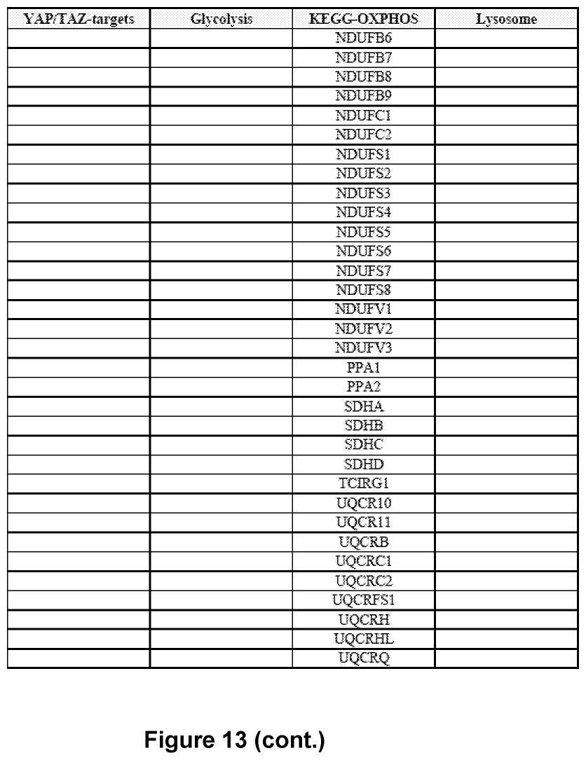

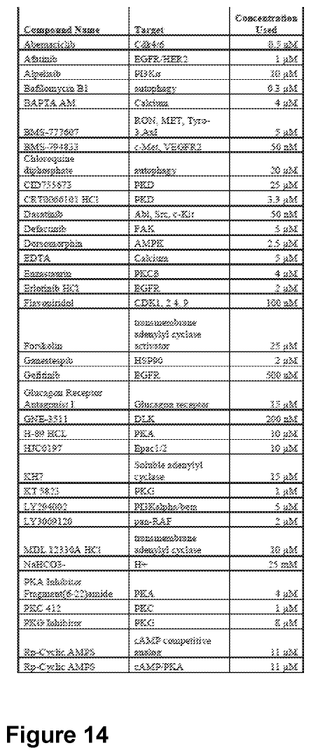

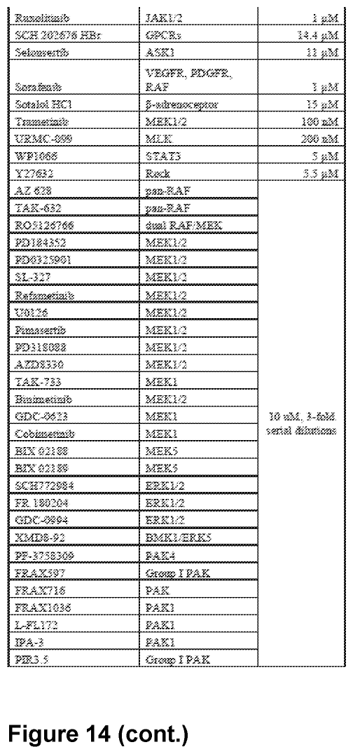

Cells were grown for 24 hours in serum-free medium in the presence of various inhibitors unless indicated otherwise. After a brief wash with cold PBS, cells were lysed in urea buffer (9.5 M urea, 2% 3-[(3-cholamidopropyl)dimethylammonio]-1-propanesulfonate (CHAPS)), adjusted to similar concentrations, mixed with 6× sodium dodecyl sulfate (SDS) loading dye, heated at 95° C. for 10 minutes, and subjected to SDS-polyacrylamide gel electrophoresis and semi-dry transferring to polyvinylidene fluoride (PVDF) membranes. For analysis of oxidative phosphorylation (OXPHOS) complexes, 2×10⁷ Ctrl and shY/T cells were harvested and either saved as whole cell extract (WCE) or processed using the Mitochondria Isolation Kit for cultured cells according to the manufacturer's instructions (Thermo Fisher Scientific). The WCE and mitochondrial-enriched fraction were resuspended in radioimmunoprecipitation assay (RIPA) buffer for western blot analysis. Primary antibodies used are listed in the table in FIG. 13. Inhibitor information can be found in the table in FIG. 14.

### LC-MS/MS Targeted Metabolomic Analysis

After 5 days of Dox pre-treatment, SC4 cells were washed twice with DMEM and incubated for additional 24 hours in Dox-containing DMEM. The next day cells were washed with ice-cold high performance liquid chromatography (HPLC) grade PBS, drained, snap-frozen in liquid nitrogen and stored at −80° C. until further processing. Metabolite extraction was conducted in 50% methanol, 30% ACN, and 20% water at 1 mL/1×10⁶ cells [29]. Samples were then vortexed for 5 minutes at 4° C. followed by centrifugation at 16,000 g for 15 minutes at 4° C. The supernatants were collected and separated by liquid chromatography-mass spectrometry using SeQuant ZIC-pHilic column (Millipore Sigma). The solvent for aqueous mobile-phase 20 mM ammonium carbonate with 0.1% ammonium hydroxide solution and the solvent for organic mobile phase was acetonitrile. To separate the metabolites, a linear gradient from 80% organic to 80% aqueous for 15 minutes with a column temperature of 48° C. and a flow rate of 200 μl/minute was used. The metabolites were detected across a mass range of 75-1,000 m/z using the Q-Exactive mass spectrometer at a resolution of 35,000 (at 200 m/z) with electrospray ionization and polarity switching mode [29]. Lock masses were used to insure mass accuracy below 5 ppm. The Thermo TraceFinder software was used to determine the peak areas of different metabolites using the exact mass of the singly charged ion and known retention time on the HPLC column.

qRT-PCR Analysis

Ctrl and shY/T SN12C cells grown for 24 hours in serum-free medium were harvested for total mRNA using the RNAeasy Mini kit (Qiagen, Hilden, Germany) according to the manufacturer's instructions. Reverse transcription was conducted using the iScript cDNA Synthesis Kit (Bio-Rad, Hercules, Calif.) and the resulting cDNA products were amplified with iTaq Universal SYBR Green Supermix (Bio-Rad) in triplicates. Gene expression fold change was calculated as a unit value of 2^{-ΔCt}=2^{[Ct(HPRT)-Ct(Gene of Interest)]]}. Data is represented by mean, minimum, and maximum of all replicates.

### CellROX Analysis

To measure total cell reactive oxygen species (ROS) levels, CellROX Deep Red reagent (ThermoFisher Scientific) was added to culture medium at a final concentration of 5 μM and incubated for 30 minutes at 37° C. Cells were then trypsinized, resuspended in culture medium, and centrifuged at 500 G for 5 minutes. The supernatant was removed and the cell pellet was resuspended in PBS, filtered through a 35 μm strainer and 30,000 cells were analyzed by flow cytometry. Data were analyzed from three independent experiments.

### MitoTracker Analysis

Cells were trypsinized, resuspended in culture medium, and centrifuged at 500G for 5 minutes. Supernatant was removed and 100 nM MitoTracker Deep Red (Thermo Fisher Scientific) in PBS was added to each sample followed by a 30 minute incubation at 37° C. Cells were centrifuged at 500 G for 5 minutes and supernatant was removed. Cells were resuspended in PBS and 30,000 cells were analyzed by flow cytometry.

### Immunofluorescence Analysis

Cells were plated on coverslips coated with a solution of 2% Geltrex (Thermo Fisher Scientific) in media and subjected to the indicated nutrient treatments for the specified time periods. Cells were then fixed in 4% paraformaldehyde for 30 minutes and washed 2 times with PBS. Next, cells were permeabilized using 0.3% Triton X-100 in PBS for 15 minutes, washed twice with PBST (PBS+0.1% Tween-20), and blocked in 5% appropriate serum in PBST. Coverslips were then incubated with primary anti-GLUT1 (#07-1401, Millipore Sigma), anti-mito (#MAB1273, Millipore Sigma), or anti-LAMP1 (#9091s, CST) antibodies diluted in PBST for 45 minutes at 37° C. After 5 washes in PBST, fluorescein-conjugated secondary antibodies diluted in PBST (1:200) were added to the coverslips and incubated for 1 hour at room temperature. Coverslips were washed again and mounted on glass slides using Fluoroshield™ with Dapi (Millipore Sigma) mounting medium. Confocal fluorescent images were acquired using Leica SP8 Confocal microscope and quantification analysis was conducted using ImageJ software.

### Electron Microscopy

Cells grown to 80% confluence on 60 mm cell culture dishes were fixed with 2.5% glutaraldehyde, 1% paraformaldehyde in 0.12 M sodium cacodylate buffer pH 7.4 for one hour at room temperature. After buffer washes, cells were fixed in 1% osmium tetroxide for one hour. Following washes, cells were en bloc stained with 1% uranyl acetate in water overnight. The cells were then dehydrated through an ethanol series and infiltrated with epoxy resin (LX112, Ladd Research Industries, Inc.). Inverted Beem capsules were placed into each tissue culture dish to create on face blockfaces for sectioning, cured for 48 hours at 60° C. The 70 nm sections were post-stained with 1% uranyl acetate and lead citrate before imaging in the FEI ThermoFisher Talos 200 KV TEM operated at 80 KV.

### Mitosox, Mitotracker, and Acridine Orange IF Analysis

Ctrl and shY/T SN12C cells were plated in normal growth media on coverslips coated with 2% Geltrex. On the next day, cells on coverslips were washed and incubated in RPMI for additional 24 hours. 5 nM Mitosox (ThermoFisher Scientific) and 50 nM MitoTracker Deep Red FM (ThermoFisher Scientific) were added to the media and incubated for 25 minutes at 37° C. For acridine orange staining, 10 μg/ml was added to the media and incubated for 20 minutes at 37° C. Cells were then washed twice with PBS, mounted on glass slides using Fluoroshield™ and immediately imaged at 63× on the Leica SP8 Confocal microscope [30, 31].

### Mitochondrial DNA Copy Number Analysis

2×10⁶ Ctrl and shY/T SN12C cells were harvested and total DNA was extracted using Prepease° Genomic DNA Isolation Kit (Affymetrix, Santa Clara, Calif.) and analyzed by RT-PCR using GoTaq® Green Master Mix (Promega) with primer sets that specifically detect nuclear (nucDNA) and mitochondrial (mtDNA). Primer sequences are as follows: nucDNA forward 5′ TGCTGTCTCCATGTTTGATGTATCT 3′ and reverse 5′ TCTCTGCTCCCCACCTCTAAGT 3′; mtDNA forward 5′ CACCCAAGAACAGGGTTTGT 3′ and reverse 5′ TGGCCATGGGTATGTTGTTA 3′. PCR conditions and sequences of the nuclear and mitochondrial primers were reported previously [32]. The final PCR products were run on 2% agarose gel and visualized by UV light.

### NAD/NADH-Glo™, NADP/NADPH-Glo™, and GSH/GSSG-Glo™ Assays

Cells grown for 24 hours in serum-free medium were processed for the NAD/NADH-Glo™, NADP/NADPH-Glo™, or GSH/GSSG-Glo™ assays (Promega) according to the manufacturer's instructions. At the time of the assay, the cell numbers of 3 replicates plated in parallel with experimental samples were counted using the Z1 Coulter Particle Counter (Beckman Coulter Life Sciences) and the average cell number was used to normalize luciferase readings.

### Drug IC-50 Studies

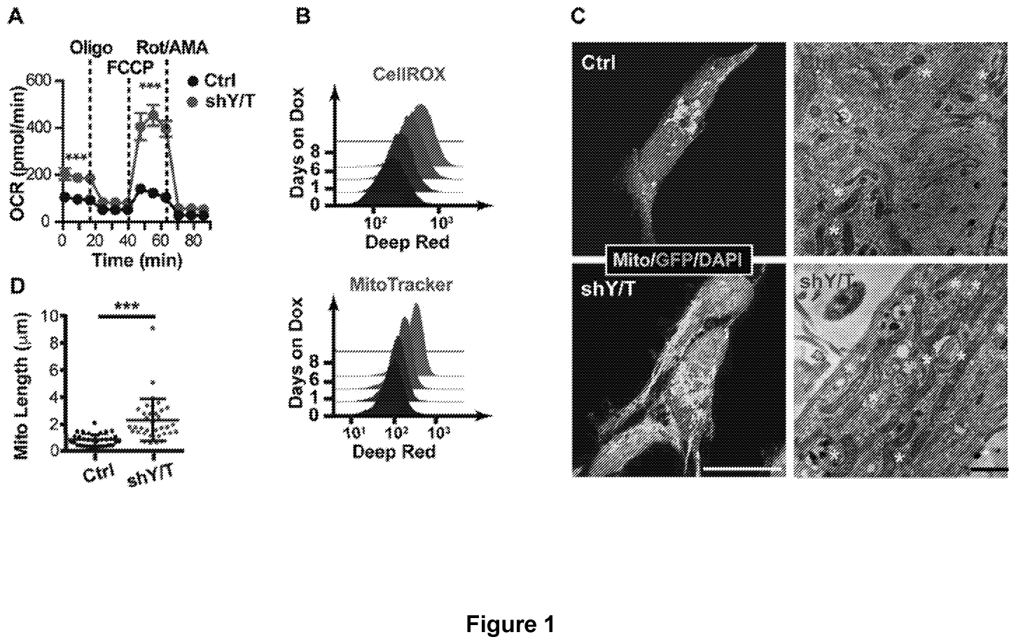

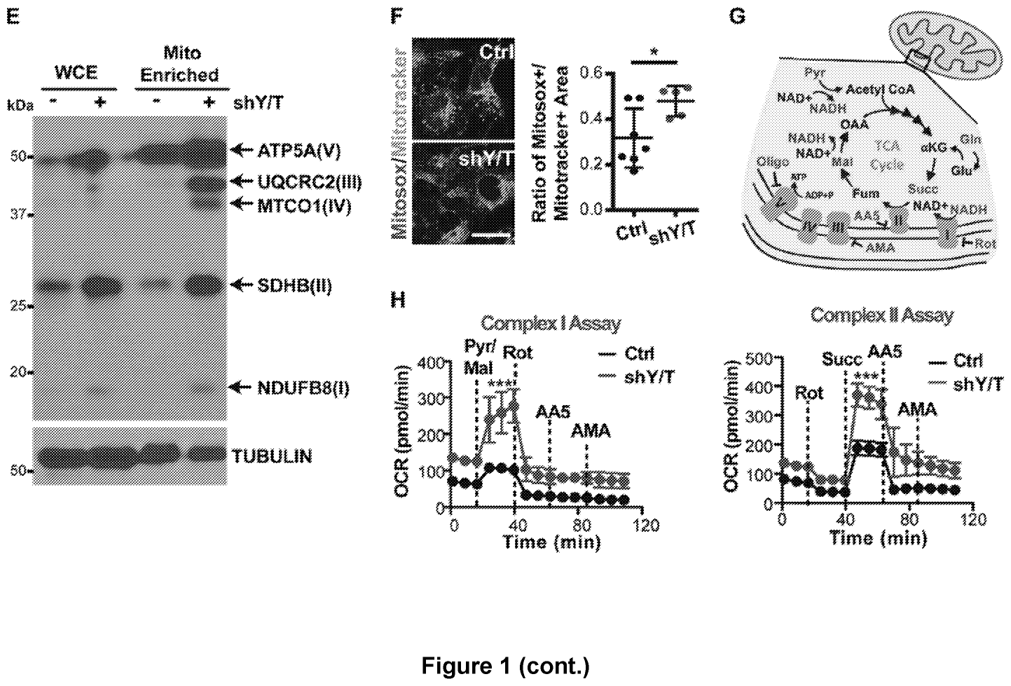

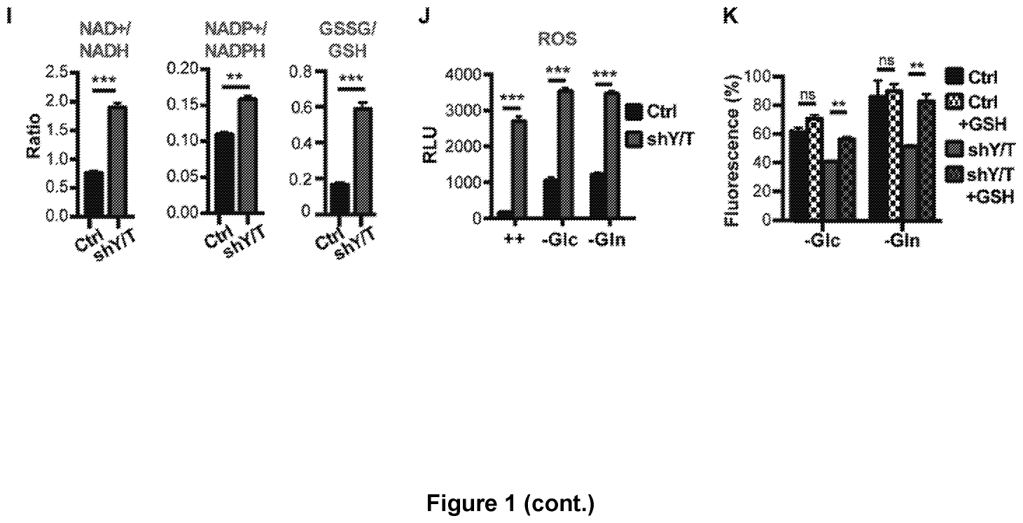

Cells were plated at optimized seeding densities to reach 60-70% confluence on the next day, at which point the media were replaced with serum-free medium containing Dox and serially diluted compounds or vehicle control. SC4 cells in FIG. 1B were plated on 384-well plates using a Microdrop™ Combi Reagent Dispenser (Thermo Fisher) and the next day serial dilutions were prepared in DMSO and added to the wells using the Janus Automated Workstation (Perkin Elmer). After incubation for indicated time, cell viability was measured using the CellTiter-Glo Luminescence Cell Viability assay (Promega) or ATPlite Luminescence Assay (Perkin Elmer) for FIG. 1B. Percent viability for each cell lines was calculated based on vehicle control.

### pH Measurements

Cells were plated at the same time with optimized seeding densities that ensure all cell lines to reach 80% confluence at the time of measurement. Medium supernatant was collected and measured using the Thermo Scientific™ Orion™ 3-Star Benchtop pH Meter after calibration.

### Intracellular Calcium Assay

Cells were grown for indicated time in serum-free medium, washed with ice-cold PBS, collected in 100 mM Tris pH 7.5 buffer using cell scraper, and processed using the Calcium Assay Kit (Cayman Chemicals) according to the manufacturer's instructions. The calcium concentrations were normalized to the protein concentrations determined using Bradford Protein Assay (Bio-Rad).

### LysoBrite Blue Analysis

Cells were treated with lx Lysobrite Blue (Cayman Chemical) for 45 minutes at 37° C. Cells were then trypsinized, resuspended in culture medium, and centrifuged at 500 G for 5 minutes and analyzed by flow cytometry.

### Correlation Analysis in Primary RCC Tumors

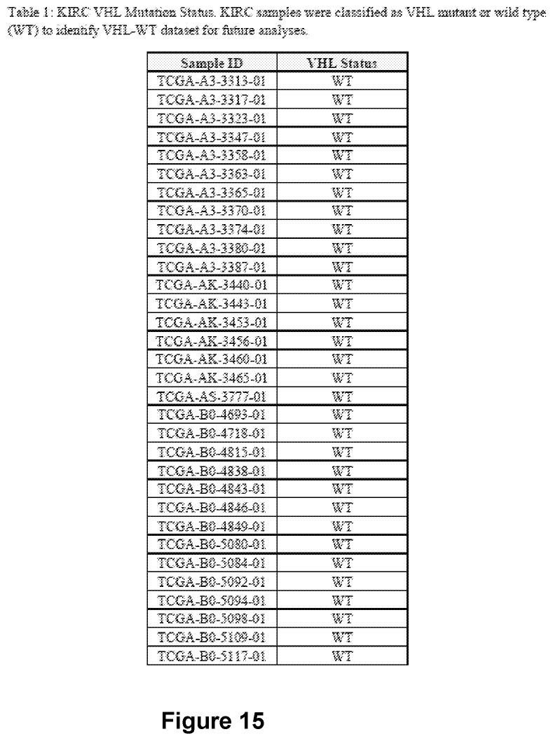

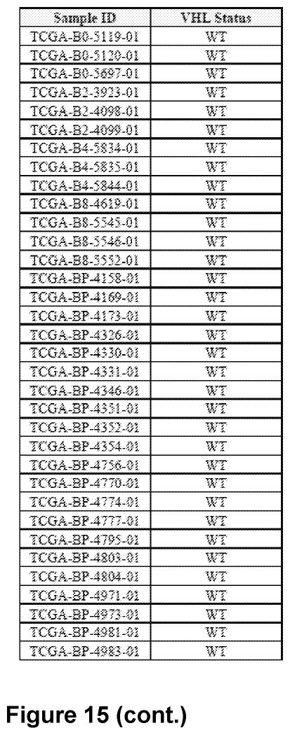

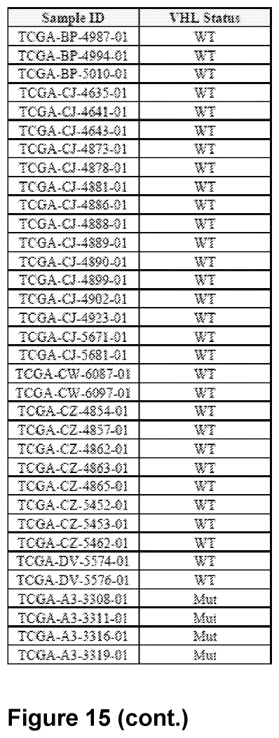

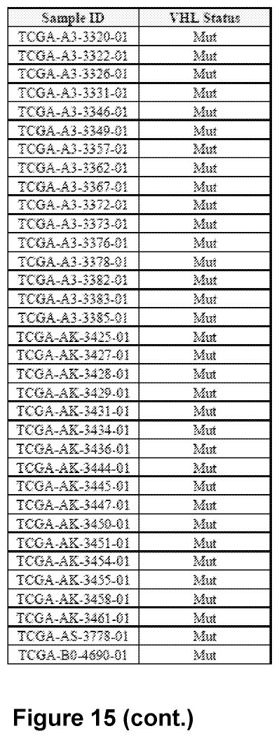

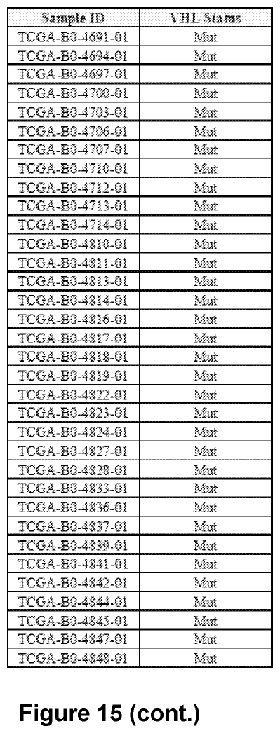

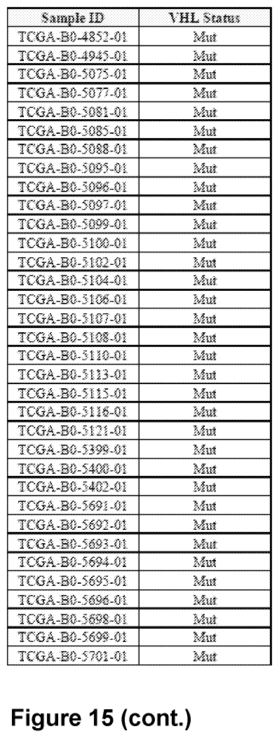

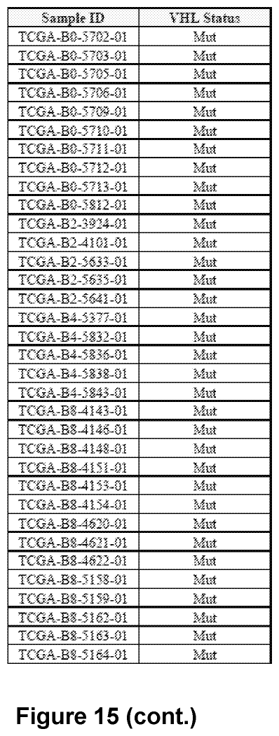

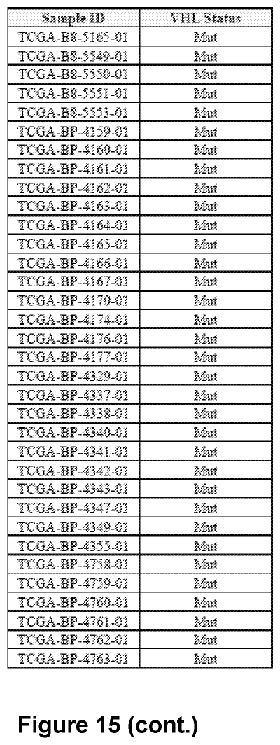

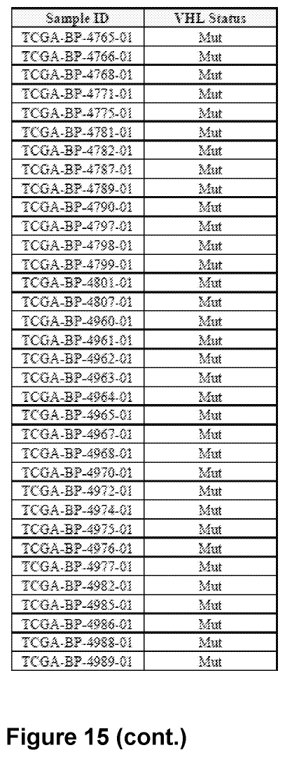

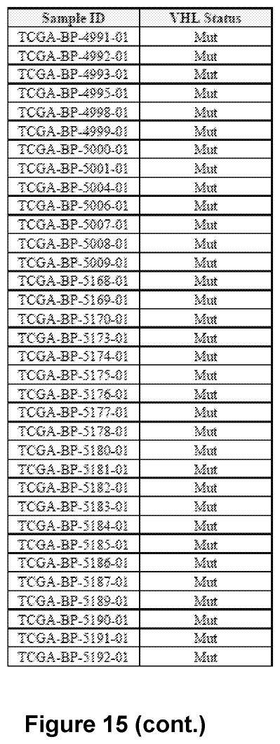

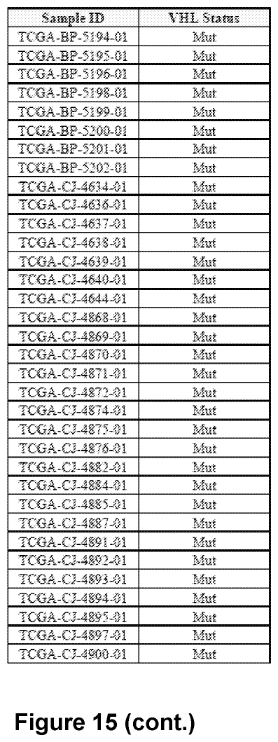

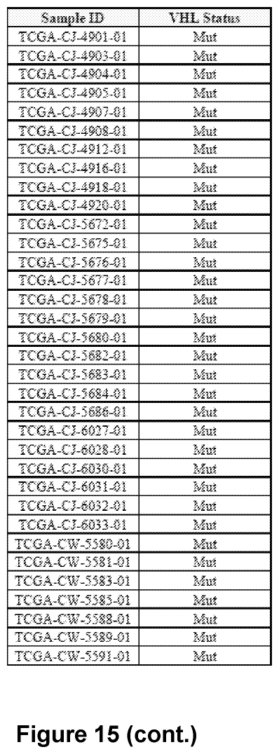

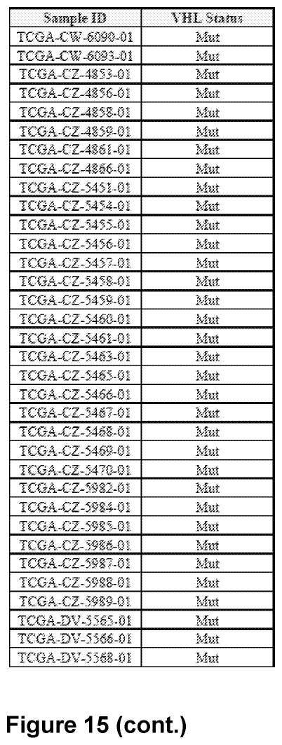

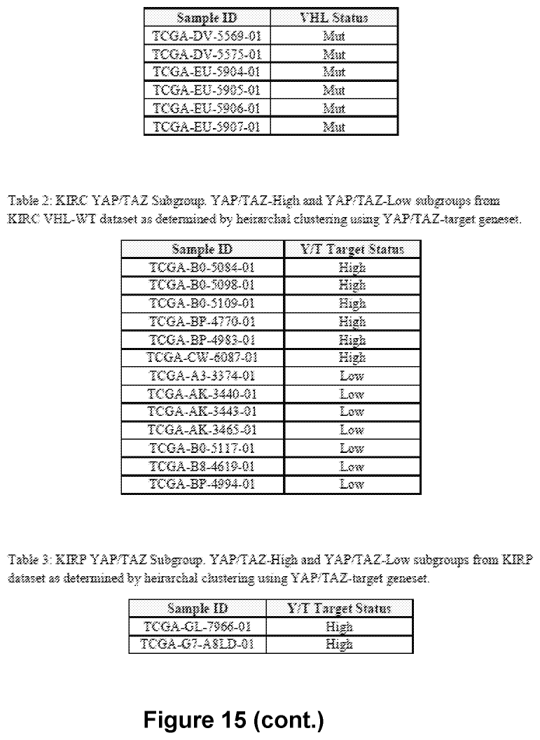

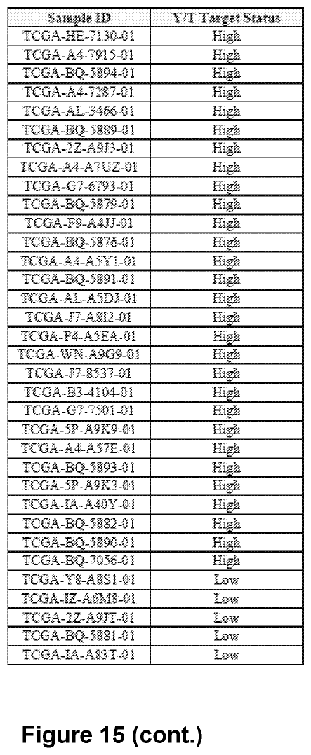

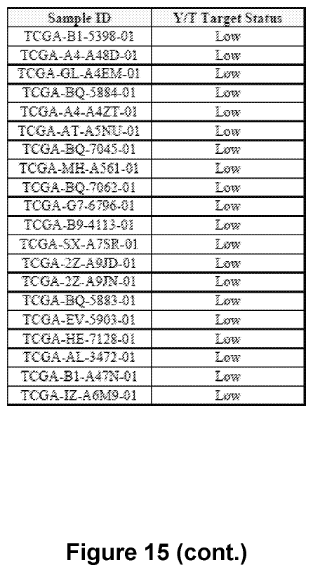

First, we used a published ranked genelist whose order is reflective of each gene's relative similarity across CCLE cell lines [33] to identify groups of gene whose expression correlates with YAP/TAZ expression by summing the log2FC of Ctrl vs shY/T determined by our microarray analysis in SN12C cells for each gene across 20-gene increments. Once we determined the placement on the ranked genelist where the Σlog2FC was the greatest, we broadened the 20-gene window to include adjacent genes with similar log2FC values. This list of 44 genes was identified as the YAP/TAZ transcription signature geneset (table in FIG. 15), which was used to stratify RNAseq data from primary ccRCC (TCGA-KIRC) and pRCC (TCGA-KIRP) tumors downloaded from the GDC Data Portal (https://portal.gdc.cancer.gov/).

For analysis of TCGA-KIRC dataset, samples were first classified as VHL-mutant or VHL-WT based on their corresponding mutation, copy number and RNAseq data, yielding 96/446 (21.5%) VHL-WT ccRCC tumors for the subsequent analyses (tables in FIG. 15). Unsupervised hierarchical clustering was conducted using the above-described YAP/TAZ transcription signature (table in FIG. 13) against 97 VHL-WT ccRCC tumors or all 287 TCGA-KIRP pRCC tumors with RNAseq data. We designated the groups with the highest and lowest expression of YAP/TAZ transcription signature from each tumor dataset as YAP/TAZ-High and YAP/TAZ-Low groups, respectively. Assessment of the relative expression of glycolysis, OXPHOS, and lysosomal genes between the YAP/TAZ-High and YAP/TAZ-Low groups was conducted by calculating the Z score for each gene within the geneset and then averaging the Z scores for each sample. Kaplan-Meier survival analysis was conducted using the TCGA_biolinks package in R.

### Quantification and Statistical Analysis

Graphpad Prism was used to conduct all statistical analysis unless otherwise stated. Statistical tests are indicated in figure legends for the respective experiments. Kaplan-Meier survival curve was used to analyze the survival differences between experimental groups. Significance was determined as a p-value of 0.05 or less. Error bars on all graphs indicate standard deviation unless stated otherwise in the figure legend. Unless stated otherwise, data for each method were analyzed from three independent experiments.

### Data and Software Availability

Microarray datasets have been deposited to NCBI's Gene Expression Omnibus. They are accessible through the accession number GEO: GSE125408.

### Results

YAP/TAZ Depletion Induces Tumor Regression and Prolongs Survival in Mice Bearing NF2-Mutant Kidney Tumors

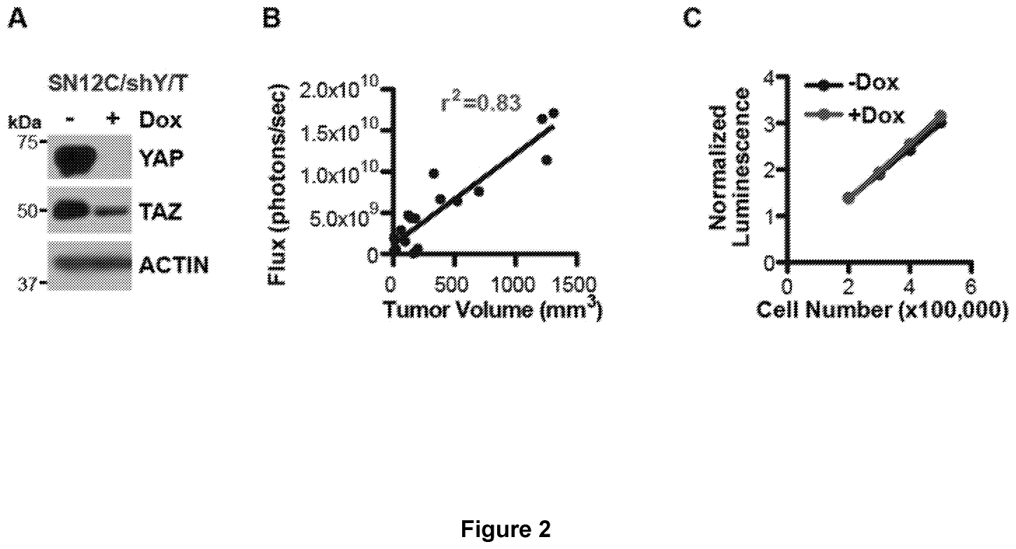

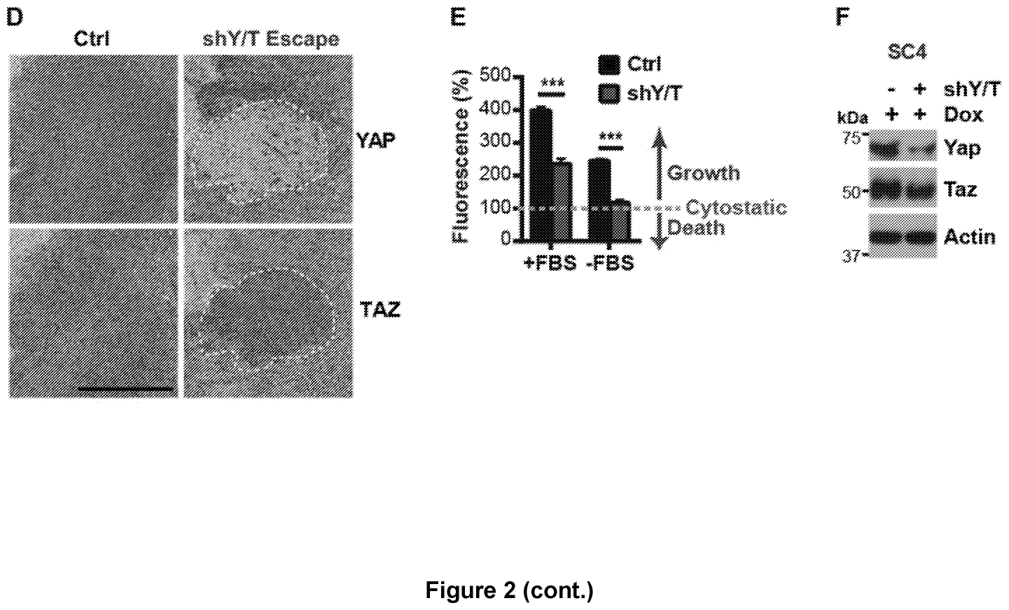

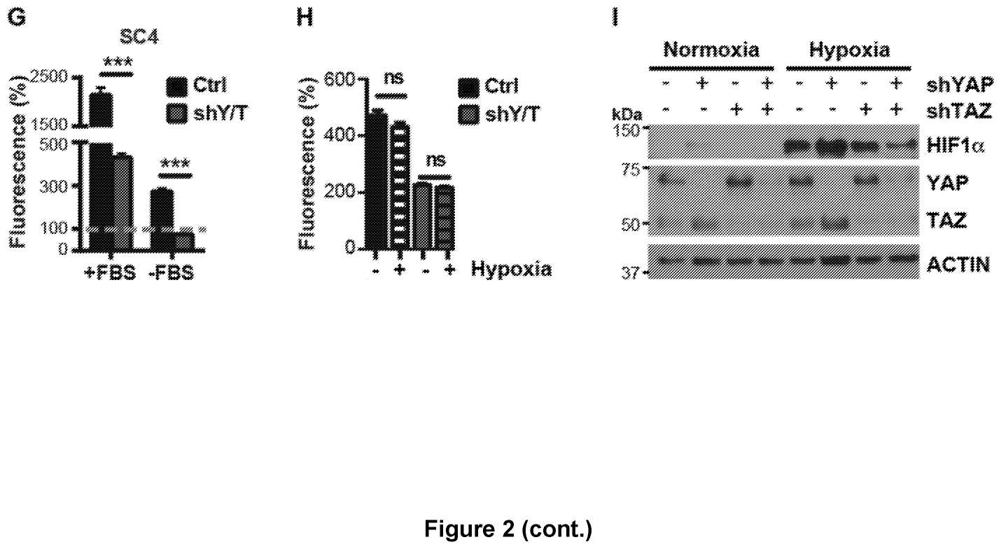

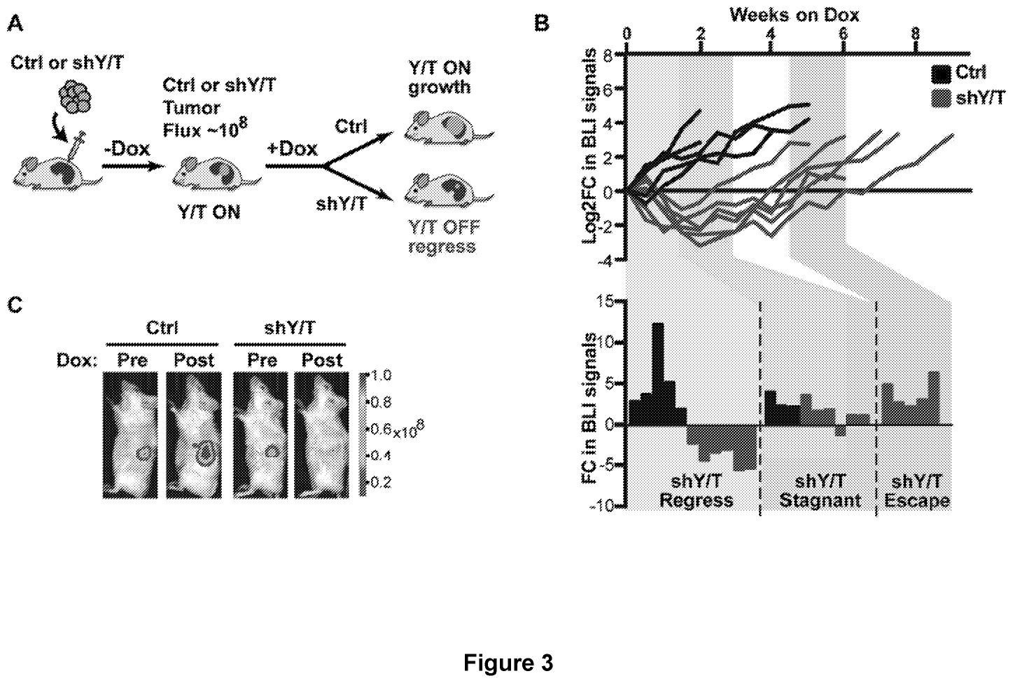

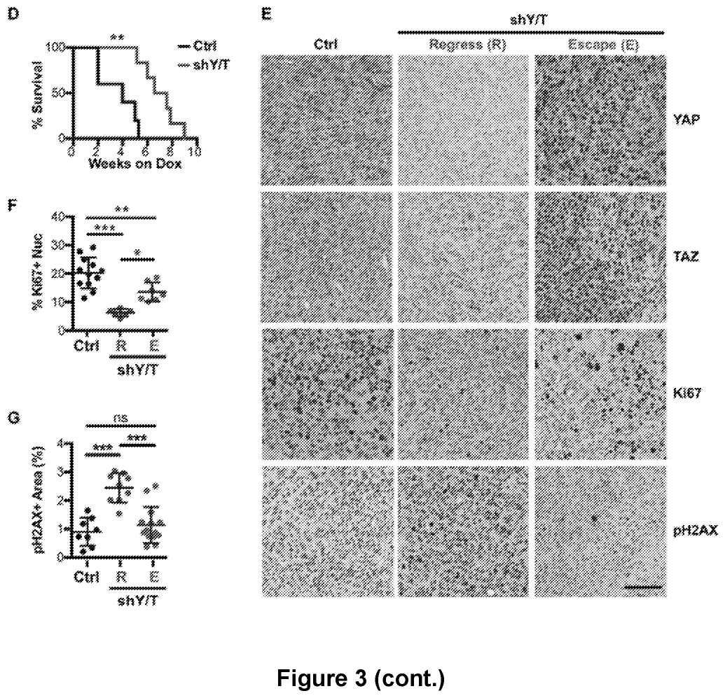

As discussed, to investigate the roles of YAP and TAZ in the maintenance of NF2-deficient tumors, Dox-inducible shRNAs against YAP and TAZ (shY/T) or a vector control (Ctrl) were stably expressed in SN12C RCC cells, which contain homozygous truncating NF2 mutations (FIG. 2A) [34]. Luciferase-labeled Ctrl and shY/T SN12C cell lines were injected orthotopically into the renal capsule of severe combined immunodeficiency (SCID)-Beige mice, and tumor growth was monitored via bioluminescent imaging (BLI) (FIG. 3A). Once a tumor signal reached a BLI flux in the magnitude of 108 photons/second corresponding to approximately 100 mm³ in tumor size (FIG. 2B), the tumor-bearing mouse was switched to a Dox-containing diet (FIG. 3A). Dox-induced YAP/TAZ depletion led to rapid reduction in BLI signals, which remained stagnant for an additional 3 weeks before starting to increase again (FIGS. 3B and 3C). In contrast, BLI signals from Ctrl tumors continued to rise steadily following Dox treatment and succumbed to the tumor burden at a significantly faster rate than shY/T tumors (FIGS. 3B-3D). Importantly, YAP/TAZ depletion did not directly alter luciferase activity, and final BLI measurements obtained immediately prior to dissection strongly correlated with the actual sizes of the resected tumors (FIGS. 2B and 2C), confirming that BLI signal changes accurately reflected the changes in tumor size in our mouse model.

Immunohistochemistry (IHC) analysis showed that compared to Ctrl tumors, the expression levels of YAP and TAZ were significantly decreased in shY/T tumors harvested during the tumor regression (R) period, which correlated with a dramatic reduction in Ki67-positive cells and a significant increase in pH2AX, a marker of DNA damage (FIGS. 3B and 3E-3G). In contrast, IHC analysis of shY/T tumors collected at later time points, when tumors started to regrow, showed restoration of YAP and/or TAZ expression throughout the tumor despite the continued Dox treatment, indicating escape (E) from the effects of the YAP/TAZ shRNAs (FIGS. 3B and 3E). Interestingly, some regions of shY/T E tumors regained either YAP or TAZ expression (FIG. 2D), suggesting that re-expression of either protein is sufficient to rescue tumor growth, as indicated by the increased percentage of Ki67+ cells and reduced numbers of pH2AX+ cells compared to shY/T tumors harvested during the R period (FIGS. 3E-3G).

Together, these results demonstrate that YAP and TAZ are required for the maintenance of NF2-deficient kidney tumors.

### YAP/TAZ Depletion Causes Defects in Glucose Usage and Increases the Reliance on Glutamine for Survival

As both Ctrl and shY/T cells express red fluorescent protein (RFP), their in vitro proliferation and survival under different conditions were tracked by comparing the percentage changes in RFP fluorescent signals over time. In contrast to the dramatic tumor R induced by YAP/TAZ depletion in vivo (FIGS. 3B and 3C), in vitro depletions of YAP/TAZ are largely cytostatic in SN12C and another Nf2-deficient SC4 murine schwannoma cell line under the standard cell culture conditions, even in the absence of serum (FIGS. 2E-2G). This apparent difference between in vitro and in vivo led to a hypothesis that in conjunction with YAP/TAZ loss, additional environmental or nutrient stress experienced by tumor cells in vivo but not under standard in vitro culture conditions might be necessary to trigger cell death in NF2-null tumor cells.

Because of a defective vasculature, tumor cells often experience hypoxia and nutrient deprivation in vivo. It was first investigated whether hypoxia could increase the dependency of SN12C cells on YAP/TAZ for growth and survival by growing Dox-treated shY/T and Ctrl cells under normoxic and hypoxic conditions. Unexpectedly, it was found that lowering oxygen levels did not significantly affect the proliferation rates or survival of either shY/T or Ctrl SN12C cells, even though it did cause the anticipated increase in HIF1a protein levels in vitro (FIGS. 2H and 2I), thus excluding hypoxia as a major contributing factor to YAP/TAZ-depletion-induced R of NF2-mutant tumors.

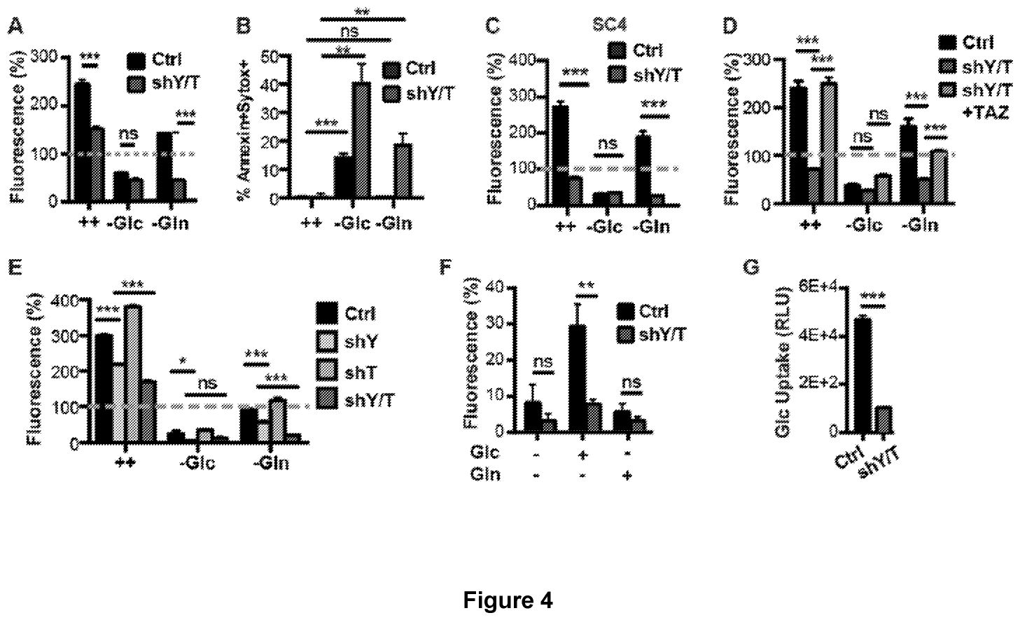

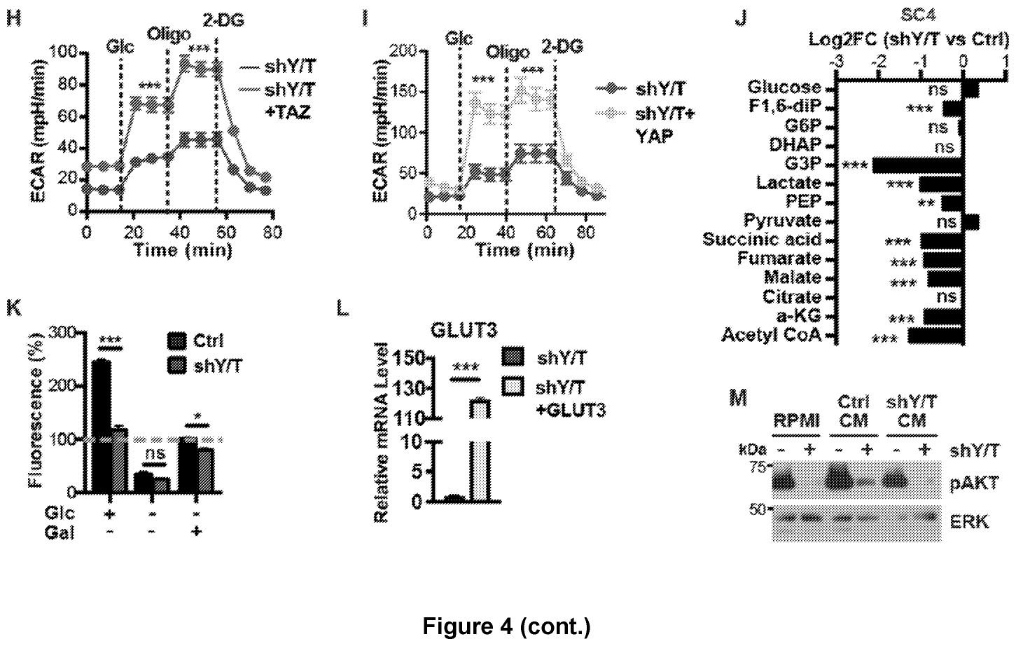

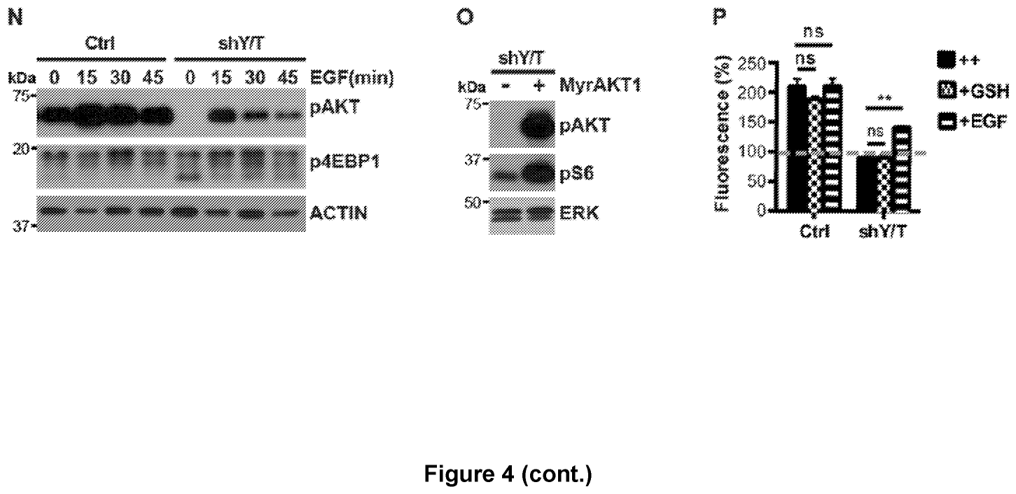

Beside hypoxia, tumor cells are often nutrient stressed in vivo because of heightened demands and limited availability. It was therefore considered whether the drastically different nutrient availabilities between the in vivo and in vitro models could contribute to the divergent effects of YAP/TAZ depletion on cell survival in vivo and in vitro. Given that glucose (Glc) and glutamine (Gln) are two major suppliers of carbon and energy for tumor cells, a test was performed to determine how Ctrl and shY/T cells respond to deprivation of Glc or Gln. Highlighting its importance in cell metabolism, complete Glc withdrawal induced massive cell death as indicated by the drops in RFP fluorescence in both Ctrl and shY/T SN12C and SC4 cells, compared to the start of treatment (FIGS. 4A-4C). In contrast, complete withdrawal of Gln caused only a minor decrease in proliferation and no significant cell death in Ctrl cells but extensive cell death in shY/T cells (FIGS. 4A-4C), which could be rescued by restoring TAZ expression (FIG. 4D). Even though knockdown of TAZ had little effects on its own across all conditions, it enhanced the growth inhibition in nutrient-replete conditions and cell death in Gln-deprived conditions caused by YAP knockdown (FIG. 4E), suggesting YAP and TAZ function redundantly in promoting the growth and survival of NF2-mutant cells.

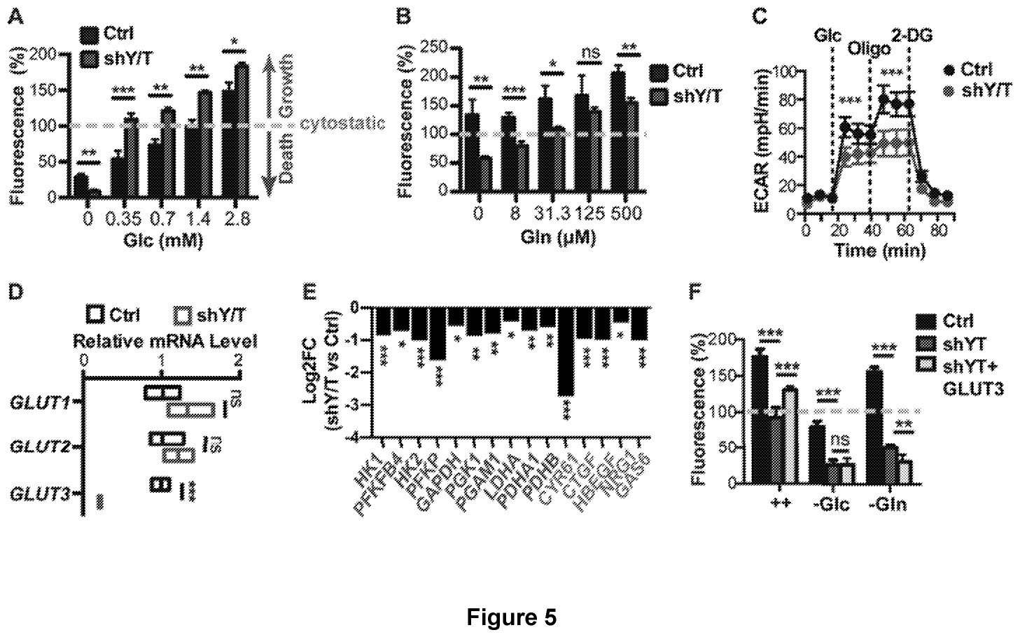

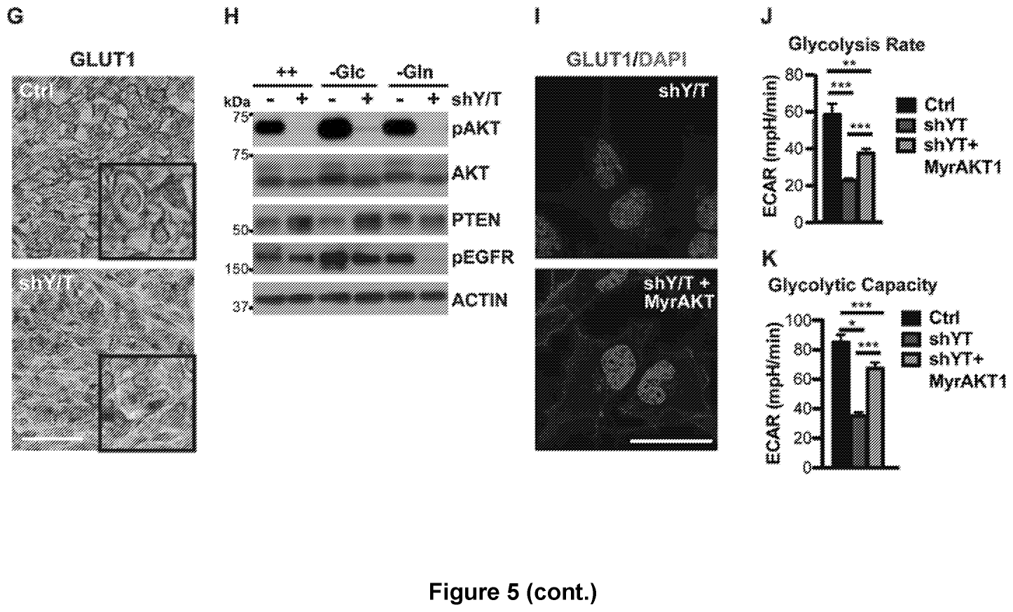

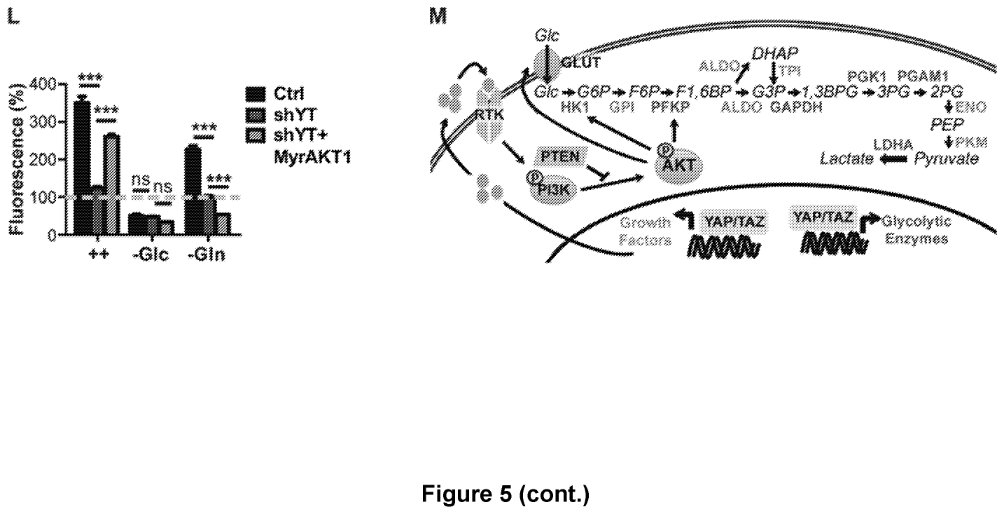

To more closely mimic the physiological conditions in tumors, Glc and Gln titration experiments were performed, which showed that Ctrl cells were generally more sensitive to reduced levels of Glc, whereas shY/T cells were more sensitive to Gln reduction (FIGS. 5A and 5B). To further assess the relative dependence of Ctrl and shY/T cells on Glc or Gln, Ctrl and shY/T cells were subjected to treatment with a media salt base supplemented with either Glc or Gln as the sole nutrient source. While the presence of Glc alone allowed a significant percentage of Ctrl cells to survive, it failed to do so in shY/T cells (FIG. 4F), again pointing to a diminished capacity for shY/T cells in utilizing Glc. On the other hand, despite the increased dependency of shY/T cells on Gln (FIG. 5B), Gln alone was not able to rescue the survival of either Ctrl or shY/T cells (FIG. 4F), implying the requirement of additional nutrients besides Gln for maintaining the survival of Ctrl and shY/T cells.

The Proliferation of NF2-Mutant Tumor Cells Is Dependent on Aerobic Glycolysis, which is Maintained by YAP/TAZ-Mediated GF-RTK-AKT Signaling and Expression of Glycolytic Enzymes

In order to meet the biosynthetic requirements of constant proliferation, tumor cells increase Glc consumption and use it primarily as a carbon source for anabolic processes rather than mitochondrial oxidative phosphorylation as in normal cells, a phenomenon known as the Warburg effect or aerobic glycolysis. In the present study, it was found that YAP/TAZ depletion dramatically reduced Glc uptake in SN12C cells (FIG. 4G). Moreover, YAP/TAZ depletion caused a marked reduction in the glycolysis rate as well as total glycolytic capacity in SN12C cells, as demonstrated by the changes in extracellular acidification rate (ECAR) following the addition of Glc and, subsequently, mitochondrial ATP synthase inhibitor oligomycin (oligo) (FIG. 5C). These glycolytic defects in shY/T SN12C cells were rescued by re-expression of either YAP or TAZ (FIGS. 4H and 4I). In further support of the roles of YAP/TAZ in promoting aerobic glycolysis, liquid chromatography-tandem mass spectrometry (LC-MS/MS) metabolic profiling of Ctrl and shY/T SC4 cells showed that YAP/TAZ depletion downregulated a number of glycolytic metabolites including G6P, G3P, PEP, and lactate (FIG. 4J).

To directly assess how aerobic glycolysis contributes to the proliferation of NF2-mutant cells, in the culture medium of SN12C cells Glc was replaced with galactose (Gal), a Glc isomer that is processed through glycolysis but does not yield any net glycolytic ATP [35]. While Gal substitution had very little effect on shY/T cells with already compromised glycolysis, it completely blocked the proliferation of Ctrl cells (FIG. 4K), underscoring the importance of high aerobic glycolysis in sustaining the proliferation of NF2-mutant tumor cells.

The molecular mechanisms underlying the role of YAP/TAZ in maintaining glycolysis was then investigated. It was found that YAP/TAZ depletion specifically reduced the mRNA levels of GLUT3 and HK2, as well as other glycolytic enzymes including HK1, PFKFB4, PFKP, GAPDH, PGK1, PGAM1, LDHA, PDHA1, and PDHB (FIGS. 5D and 5E). It was then tested whether GLUT3 downregulation and the resulting defects in Glc uptake were the main cause of the glycolytic defects in YAP/TAZ-depleted cells. Unexpectedly, ectopic expression of GLUT3 only minimally increased proliferation in nutrient-replete conditions and did not rescue cell death under nutrient-deprived conditions (FIGS. 5F and 4L), suggesting that additional mechanisms may mediate the regulation of glycolysis by YAP/TAZ.

AKT is a well-established master metabolic regulator that promotes glycolysis by inducing GLUT1 membrane localization and the activities of hexokinase and phosphofructokinase [36-42]. In the present study, it was found that even though the expression of GLUT1 was not affected by YAP/TAZ silencing, it became predominantly localized to the cytoplasm in shY/T tumors in contrast to the typical membrane localization displayed in Ctrl tumors (FIGS. 5D and 5G). To test whether YAP/TAZ could act through AKT to promote GLUT1 membrane translocation and glycolysis, the levels of pAKT in Ctrl and shY/T SN12C cells were compared. As shown in FIG. 5H, YAP/TAZ silencing caused a substantial decrease in AKT phosphorylation, which correlated with reduced pEGFR and increased PTEN levels, implying that downregulation of RTK signaling as the likely cause of AKT inactivation. Microarray analysis showed that a number of growth factors (GFs), including canonical YAP/TAZ targets CYR61 and CTGF, EGF-family GFs HBEGF and NRG1, and GAS6, were downregulated in shY/T cells compared to Ctrl cells (FIG. 5E). Moreover, treatment of shY/T cells with EGF or conditioned medium (CM) collected from Ctrl cells partially rescued AKT phosphorylation (FIGS. 4M and 4N). These results suggest that reduction in GF-RTK signaling is at least partially responsible for AKT inactivation and growth arrest in shY/T cells.

To assess how downregulation of AKT signaling contributes the phenotypes induced by YAP/TAZ knockdown, a shY/T cell line stably expressing a constitutively active AKT1 (shY/T+MyrAKT1) was generated (FIG. 40). Immunofluorescence (IF) and a glycolysis stress test showed that restoration of AKT signaling rescued GLUT1 membrane localization in shY/T cells and partially reversed the suppression of glycolysis and glycolytic capacity caused by YAP/TAZ depletion (FIGS. 5I-5K). Correspondingly, reactivation of AKT signaling by either expression of MyrAKT or EGF treatment allowed shY/T cells to regain the ability to proliferate in nutrient-replete conditions (FIGS. 5L and 4P).

Together, these findings reconciled previously reported functions of YAP/TAZ in glycolysis and regulation of RTK-AKT signaling, establishing YAP/TAZ as master regulators that coordinate the expression of glycolytic enzymes and GFs and RTK-AKT signaling to promote glycolysis, thereby sustaining the proliferation of NF2-mutant tumor cells (FIG. 5M).

YAP/TAZ Depletion Increases Mitochondrial Respiration and ROS Buildup, Causing Oxidative-Stress-Induced Cell Death under Nutrient-Deprived Conditions

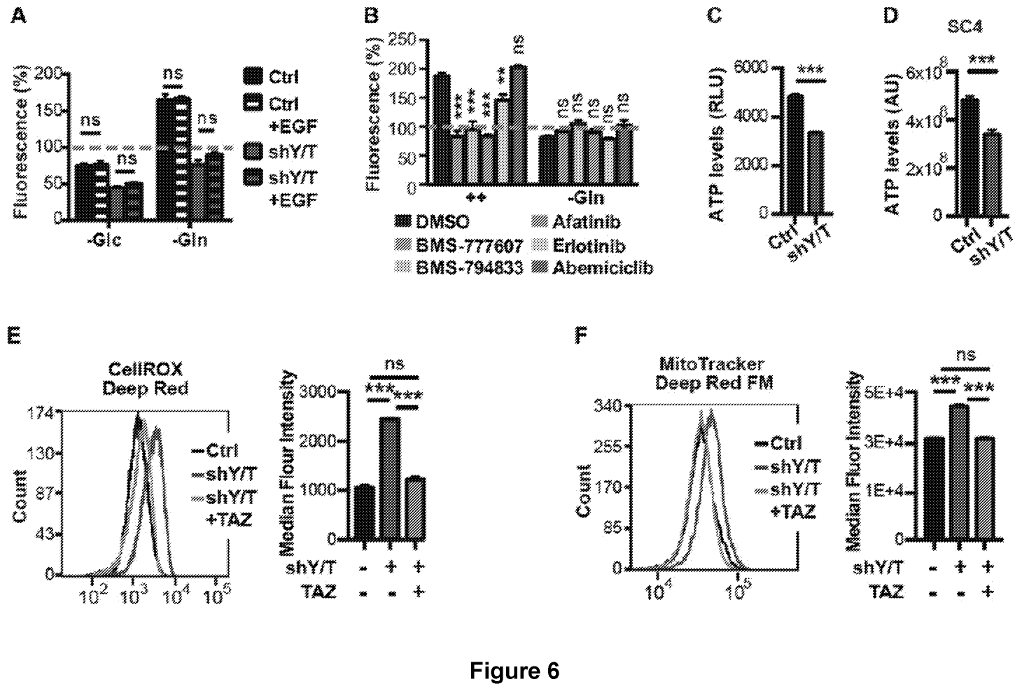

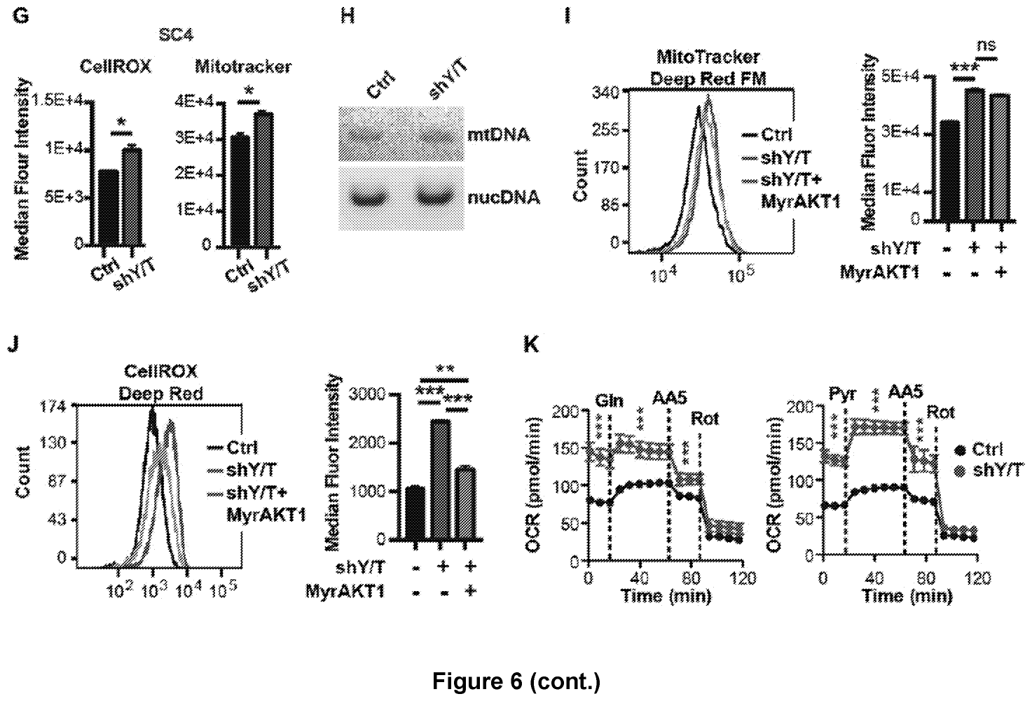

Although restoration of AKT signaling, either by expression of constitutively active AKT or treatment with EGF, rescued the shY/T cell proliferation in nutrient-replete conditions, it did not prevent cell death induced by Glc or Gln withdrawal (FIGS. 5L, 4P, and 6A). In agreement, treatment of Ctrl SN12C cells with various RTK inhibitors readily blocked cell proliferation but did not cause any cell death even when Gln was removed (FIG. 6B). These results indicate that while GF-RTK-AKT signaling is important for maintaining glycolysis-dependent proliferation, other mechanisms govern their survival.

Despite the profound downregulation in glycolysis, shY/T cells remained mostly viable in nutrient-replete conditions (FIGS. 4A-4C) and only showed a slight reduction in ATP levels relative to Ctrl cells (FIGS. 6C and 6D). Without being bound by theory, it was postulated that shY/T cells might compensate for the deficit in glycolysis by upregulating mitochondrial oxidative phosphorylation. To assess mitochondrial respiration in Ctrl and shY/T cells, we measured their basal oxygen consumption rates (OCR), as well as changes in OCR following sequential injections of oligomycin, carbonyl cyanide-4-(trifluoromethoxy)phenylhydrazone (FCCP; ATP synthesis uncoupler), and rotenone/antimycin A (Rot/AMA; complex I/III inhibitors). Corresponding to downregulation in glycolysis, both basal respiration rates as reflected by basal OCRs and mitochondrial respiratory capacity as measured by an increase in OCR induced by FCCP were significantly increased in shY/T cells compared to Ctrl cells (FIGS. 5C and 1A).

Mitochondrial respiration is the primary source of ROS in the cell. To remove excess ROS and repair oxidative damages, cells have developed an anti-oxidant network that heavily relies on Glc and Gln metabolism to generate NADH, NADPH, and GSH to maintain its reducing capacity. It was hypothesized that the increase in mitochondrial respiration induced by YAP/TAZ depletion might lead to elevated ROS production, which when compounded by reduced antioxidant capacity caused by Glc or Gln starvation, might cause oxidative-stress-induced cell death. To test this, shY/T SN12C cells were stained with fluorescent probes that specifically measure intracellular ROS levels (CellROX) and mitochondrial mass (MitoTracker) prior to (day 0) or after different days of Dox treatment. It was found that YAP/TAZ depletion induced gradual increases in both ROS levels and mitochondrial mass (FIG. 1B), both of which were reversed upon TAZ re-expression (FIGS. 6E and 6F). Similarly, YAP/TAZ depletion also increased ROS levels and mitochondrial mass in SC4 cells (FIG. 6G). The increase in mitochondrial mass was also confirmed by IF analysis with a mitochondria-specific antibody and by electron microscopy (EM) analysis (FIGS. 1C and 1D). In contrast, the total numbers of mitochondria per cell appeared to be unchanged based on analyses of EM images and the mitochondrial DNA copy numbers (FIG. 6H). Corresponding to increases in mitochondrial mass and intracellular ROS levels, shY/T cells exhibited increased expression of key subunits of all five OXPHOS complexes (FIG. 1E), elevated mitochondria-derived ROS as measured by Mitosox, and a significant rise in the ratio of MitoSox to MitoTracker staining (FIG. 1F).

The citric acid (TCA) cycle produces NADH and succinate, which serve as the substrates for OXPHOS complex I and complex II, respectively. Given the increase in mitochondrial mass and OXPHOS in shY/T cells, the steady-state levels of TCA metabolites in Ctrl and shY/T cells was assessed using LC-MS/MS analysis. Unexpectedly, with the exception of pyruvate (Pyr) and citrate, the majority of TCA intermediates were significantly downregulated following YAP/TAZ depletion (FIG. 4J). To test whether this could be caused by an increased TCA flux stemming from increased OXPHOS in YAP/TAZ-depleted cells, OXPHOS complex I and complex II activity assays were performed in Ctrl and shY/T SN12C cells. To measure complex I activity, Pyr and malate (Mal) were added to permeabilized Ctrl and shY/T cells to drive the production of the complex I substrate NADH, followed by injections of Rot, Atpenin A5 (AA5), and AMA to sequentially block complexes I, II, and III, respectively (FIGS. 1G and 1H). To determine complex II activity, Rot was added to block any complex-I-mediated respiration prior to treatment with the complex II substrate succinate, followed by injections of AA5 and AMA to inactivate complexes II and III, respectively (FIGS. 1G and 1H). These experiments showed that YAP/TAZ depletion dramatically boosted the activities of both complex I and complex II, suggesting that increased OXPHOS and TCA flux likely contributes to the downregulation of TCA intermediates in shY/T cells.

To assess whether decreased glycolysis caused a compensatory upregulation of mitochondrial respiration in YAP/TAZ knockdown cells, the mitochondrial activities and ROS levels in shY/T cells reconstituted with vector Ctrl or Myr-AKT were compared. Although expression of Myr-AKT largely rescued glycolysis and proliferation in shY/T cells, it did not reduce mitochondrial mass, only partially reversed the increase in intracellular ROS levels, and failed to prevent cell death caused by Gln withdrawal in shY/T cells (FIGS. 5J, 5L, 6I, and 6J).

Next, Pyr or Gln were used to directly stimulate TCA cycle and OXPHOS in Ctrl and shY/T cells, bypassing glycolysis. As shown in FIG. 6K, both Pyr and Gln increased mitochondrial respiration to a significantly higher extent in YAP/TAZ-depleted cells, suggesting that YAP/TAZ suppress mitochondrial respiratory capacity and ROS production independent of their regulation of glycolysis.

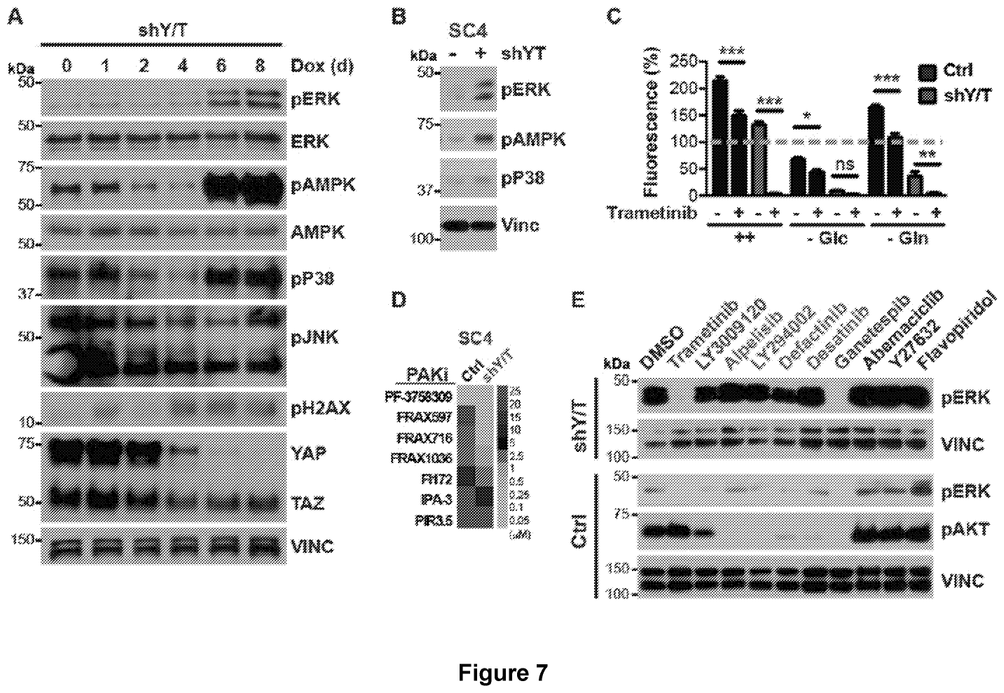

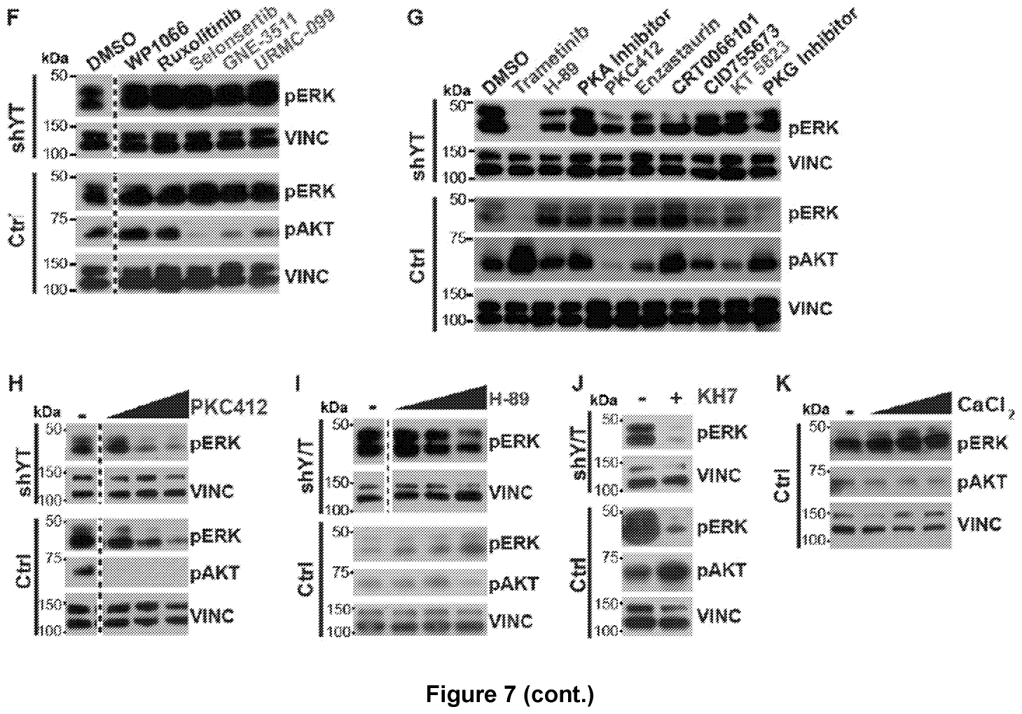

Indicative of increased oxidative stress, H2AX was activated both in vitro and in vivo following YAP/TAZ knockdown (FIGS. 3E, 3G, and 7A), which correlated with significant increases in the NAD+/NADH, NADP+/NADPH, and GSSG/GSH ratios (FIG. 1I). To determine whether oxidative stress was the cause of cell death in Glc- or Gln-deprived shY/T cells, ROS levels in Ctrl and shY/T SN12C cells in the presence or absence of Glc or Gln were measured. As expected, withdrawal of Glc or Gln raised ROS levels in both Ctrl and shY/T cells (FIG. 1J). However, the levels of ROS were substantially higher in shY/T cells than Ctrl cells across all conditions (FIG. 1J). Ctrl and shY/T cells were then treated with GSH under the different nutrient conditions. While GSH treatment did not affect the proliferation of either Ctrl or shY/T cells under nutrient-replete conditions (FIG. 4P), it significantly inhibited both Glc-and Gln-starvation-induced cell death in shY/T cells but had no effect on Ctrl cells (FIG. 1K), confirming that additional oxidative stress induced by nutrient deprivation triggers cell death in shY/T cells.

Together, the findings expose a function for YAP/TAZ in limiting mitochondrial respiratory capacity and ROS production, which is necessary for maintaining redox balance and survival of NF2-mutant tumor cells under nutrient-deprived conditions.

NF2-Mutant Tumor Cells Adapt to YAP/TAZ Depletion through Activation of a Noncanonical cAMP-PKA/EPACRAF-MEK-ERK Signaling Cascade

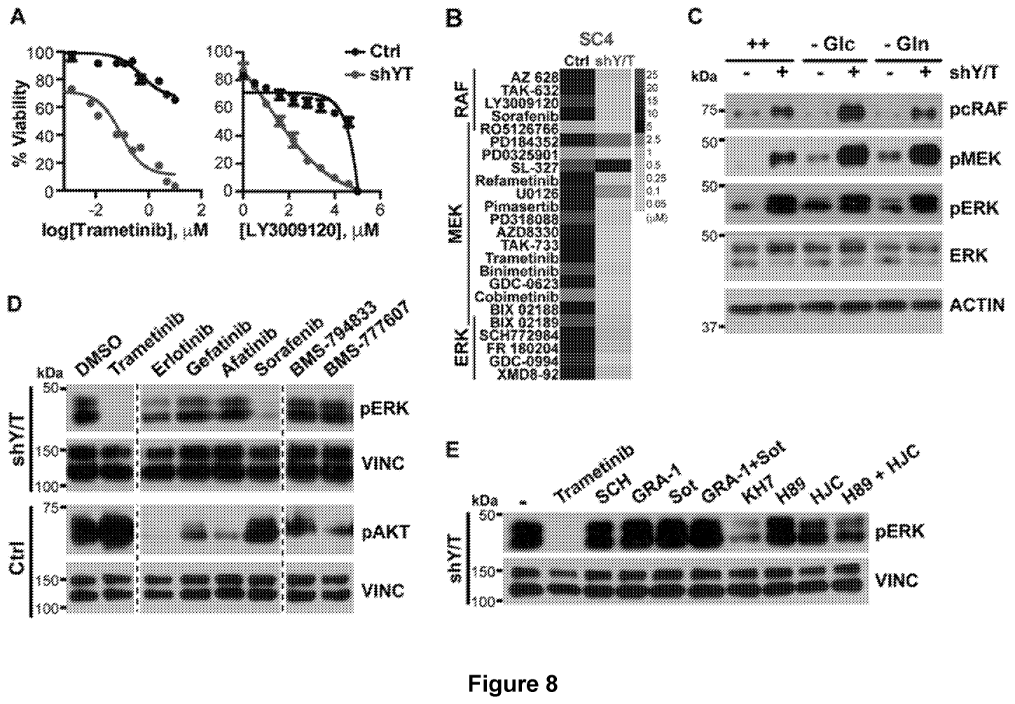

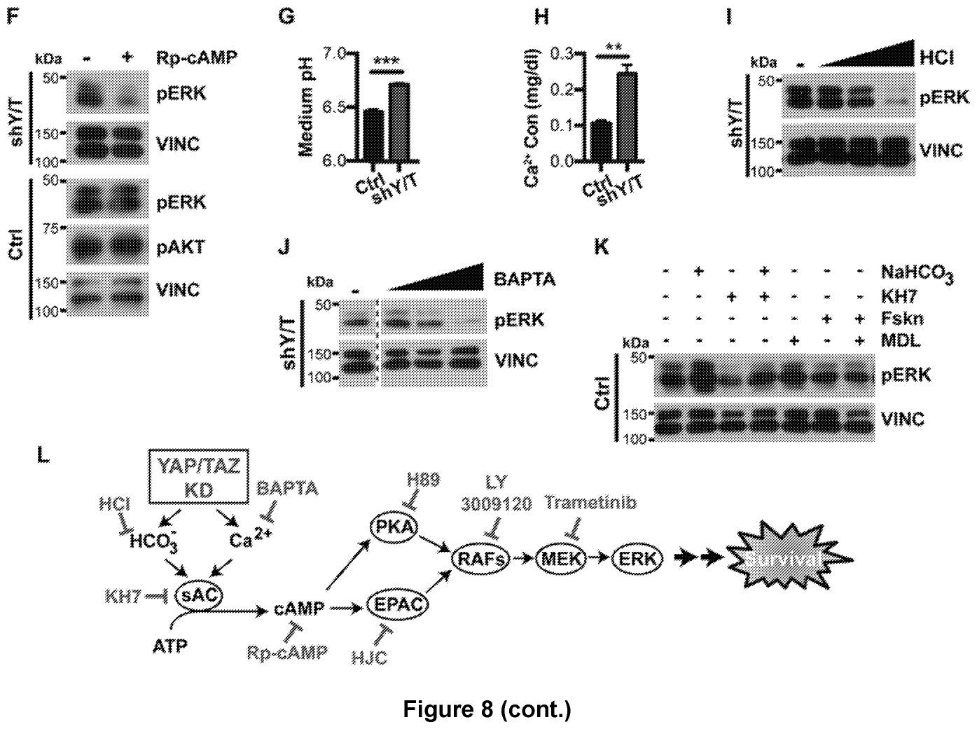

The in vitro and in vivo data indicate that although NF2-mutant tumor cells require YAP/TAZ for proliferation, they are capable of surviving YAP/TAZ loss and the resulting rise in ROS levels and oxidative stress when both Glc and Gln are readily available. This led to an investigation of whether NF2-mutant tumor cells could activate certain stress-response pathways to counter the redox imbalance caused by YAP/TAZ loss. Indeed, western blot analysis showed that multiple stress-response pathways including ERK, AMPK, and p38 were activated in response to YAP/TAZ knockdown (FIGS. 7A and 7B). To test how these pathways contribute to the survival of YAP/TAZ-depleted cells, Ctrl and shY/T SN12C and SC4 cells were treated with selective inhibitors targeting each of these pathways. Out of the inhibitors screened, the RAF/MEK/ERK inhibitors exhibited the strongest selective killing of YAP/TAZ-depleted cells compared to Ctrl cells (FIGS. 8A, 8B, and 7C). Consistent with this finding, western blot analysis showed a robust increase in c-RAF, MEK, and ERK phosphorylation levels in shY/T cells relative to Ctrl cells across all nutrient conditions (FIG. 8C). Furthermore, it was found that multiple inhibitors against the group I PAKs, which directly phosphorylate and are required for the activation of RAF and MEK [43-46], also showed some degrees of selective killing of shY/T cells compared to Ctrl cells (FIG. 7D). These results demonstrate that activation of the RAF-MEK-ERK pathway is necessary for the survival of YAP/TAZ-depleted NF2-mutant cells.

The RAF-MEK-ERK pathway is canonically activated by RTK-RAS signaling. However, treatment with various RTK inhibitors at concentrations that caused robust inhibition of pAKT in Ctrl cells failed to reduce pERK levels in shY/T cells (FIGS. 8D and 7E). In contrast, the MEK inhibitor trametinib and pan-RAF inhibitors LY3009120 and Sorafenib inhibited pERK levels in shY/T cells but did not reduce pAKT levels in Ctrl cells (FIGS. 8D and 7E). These findings indicate a noncanonical, alternative mechanism likely to be responsible for RAF-MEK-ERK activation in shY/T cells. To identify the pathway(s) responsible, an inhibitor screen was conducted against a wide array of kinase and non-kinase targets including FAK/SRC, STATS, CDKs, MLKs, PKA, PKC, PKD, PKG, and additional MAPKs (table in FIG. 14). Of all the inhibitors screened, only H-89, a PKA inhibitor, specifically inhibited ERK phosphorylation in shY/T cells without also blocking pAKT in Ctrl cells (FIGS. 7F-7I).

PKA is activated by the second messenger cyclic AMP (cAMP), which also directly binds to and activates EPAC. PKA and EPAC were previously reported to function in parallel to activate the RAF-MEK-ERK pathway independently of RTK [47-49]. It was found that PKA inhibitor H-89 synergizes with EPAC inhibitor HJC in reducing pERK levels in shY/T cells (FIG. 8E). Similarly, an analog and competitive inhibitor of cAMP, Rp-cAMP, also specifically inhibited ERK phosphorylation in shY/T cells without affecting pAKT in Ctrl cells (FIG. 8F). cAMP is synthesized by either transmembrane adenylyl cyclase (tmAC) or soluble adenylyl cyclase (sAC). tmAC is activated by G-protein-coupled receptor (GPCR) signaling, whereas sAC, which is dispersed throughout the cytoplasm, is stimulated in response to elevated bicarbonate (HCO₃⁻) and calcium [50, 51]. To determine which of these two mechanisms were responsible for activation of cAMP-PKA/EPAC signaling upon YAP/TAZ depletion, shY/T cells were treated with GPCR inhibitors (SCH 202676, GRA-1, or Sotalol) or an sAC inhibitor (KH7). While none of the GPCR inhibitors reduced ERK phosphorylation, the sAC inhibitor KH7 drastically inhibited pERK levels in shY/T cells but not pAKT in Ctrl cells (FIGS. 8E and 7J). To identify the signal that led to sAC activation, the pH and calcium levels were measured in Ctrl and shY/T cells. Supernatant collected from shY/T cells exhibited a significantly higher pH than Ctrl cells (FIG. 8G). In addition, intracellular calcium concentration was markedly increased in shY/T cells compared to Ctrl cells (FIG. 8H). To test whether the increases in pH and calcium levels were indeed responsible for the activation of ERK in shY/T cells, these cells were treated with increasing concentrations of HCl or calcium chelator BAPTA, both of which reduced pERK levels in a dose-dependent manner (FIGS. 8I-8J). Conversely, Ctrl cells treated with increasing concentrations of calcium displayed increased pERK levels (FIG. 7K). Bicarbonate treatment also raised pERK levels in Ctrl cells, which was blocked by KH7 (FIG. 8K). In contrast, treatment with forskolin (tmAC activator) or MDL 12330A (tmAC inhibitor) alone or in combination had no effect on ERK phosphorylation (FIG. 8K).

Together, these results illustrate that NF2-deficient tumor cells survive YAP/TAZ depletion through noncanonical activation of the RAF-MEK-ERK pathway, which is mediated by a signaling cascade involving the elevation of intracellular pH and calcium levels and the subsequent induction of sAC and cAMP-PKA/EPAC signaling (FIG. 8L).

Elevated Lysosomal Activity Is Responsible for ERK Activation and Survival of NF2-Mutant Tumor Cells upon YAP/TAZ Depletion

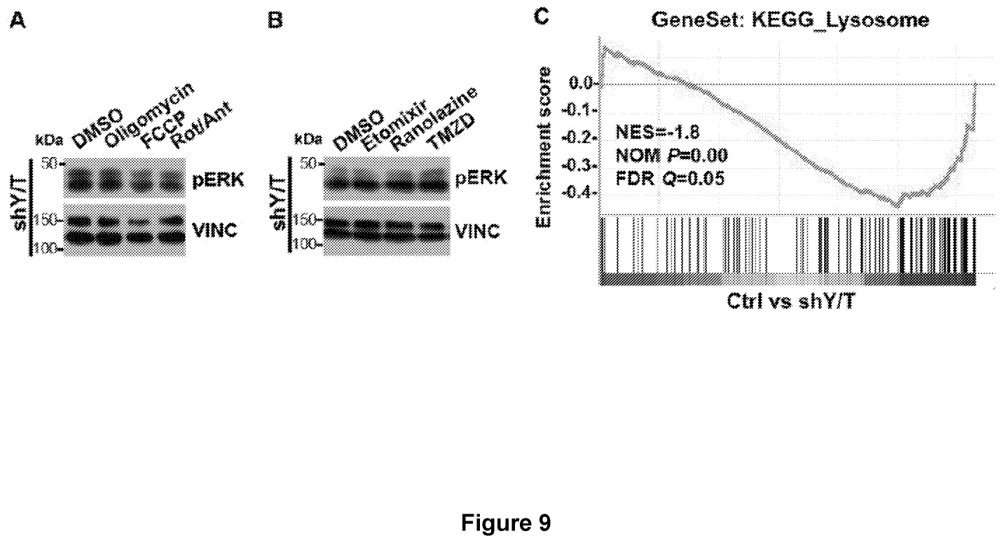

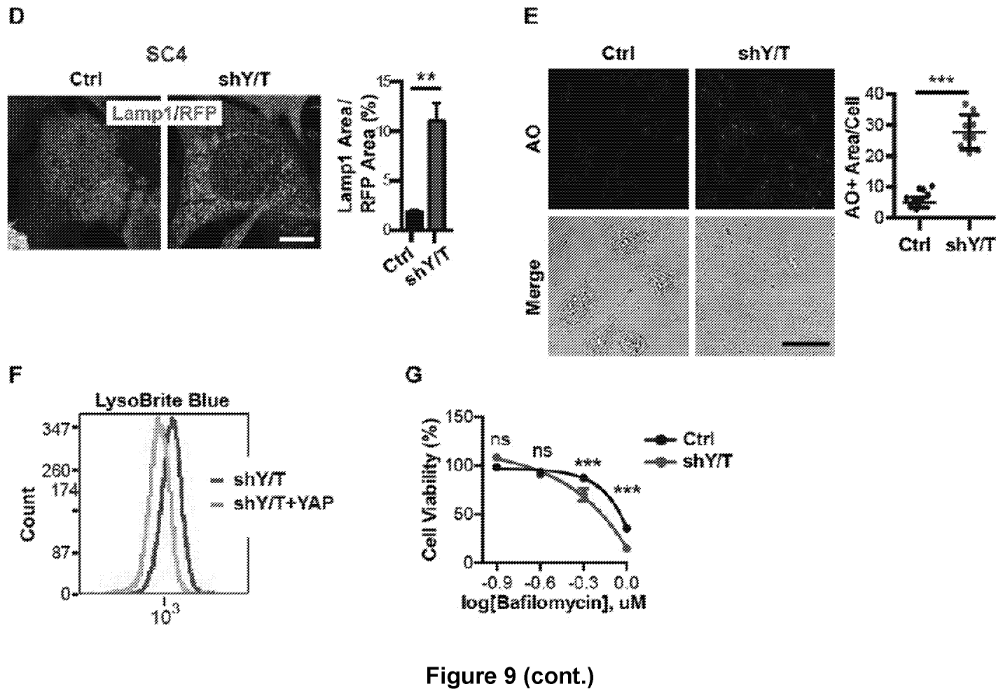

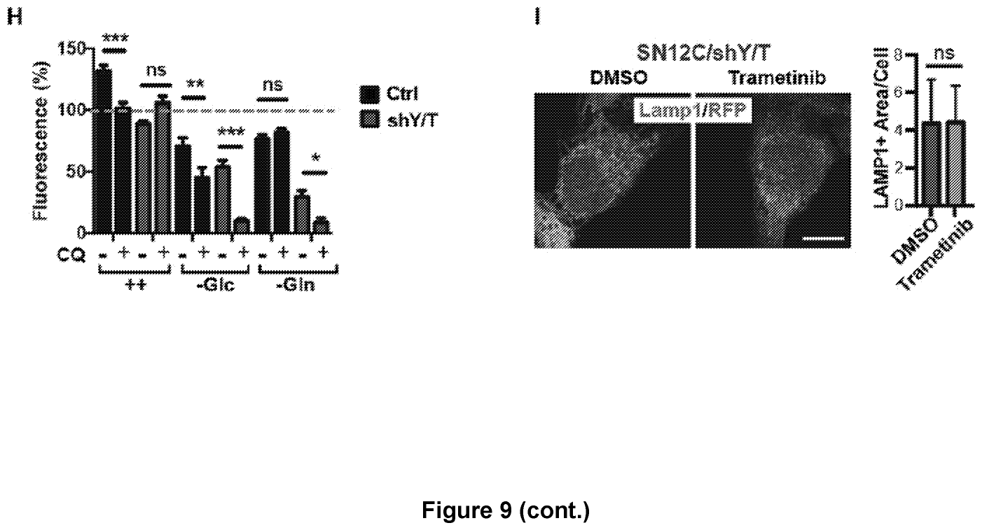

Next, identification was sought of the source(s) of elevated intracellular pH and calcium levels that caused the noncanonical activation of ERK in shY/T cells. Mitochondria are major calcium storage sites, and generators of CO₂-derived bicarbonate through mitochondrial carbonic anhydrase [52-54]. Therefore, it was first investigated whether the dramatic increase in mitochondrial capacity and respiratory activity induced by YAP/TAZ silencing could be the cause of noncanonical ERK activation. However, it was found that multiple mitochondrial inhibitors targeting the ATP-synthase, ETC complexes, carbonic anhydrase, or β-oxidation had no effect on ERK phosphorylation in shY/T cells (FIGS. 9A and 9B), suggesting that elevated mitochondrial capacity and respiratory activity were not the cause of the pH and calcium increase following YAP/TAZ loss.

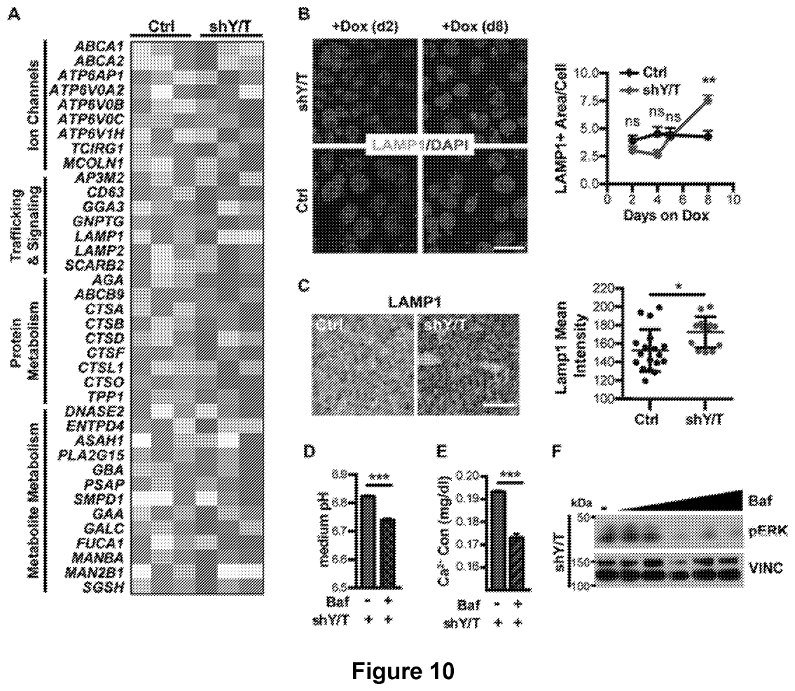

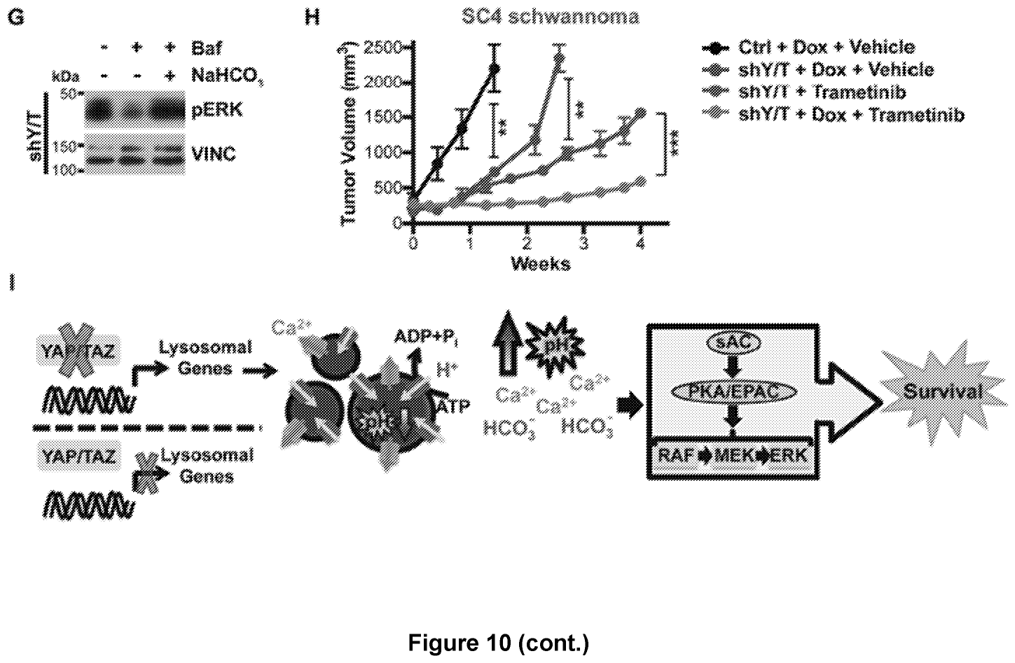

Lysosomes are small vesicles characterized by a highly acidic (pH 4.5-5.0) interior containing numerous hydrolytic enzymes, which function as cellular trafficking stations to facilitate the breakdown and recycling of a wide range of both endogenous and exogenous cargo including macromolecules, certain pathogens, and damaged organelles. Gene set enrichment analysis (GSEA) showed strong enrichment of the KEGG_Lysosome gene set among genes upregulated in response to YAP/TAZ silencing (FIGS. 10A and 9C). In line with this finding, staining of Ctrl and shY/T cells with a LAMP1 antibody or acridine orange (AO; marker of acidic vesicles) showed that YAP/TAZ knockdown caused a marked increase in the numbers of lysosomes, which was rescued by re-expression of YAP (FIGS. 10B and 9D-9F). In agreement with these in vitro findings, IHC analysis confirmed that LAMP1 levels were also significantly elevated in shY/T tumors compared to Ctrl tumors (FIG. 10C).

Lysosome biogenesis and autophagy play key roles in salvaging nutrients and degrading damaged macromolecules and organelles to promote cell survival under stress conditions [55, 56]. On the other hand, in the presence of severe and irreversible damages, lysosomal membrane permeabilization and the consequent leakage of the lysosomal content into the cytosol could lead to so-called “lysosomal cell death” [57]. Treatment of Ctrl and shY/T cells with two different lysosome inhibitors (bafilomycin and chloroquine) under different nutrient conditions showed that YAP/TAZ depletion increased the sensitivity of NF2-mutant cells to lysosomal inhibition, especially under nutrient-deprived conditions (FIGS. 9G and 9H). These results imply that increased lysosome biogenesis plays a largely pro-survival role in YAP/TAZ-depleted cells. RAF-MEK-ERK signaling was previously reported to regulate lysosomal biogenesis and autophagy [58, 59]. However, a significant change in LAMP1 staining following trametinib treatment in shY/T cells was not detected (FIG. 9I). On the other hand, treatment of shY/T cells with bafilomycin, which blocks lysosomal acidification by inhibiting the vacuolar-type H⁺ ATPase (v-ATPase), significantly reduced pH and calcium levels (FIGS. 10D and 10E) and inhibited ERK phosphorylation in a dose-dependent manner (FIG. 10F). Notably, bafilomycin-mediated ERK inhibition was rescued by the addition of exogenous HCO₃⁻ (FIG. 10G), confirming the importance of lysosome-mediated intracellular pH regulation in modulating ERK activity.

Finally, the efficacy of dual inhibition of YAP/TAZ and MAPK signaling in controlling the growth of SC4 schwannomas in vivo was tested. SC4 cells carrying Dox-inducible shY/T or vector Ctrl were injected subcutaneously into the flanks of SCID-Beige mice. Once the tumors reached approximately 100 mm³, mice bearing shY/T tumors were randomly assigned to one of the following three treatment arms: Dox+vehicle, trametinib, or Dox+trametinib, whereas mice bearing Ctrl tumors were treated with Dox+vehicle. While either YAP/TAZ depletion or trametinib treatment alone significantly delayed tumor growth compared to Ctrl tumors, simultaneous YAP/TAZ knockdown and Mek inhibition halted tumor growth for several weeks (FIG. 10H).

Taken together, the data demonstrate that NF2-mutant tumor cells compensate for YAP/TAZ loss by expanding their lysosomal capacity, which raises the cytosolic pH and calcium concentration, leading to the activation of RAF-MEK-ERK signaling, which represents a vulnerability that could be combined with YAP/TAZ inhibition to achieve more durable Ctrl of NF2-mutant tumors (FIG. 10I).

Correlation of a YAP/TAZ Transcription Signature with the Expression of Glycolysis, OXPHOS, and Lysosomal Genes in Human RCC Tumors

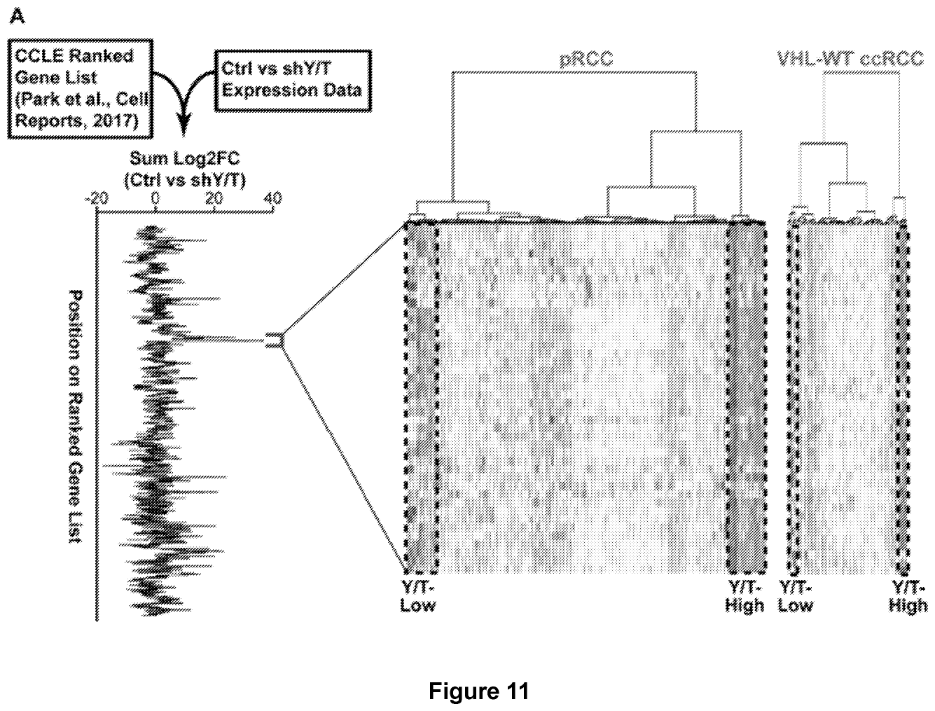

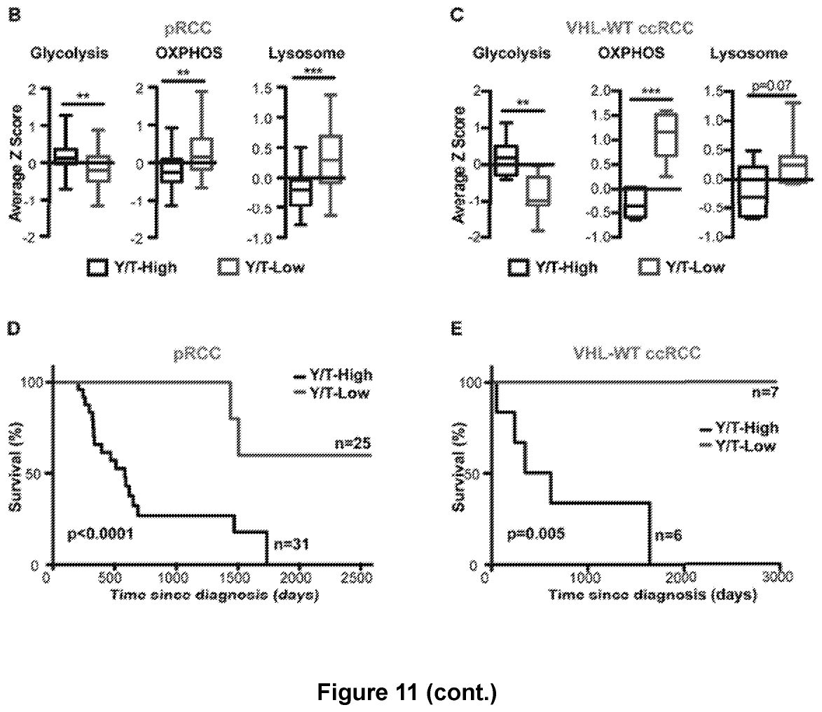

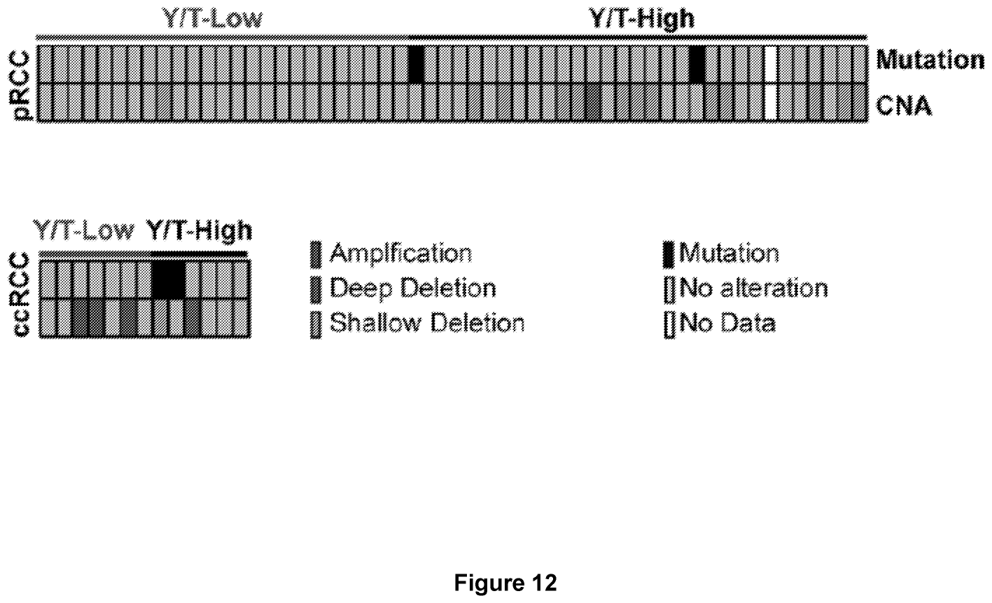

To assess the clinical relevance of the findings, a high-confidence YAP/TAZ signature was generated by filtering genes downregulated by YAP/TAZ knockdown from the microarray analysis of SN12C cells against a recently published gene list ranked based on gene expression pattern similarities across 1,037 cancer cell lines from the Cancer Cell Line Encyclopedia (CCLE) (FIG. 11A) [32]. Using this 44-gene YAP/TAZ signature, unsupervised clustering was performed of all publicly available pRCC and VHL-WT ccRCC expression datasets from The Cancer Genome Atlas (TCGA) [60, 61] and selected the tumor clusters expressing the highest (Y/T-High) and lowest (Y/T-Low) YAP/TAZ gene signature from each dataset for further analyses (FIG. 11A; tables in FIGS. 13 and 15). Importantly, pRCC and ccRCC tumors with either NF2 mutations or copy number loss were enriched in Y/T-High groups compared to Y/T-Low groups, further validating our YAP/TAZ signature (FIG. 12).

In agreement with the findings in NF2-mutant cells, primary RCC tumors from the Y/T-High groups displayed elevated expression of glycolysis genes and correspondingly decreased expression of OXPHOS and lysosomal genes compared to the tumors from the Y/T-Low groups in both pRCC and VHL-WT ccRCC datasets (FIGS. 11B and 11C; table in FIG. 13). Moreover, patients from the Y/T-High groups had poorer survival rates compared to their Y/T-Low counterparts in both the pRCC (p<0.0001) and VHL-WT ccRCC (p=0.005) (FIGS. 11D and 11E). Taken together, these results suggest YAP/TAZ activities may play important roles in determining the metabolic states of RCC tumors beyond NF2 mutations.

The foregoing description is given for clearness of understanding only, and no unnecessary limitations should be understood therefrom, as modifications within the scope of the invention may be apparent to those having ordinary skill in the art.

Detailed embodiments of the present methods and magnetic devices are disclosed herein; however, it is to be understood that the disclosed embodiments are merely illustrative and that the methods and magnetic devices may be embodied in various forms. In addition, each of the examples given in connection with the various embodiments of the systems and methods are intended to be illustrative, and not restrictive.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

### REFERENCES

[1] Petrilli, A. M., and Fernańdez-Valle, C. (2016). Role of Merlin/NF2 inactivation in tumor biology. Oncogene 35, 537-548.
[2] Flaiz, C., Ammoun, S., Biebl, A., and Hanemann, C. O. (2009). Altered adhesive structures and their relation to RhoGTPase activation in merlin-deficient schwannoma. Brain Pathol. 19, 27-38.
[3] Houshmandi, S. S., Emnett, R. J., Giovannini, M., and Gutmann, D. H. (2009). The neurofibromatosis 2 protein, merlin, regulates glial cell growth in an ErbB2- and Src-dependent manner. Mol. Cell. Biol. 29, 1472-1486.
[4] Kaempchen, K., Mielke, K., Utermark, T., Langmesser, S., and Hanemann, C. O. (2003). Upregulation of the Rac1/JNK signaling pathway in primary human schwannoma cells. Hum. Mol. Genet. 12, 1211-1221.
[5] Li, N., Batzer, A., Daly, R., Yajnik, V., Skolnik, E., Chardin, P., Bar-Sagi, D., Margolis, B., and Schlessinger, J. (1993). Guanine-nucleotide-releasing factor hSos1 binds to Grb2 and links receptor tyrosine kinases to Ras signalling. Nature 363, 85-88.
[6] Morrison, H., Sperka, T., Manent, J., Giovannini, M., Ponta, H., and Herrlich, P. (2007). Merlin/neurofibromatosis type 2 suppresses growth by inhibiting the activation of Ras and Rac. Cancer Res. 67, 520-527.
[7] Nakai, Y., Zheng, Y., MacCollin, M., and Ratner, N. (2006). Temporal control of Rac in Schwann cell-axon interaction is disrupted in NF2-mutant schwannoma cells. J. Neurosci. 26, 3390-3395.
[8] Rong, R., Tang, X., Gutmann, D. H., and Ye, K. (2004). Neurofibromatosis 2 (NF2) tumor suppressor merlin inhibits phosphatidylinositol 3-kinase through binding to PIKE-L. Proc. Natl. Acad. Sci. USA 101, 18200-18205.
[9] Shaw, R. J., Paez, J. G., Curto, M., Yaktine, A., Pruitt, W. M., Saotome, I., O'Bryan, J. P., Gupta, V., Ratner, N., Der, C. J., et al. (2001). The Nf2 tumor suppressor, merlin, functions in Rac-dependent signaling. Dev. Cell 1, 63-72.
[10] Yi, C., Troutman, S., Fera, D., Stemmer-rachamimov, A., Avila, L., Christian, N., Persson, N. L., Shimono, A., David, W., Marmorstein, R., et al. (2011). A tight junction-associated merlin-Angiomotin complex mediates merlin's regulation of mitogenic signaling and tumor suppressive functions. Cancer Cell 19, 527-540.
[11] Blakeley, J. O., Evans, D. G., Adler, J., Brackmann, D., Chen, R., Ferner, R. E., Hanemann, C. O., Harris, G., Huson, S. M., Jacob, A., et al. (2012). Consensus recommendations for current treatments and accelerating clinical trials for patients with neurofibromatosis type 2. Am. J. Med. Genet. A158A, 24-41.
[12] Goutagny, S., Raymond, E., Esposito-Farese, M., Trunet, S., Mawrin, C., Bernardeschi, D., Larroque, B., Sterkers, O., Giovannini, M., and Kalamarides, M. (2015). Phase II study of mTORC1 inhibition by everolimus in neurofibromatosis type 2 patients with growing vestibular schwannomas. J. Neurooncol. 122, 313-320.
[13] Huang, J., Wu, S., Barrera, J., Matthews, K., and Pan, D. (2005). The hippo signaling pathway coordinately regulates cell proliferation and apoptosis by inactivating Yorkie, The Drosophila homolog of YAP. Cell 122, 421-434.
[14] Zhao, B., Wei, X., Li, W., Udan, R. S., Yang, Q., Kim, J., Xie, J., Ikenoue, T., Yu, J., Li, L., et al. (2007). Inactivation of YAP oncoprotein by the Hippo pathway is involved in cell contact inhibition and tissue growth control. Genes Dev. 21, 2747-2761.
[15] Callus, B. A., Verhagen, A. M., and Vaux, D. L. (2006). Association of mammalian sterile twenty kinases, Mst1 and Mst2, with hSalvador via C-terminal coiled coil domains, leads to its stabilization and phosphorylation. FEBS J. 273, 4264-4276.
[16] Plouffe, S. W., Meng, Z., Lin, K. C., Lin, B., Hong, A. W., Chun, J. V., and Guan, K. L. (2016). Characterization of hippo pathway components by gene inactivation. Mol. Cell 64, 993-1008.
[17] Tapon, N., Harvey, K. F., Bell, D. W., Wahrer, D. C. R., Schiripo, T. A., Haber, D. A., and Hariharan, I. K. (2002). Salvador promotes both cell cycle exit and apoptosis in drosophila and is mutated in human cancer cell lines. Cell 110, 467-478.
[18] Yin, F., Yu, J., Zheng, Y., Chen, Q., Zhang, N., and Pan, D. (2013). Spatial organization of hippo signaling at the plasma membrane mediated by the tumor suppressor merlin/NF2. Cell 154, 1342-1355.
[19] Yu, J., Zheng, Y., Dong, J., Klusza, S., Deng, W. M., and Pan, D. (2010). Kibra functions as a tumor suppressor protein that regulates hippo signaling in conjunction with merlin and expanded. Dev. Cell 18, 288-299.
[20] Zhang, N., Bai, H., David, K. K., Dong, J., Zheng, Y., Cai, J., Giovannini, M., Liu, P., Anders, R. A., and Pan, D. (2010). The Merlin/NF2 tumor suppressor functions through the YAP oncoprotein to regulate tissue homeostasis in mammals. Dev. Cell 19, 27-38.
[21] Dong, J., Feldmann, G., Huang, J., Wu, S., Zhang, N., Comerford, S. A., Gayyed, M. F., Anders, R. A., Maitra, A., and Pan, D. (2007). Elucidation of a universal size-control mechanism in Drosophila and mammals. Cell 130, 1120-1133.
[22] Liu, C. Y., Zha, Z. Y., Zhou, X., Zhang, H., Huang, W., Zhao, D., Li, T., Chan, S. W., Lim, C. J., Hong, W., et al. (2010). The hippo tumor pathway promotes TAZ degradation by phosphorylating a Phosphodegron and recruiting the SCF b-TrCP E3 ligase. J. Biol. Chem. 285, 37159-37169.
[23] Li, W., You, L., Cooper, J., Schiavon, G., Pepe-Caprio, A., Zhou, L., Ishii, R., Giovannini, M., Hanemann, C. O., Long, S. B., et al. (2010). Merlin/NF2 suppresses tumorigenesis by inhibiting the E3 ubiquitin ligase CRL4 (DCAF1) in the nucleus. Cell 140, 477-490.
[24] Li, W., Cooper, J., Zhou, L., Yang, C., Erdjument-Bromage, H., Zagzag, D., Snuderl, M., Ladanyi, M., Hanemann, C. O., Zhou, P., et al. (2014). Merlin/NF2 loss-driven tumorigenesis linked to CRL4(DCAF1)-mediated inhibition of the hippo pathway kinases Lats1 and 2 in the nucleus. Cancer Cell 26, 48-60.
[25] Benhamouche, S., Curto, M., Saotome, I., Gladden, A. B., Liu, C. H., Giovannini, M., and McClatchey, A. I. (2010). Nf2/Merlin controls progenitor homeostasis and tumorigenesis in the liver. Genes Dev. 24, 1718-1730.
[26] Croy, B. A., and Chapeau, C. (1990). Evaluation of the pregnancy immunotrophism hypothesis by assessment of the reproductive performance of young adult mice of genotype scid/scid.bg/bg. J. Reprod. Fertil. 88, 231-239.
[27] Lallemand, D., Manent, J., Couvelard, A., Watilliaux, A., Siena, M., Chareyre, F., Lampin, A., Niwa-Kawakita, M., Kalamarides, M., and Giovannini, M. (2009). Merlin regulates transmembrane receptor accumulation and signaling at the plasma membrane in primary mouse Schwann cells and in human schwannomas. Oncogene 28, 854-865.
[28] Gui, D. Y., Sullivan, L. B., Luengo, A., Hosios, A. M., Bush, L. N., Gitego, N., Davidson, S. M., Freinkman, E., Thomas, C. J., and Vander Heiden, M. G. (2016). Environment dictates dependence on mitochondrial complex I for NAD+ and aspartate production and determines cancer cell sensitivity to metformin. Cell Metab. 24, 716-727.
[29] Mackay, G. M., Zheng, L., van den Broek, N. J. F., and Gottlieb, E. (2015). Analysis of cell metabolism using LC-MS and isotope tracers. Methods Enzymol. 561, 171-196.
[30] Pierzyńska-Mach, A., Janowski, P. A., and Dobrucki, J. W. (2014). Evaluation of acridine orange, LysoTracker Red, and quinacrine as fluorescent probes for long-term tracking of acidic vesicles. Cytometry A 85, 729-737.
[31] Robinson, K. M., Janes, M. S., Pehar, M., Monette, J. S., Ross, M. F., Hagen, T. M., Murphy, M. P., and Beckman, J. S. (2006). Selective fluorescent imaging of superoxide in vivo using ethidium-based probes. Proc. Natl. Acad. Sci. USA 103, 15038-15043.
[32] Spadafora, D., Kozhukhar, N., Chouljenko, V. N., Kousoulas, K. G., and Alexeyev, M. F. (2016). Methods for efficient elimination of mitochondrial DNA from cultured cells. PLoS One 11, e0154684.
[33] Park, Y., Reyna-Neyra, A., Philippe, L., and Thoreen, C. C. (2017). mTORC1 balances cellular amino acid supply with demand for protein synthesis through post-transcriptional control of ATF4. Cell Rep. 19, 1083-1090.
[34] Dalgliesh, G. L., Furge, K., Greenman, C., Chen, L., Bignell, G., Butler, A., Davies, H., Edkins, S., Hardy, C., Latimer, C., et al. (2010). Systematic sequencing of renal carcinoma reveals inactivation of histone modifying genes. Nature 463, 360-363.
[35] Rossignol, R., Gilkerson, R., Aggeler, R., Yamagata, K., Remington, S. J., and Capaldi, R. A. (2004). Energy substrate modulates mitochondrial structure and oxidative capacity in cancer cells. Cancer Res. 64, 985-993.
[36] Barthel, A., Okino, S. T., Liao, J., Nakatani, K., Li, J., Whitlock, J. P., and Roth, R. A. (1999). Regulation of GLUT1 gene transcription by the serine/threonine kinase Aktl. J. Biol. Chem. 274, 20281-20286.
[37] Bentley, J., Itchayanan, D., Barnes, K., McIntosh, E., Tang, X., Downes, C. P., Holman, G. D., Whetton, A. D., Owen-Lynch, P. J., and Baldwin, S. A. (2003). Interleukin-3-mediated cell survival signals include phosphatidylinositol 3-kinase-dependent translocation of the glucose transporter GLUT1 to the cell surface. J. Biol. Chem. 278, 39337-39348.
[38] Deprez, J., Vertommen, D., Alessi, D. R., Hue, L., and Rider, M. H. (1997). Phosphorylation and activation of heart 6-phosphofructo-2-kinase by protein kinase B and other protein kinases of the insulin signaling cascades. J. Biol. Chem. 272, 17269-17275.
[39] Gottlob, K., Majewski, N., Kennedy, S., Kandel, E., Robey, R. B., and Hay, N. (2001). Inhibition of early apoptotic events by Akt/PKB is dependent on the first committed step of glycolysis and mitochondrial hexokinase. Genes Dev. 15, 1406-1418.
[40] Majewski, N., Nogueira, V., Bhaskar, P., Coy, P. E., Skeen, J. E., Gottlob, K., Chandel, N. S., Thompson, C. B., Robey, R. B., and Hay, N. (2004). Hexokinase-mitochondria interaction mediated by Akt is required to inhibit apoptosis in the presence or absence of Bax and Bak. Mol. Cell 16, 819-830.
[41] Rathmell, J. C., Fox, C. J., Plas, D. R., Hammerman, P. S., Cinalli, R. M., and Thompson, C. B. (2003). Akt-directed glucose metabolism can prevent Bax conformation change and promote growth factor-independent survival. Mol. Cell. Biol. 23,7315-7328.
[42] Wieman, H. L., Wofford, J. A., and Rathmell, J. C. (2007). Cytokine stimulation promotes glucose uptake via Phosphatidylinositol-3 kinase/Akt regulation of Glutl activity and trafficking. MBoC 18, 1437-1446.
[43] Coles, L. C., and Shaw, P. E. (2002). PAK1 primes MEK1 for phosphorylation by Raf-1 kinase during cross-cascade activation of the ERK pathway. Oncogene 21, 2236-2244.
[44] Eblen, S. T., Slack, J. K., Weber, M. J., and Catling, A. D. (2002). Rac-PAK signaling stimulates extracellular signal-regulated kinase (ERK) activation by regulating formation of MEK1-ERK complexes. Mol. Cell. Biol. 22, 6023-6033.
[45] King, A. J., Sun, H., Diaz, B., Barnard, D., Miao, W., Bagrodia, S., and Marshall, M. S. (1998). The protein kinase Pak3 positively regulates Raf-1 activity through phosphorylation of serine 338. Nature 396, 180-183.
[46] Slack-Davis, J. K., Eblen, S. T., Zecevic, M., Boerner, S. A., Tarcsafalvi, A., Diaz, H. B., Marshall, M. S., Weber, M. J., Parsons, J. T., and Catling, A. D. (2003). PAK1 phosphorylation of MEK1 regulates fibronectin-stimulated MAPK activation. J. Cell Biol. 162, 281-291.
[47] Cook, S. J., and Mccormick, F. (1993). Inhibition by cAMP of Ras-dependent activation of Raf. Science 262, 1069-1072.
[48] Dumaz, N., Light, Y., and Marais, R. (2002). Cyclic AMP blocks cell growth through Raf-1-dependent and Raf-1-independent mechanisms. Mol. Cell. Biol. 22, 3717-3728.
[49] Dumaz, N., Hayward, R., Martin, J., Ogilvie, L., Hedley, D., Curtin, J. A., Bastian, B. C., Springer, C., and Marais, R. (2006). In melanoma, RAS mutations are accompanied by switching signaling from BRAF to CRAF and disrupted cyclic AMP signaling. Cancer Res. 66, 9483-9491.
[50] Kim, J., Kwon, J., Kim, M., Do, J., Lee, D., and Han, H. (2016). A cardiac mitochondrial cAMP signaling pathway regulates calcium accumulation, permeability transition and cell death. Polym. J. 48, 829-834.
[51] Zippin, J. H., Farrell, J., Huron, D., Kamenetsky, M., Hess, K. C., Fischman, D. A., Levin, L. R., and Buck, J. (2004). Bicarbonate-responsive “soluble” adenylyl cyclase defines a nuclear cAMP microdomain. J. Cell Biol. 164, 527-534.
[52] Casey, J. R., Grinstein, S., and Orlowski, J. (2010). Sensors and regulators of intracellular pH. Nat. Rev. Mol. Cell Biol. 11, 50-61.
[53] Gunter, T. E., Gunter, K. K., Sheu, S. S., and Gavin, C. E. (1994). Mitochondrial calcium transport: physiological and pathological relevance. Am. J. Physiol. 267, C313-C339.
[54] McCormack, J. G., Halestrap, A. P., and Denton, R. M. (1990). Role of calcium ions in regulation of mammalian intramitochondrial metabolism. Physiol. Rev. 70, 391-425.
[55] Perera, R. M., Stoykova, S., Nicolay, B. N., Ross, K. N., Fitamant, J., Boukhali, M., Lengrand, J., Deshpande, V., Selig, M. K., Ferrone, C. R., et al. (2015). Transcriptional control of autophagy-lysosome function drives pancreatic cancer metabolism. Nature 524, 361-365.
[56] Zhang, X., Yu, L., and Xu, H. (2016). Lysosome calcium in ROS regulation of autophagy. Autophagy 12, 1954-1955.
[57] Aits, S., and Jäättelä, M. (2013). Lysosomal cell death at a glance. J. Cell Sci. 126, 1905-1912.
[58] Martinez-Lopez, N., Athonvarangkul, D., Mishall, P., Sahu, S., and Singh, R. (2013). Autophagy proteins regulate ERK phosphorylation. Nat. Commun. 4, 2799.
[59] Settembre, C., Di Malta, C., Polito, V. A., Garcia Arencibia, M., Vetrini, F., Erdin, S. U. S., Erdin, S. U. S., Huynh, T., Medina, D., Colella, P., et al. (2011). TFEB links autophagy to lysosomal biogenesis. Science 332, 1429-1433.
[60] Chen, F., Zhang, Y., Senbabaoğlu, Y., Ciriello, G., Yang, L., Reznik, E., Shuch, B., Micevic, G., De Velasco, G., Shinbrot, E., et al. (2016a). Multilevel genomics-based taxonomy of renal cell carcinoma. Cell Rep. 14, 2476-2489.
[61] Cancer Genome Atlas Research Network, Linehan, W. M., Spellman, P. T., Ricketts, C. J., Creighton, C. J., Fei, S. S., Davis, C., Wheeler, D. A., Murray, B. A., Schmidt, L., et al. (2016). Comprehensive molecular characterization of papillary renal-cell carcinoma. N. Engl. J. Med. 374, 135-145.

What is claimed is:

1. A method of treating or preventing cancer in a subject in need thereof, the method comprising administering a therapeutically effective amount of one or more inhibitors of the YAP/TAZ pathway to the subject.

2. The method of claim 1, wherein the cancer is selected from the group consisting of blood cancer, leukemia, lymphoma, skin cancer, melanoma, breast cancer, ovarian cancer, uterine cancer, prostate cancer, testicular cancer, colorectal cancer, stomach cancer, intestinal cancer, bladder cancer, lung cancer, non-small cell lung cancer, pancreatic cancer, renal cell carcinoma, kidney cancer, liver cancer, hepatocarcinoma, brain cancer, head and neck cancer, retinal cancer, glioma, lipoma, throat cancer, thyroid cancer, neuroblastoma, endometrial cancer, myelomas, mesothelioma, and esophageal cancer.

3. A method of treating or preventing noncancerous tumors or lesions in a subject in need thereof, the method comprising administering a therapeutically effective amount of one or more inhibitors of the YAP/TAZ pathway to the subject.

4. The method of claim 3, wherein the noncancerous tumors or lesions are associated with neurofibromatosis type 2 (NF2).

5. The method of claim 4, wherein the noncancerous tumors or lesions are selected from vestibular schwannomas, meningiomas, ependymomas, or a combination thereof

6. A method of inhibiting or preventing glycolysis in cancer cells in a subject in need thereof, the method comprising administering a therapeutically effective amount of one or more inhibitors of the YAP/TAZ pathway to the subject.

7. A method of promoting or inducing mitochondrial respiration in cancer cells in a subject in need thereof, the method comprising administering a therapeutically effective amount of one or more inhibitors of the YAP/TAZ pathway to the subject.

8. A method of promoting or inducing mitochondrial respiration in cancer cells in a subject in need thereof, the method comprising administering a therapeutically effective amount of one or more inhibitors of the YAP/TAZ pathway to the subject.

9. A method of promoting or inducing oxidative stress in cancer cells in a subject in need thereof, the method comprising administering a therapeutically effective amount of one or more inhibitors of the YAP/TAZ pathway to the subject.

10. A method of promoting or inducing lysosome-mediated activation of MAPK signaling in cancer cells in a subject in need thereof, the method comprising administering a therapeutically effective amount of one or more inhibitors of the YAP/TAZ pathway to the subject.

11. The method of any one of claims 6-10, wherein the cancer cells are selected from the group consisting of skin cancer cells, breast cancer cells, ovarian cancer cells, uterine cancer cells, prostate cancer cells, testicular cancer cells, colorectal cancer cells, stomach cancer cells, intestinal cancer cells, bladder cancer cells, lung cancer cells, non-small cell lung cancer cells, pancreatic cancer cells, kidney cancer cells, liver cancer cells, brain cancer cells, head and neck cancer cells, retinal cancer cells, throat cancer cells, thyroid cancer cells, endometrial cancer cells, and esophageal cancer cells.

12. The method of any one of claims 1-11, wherein the one or more inhibitors comprise verteporfin, (R)-PFI 2 hydrochloride, CA3, dasatinib, statins, pazopanib, β-adrenergic receptor agonists, dobutamine, latrunculin A, latrunculin B, cytochalasin D, actin inhibitors, drugs that act on the cytoskeleton, blebbistatitin, botulinum toxin C3, RHO kinase-targeting drugs, or a combination thereof

13. The method of any one of claims 1-12, wherein the administration of the one or more inhibitors of the YAP/TAZ pathway is preceded by a step of identifying the subject in need thereof.

14. The method of any one of claims 1-13, further comprising administering one or more inhibitors of mitogen-activated protein kinase (MAPK) signaling to the subject.

15. The method of claim 14, wherein the one or more inhibitors of MAPK signaling comprises one or more inhibitors of rapidly accelerated fibrosarcoma (RAF)—mitogen-activated extracellular signal-regulated kinase (MEK)—extracellular signal-regulated kinases (ERK) pathway (RAF-MEK-ERK pathway).

16. The method of claim 15, wherein the one or more inhibitors of MAPK signaling comprises trametinib, cobimetinib, binimetinib, refametinib, selumetinib, or a combination thereof.


SEQUENCE LISTING (6 sequences)

SEQ ID NO: 1 - DNA, 22 bp, Artificial Sequence
  Description of Artificial Sequence Synthetic oligonucleotide
gtgccaccaagctagataaaga

SEQ ID NO: 2 - DNA, 22 bp, Artificial Sequence
  Description of Artificial Sequence Synthetic oligonucleotide
ggcatcttggtccaggaaatgt

SEQ ID NO: 3 - DNA, 25 bp, Artificial Sequence
  Description of Artificial Sequence Synthetic primer
tgctgtctccatgtttgatgtatct

SEQ ID NO: 4 - DNA, 22 bp, Artificial Sequence
  Description of Artificial Sequence Synthetic primer
tctctgctccccacctctaagt

SEQ ID NO: 5 - DNA, 20 bp, Artificial Sequence
  Description of Artificial Sequence Synthetic primer
cacccaagaacagggtttgt

SEQ ID NO: 6 - DNA, 20 bp, Artificial Sequence
  Description of Artificial Sequence Synthetic primer
tggccatgggtatgttgtta